(19) United States
(12) Patent Application Publication
(10) Pub. No.: US 2024/0166624 A1
(43) Pub. Date: May 23, 2024

(54) PPARG INVERSE AGONISTS AND USES THEREOF

(71) Applicant: Flare Therapeutics Inc., Cambridge, MA (US)

(72) Inventors: James E. Audia, Chicago, IL (US); Jacob I. Stuckey, Framingham, MA (US); Byron DeLaBarre, Arlington, MA (US); Jonathan E. Wilson, Arlington, MA (US)

(21) Appl. No.: 18/279,292

(22) PCT Filed: Mar. 1, 2022
(86) PCT No.: PCT/US2022/018283

§ 371 Date: Aug. 29, 2023

Related U.S. Application Data
(60) Provisional application No. 63/155,410, filed on Mar. 2, 2021.

(51) Int. Cl. C07D 401/04; C07D 215/233; C07D 215/38; C07D 215/48; C07D 405/04; C07D 487/10; C07D 491/107

(52) U.S. Cl. CPC C07D 401/04; C07D 215/233; C07D 215/38; C07D 215/48; C07D 405/04; C07D 487/10; C07D 491/107

(57) ABSTRACT

Provided are compounds of Formula (I): (I); and pharmaceutically acceptable salts and compositions thereof, which are useful for treating a variety of conditions associated with PPARG.

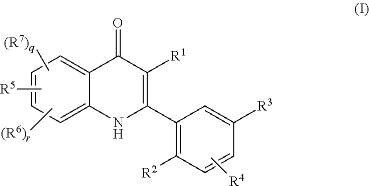

### RELATED APPLICATIONS

This application claims the benefit of priority to U.S. Provisional Application No. 63/155,410, filed Mar. 2, 2021, the entire contents of which are incorporated herein by reference.

### BACKGROUND

PPARgamma (PPARG) is a type II ligand-dependent nuclear hormone receptor (belonging to the PPAR nuclear receptor subfamily) that functions as an obligate heterodimer with retinoid X receptors (RXRs). PPARG is predominantly expressed in adipose tissue, colon, macrophages and the luminal layers of the urothelium. PPARG is known as a master regulator of adipogenesis, functioning to regulate adipocyte differentiation, fatty acid storage and glucose metabolism. PPARG has also been shown to play an important role in the metabolism and inflammation of macrophages, where it is induced by IL4 and controls glutamine metabolism. In the normal urothelium, PPARG is critical for its homeostasis and regeneration.

The role for PPARG in cancer was originally inferred from genomic studies that identified a PAX8-PPARG chromosomal rearrangement in follicular thyroid carcinomas. More recently, PPARG has been found to be over-expressed and genetically altered in the luminal subtype of urothelial cancer. This is consistent with reports that long-term use of PPARG agonists is associated with an increased incidence of urothelial cancer. Most urothelial cancers are urothelial carcinoma, which are classified as either non-muscle-invasive urothelial cancer (NMIUC, 70%), muscle-invasive urothelial cancer (MIUC, 25%) or metastatic urothelial cancer (MUC, 5%). MIUC is usually diagnosed de novo but may arise from the 10 to 20% of NMIUC cases that eventually progress. MIUC is a heterogeneous and aggressive disease, associated with a five-year survival rate of 60% for patients with localized disease and less than 10% for patients with distant metastases. Molecular understanding of NMIUC and MIUC has improved significantly, including the association between molecular subtypes and urothelial differentiation. Several molecular classes of MIUC have been proposed, whereby an activated PPARG signature features prominently in the luminal subtypes. First-line treatment is chemotherapy with several options in chemo-ineligible or second line, but treatment options are limited with poor overall survival rates.

The need exists to develop effective PPARG modulators for treating cancers such as NMIUC, MIUC, and MUC, and related conditions.

### SUMMARY

Provided herein are compounds having the Formula I:

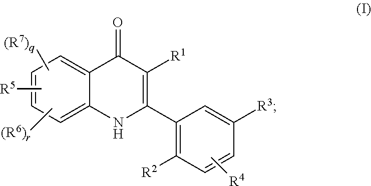

and pharmaceutically acceptable salts and compositions thereof, wherein R¹, R², R³, R⁴, R⁵, R⁶, R⁷, q and r are as described herein. In one aspect, the disclosed compounds of Formula I and pharmaceutically acceptable salts thereof modulate PPARG (e.g., as agonists such as inverse agonists, and are useful in a variety of therapeutic applications such as, for example, in treating cancer. As such, their uses for treating diseases responsive to the inhibition of PPARG are included.

Pharmaceutical compositions comprising the compounds and pharmaceutically acceptable salts of the disclosed compounds of Formula I, as well as methods for their preparation are also included.

### DETAILED DESCRIPTION

### 1. General Description of Compounds

In a first embodiment, provided herein is a compound of Formula I:

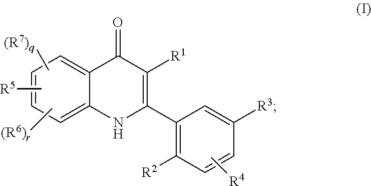

or a pharmaceutically acceptable salt thereof, wherein:
R¹ is hydrogen, halo, (C₁-C₄)alkyl, or hydroxyl;
   R² is halo;
   R³ is cyano or nitro;
   R⁴ is hydrogen, halo, (C₁-C₄)alkyl, (C₁-C₄)alkoxy, or hydroxyl;
   R⁵ is halo, halo(C₁-C₄)alkyl, or cyano;
   R⁶ is halo, halo(C₁-C₄)alkyl, (C₁-C₄)alkyl, or cyano;
   R⁷ is halo, (C₁-C₄)alkyl, (C₁-C₄)alkoxy, halo(C₁-C₄)alkyl, halo(C₁-C₄)alkoxy, —(C₁-C₄)alkylOR^{a}, —(C₁-C₄)alkylC(O)R^{a}, —(C₁-C₄)alkylC(O)OR^{a}, —C(O)NR^{a}R^{b}, —(C₁-C₄)alkylC(O)NR^{a}R^{b}, —C(O)R^{a}, —C(O)OR^{a}, —NR^{a}R^{b}, —(C₁-C₄)alkylNR^{a}R^{b}, —C(O)NR^{a}SO₃H, —NR^{a}C(O)R^{b}, —NR^{a}C(O)OR^{b}, —NR^{a}C(S)OR^{b}, —NR^{c}C(O)N^{a}R^{b}, —NR^{c}C(S)NR^{a}R^{b}, —NR^{c}S(O)₂NR^{a}R^{b}, —C(S)R^{a}, —S(O)₂R^{a}, —S(O)R^{a}, —C(S)OR^{a}, —C(S)NR^{a}R^{b}, —NR^{a}C(S)R^{b}, —SR^{a}, phenyl, 4- to 6-membered heterocyclyl, and 5- to 7-membered heteroaryl, wherein each of said phenyl, 4- to 6-membered heterocyclyl, and 5- to 7-membered heteroaryl are optionally and independently substituted with 1 to 3 groups selected from R⁸;
   R⁸ is selected from halo, (C₁-C₄)alkyl, halo(C₁-C₄)alkyl, (C₁-C₄)alkoxy, halo(C₁-C₄)alkoxy, nitro, oxo, cyano, —(C₁-C₄)alkylOR^{d}, —(C₁-C₄)alkylC(O)R^{d}, —(C₁-C₄)alkylC(O)OR^{d}, —C(O)NR^{d}R^{e}, —(C₁-C₄)alkylC(O)NR^{d}R^{e}, —C(O)R^{d}, —C(O)OR^{d}, —NR^{d}R^{e}, —(C₁-C₄)alkylNR^{d}R^{e}, —C(O)NR^{d}SO₃H, —NR^{d}C(O)R^{e}, —NR^{d}C(O)OR^{e}, —NR^{d}C(S)OR^{e}, —NR^{f}C(O)N^{d}R^{e}, —NR^{f}C(S)NR^{d}R^{e}, —NR^{f}S(O)₂NR^{d}R^{e}, —C(S)R^{d}, —S(O)₂R^{d}, —S(O)R^{d}, —C(S)OR^{d}, —C(S)NR^{d}R^{e}, —NR^{d}C(S)R^{e}, and —SR^{d};
   R^{a}, R^{b}, R^{c}, R^{d}, R^{e}, and R^{f} are each independently hydrogen or (C₁-C₄)alkyl; and
   q and r are each independently 0 or 1.

### 2. Definitions

When used in connection to describe a chemical group that may have multiple points of attachment, a hyphen (-) designates the point of attachment of that group to the variable to which it is defined. For example, —NR^{b}C(O)OR^{c} and —NR^{b}C(S)OR^{c} mean that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical.

“Alkoxy” means an alkyl radical attached through an oxygen linking atom, represented by —O-alkyl. For example, “(C₁-C₄)alkoxy” includes methoxy, ethoxy, proproxy, and butoxy.

The term “haloalkyl” includes mono, poly, and perhaloalkyl groups where the halogens are independently selected from fluorine, chlorine, bromine, and iodine.

“Haloalkoxy” is a haloalkyl group which is attached to another moiety via an oxygen atom such as, e.g., —OCHF₂ or —OCF₃.

The term oxo means the group ═O.

The term “5- to 7-membered heteroaryl” used alone or as part of a larger moiety refers to a 5- to 7-membered aromatic radical containing 1-4 heteroatoms selected from N, O, and S. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Optional substituents on a heteroaryl group may be present on any substitutable position and, include, e.g., the position at which the heteroaryl is attached.

The term “4- to 6-membered heterocyclyl” means a 4- to 6-membered saturated or partially unsaturated heterocyclic ring containing 1 to 4 heteroatoms independently selected from N, O, and S. A heterocyclyl ring can be attached to its pendant group at any heteroatom or carbon atom that results in a stable structure. Examples of monocyclic saturated or partially unsaturated heterocyclic radicals include, without limitation, tetrahydrofuranyl, tetrahydrothienyl, tetrahydropyranyl, pyrrolidinyl, pyrrolidonyl, piperidinyl, oxazolidinyl, piperazinyl, dioxanyl, oxetanyl, dioxolanyl, morpholinyl, dihydrofuranyl, dihydropyranyl, dihydropyridinyl, tetrahydropyridinyl, dihydropyrimidinyl, and tetrahydropyrimidinyl. Optional substituents on a heterocyclyl group may be present on any substitutable position and, include, e.g., the position at which the heterocyclyl is attached.

The disclosed compounds may exist in one or more tautomeric forms, such as those below, and are included herein.

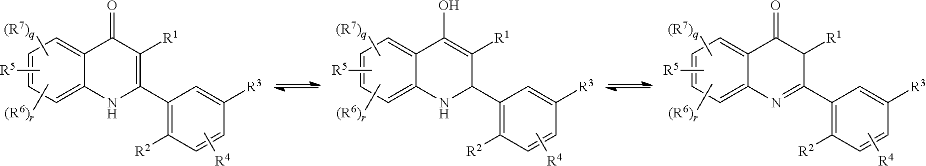

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g., salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Compounds

In a second embodiment, the compound of Formula I is of the Formula II:

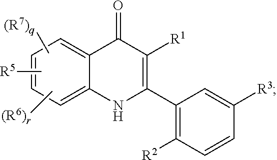

or a pharmaceutically acceptable salt thereof, wherein the variables are as described above for Formula I.

In a third embodiment, R² in the compound of Formula I or II, or a pharmaceutically acceptable salt thereof is chloro, wherein the remaining variables are as described above for Formula I.

In a fourth embodiment, R³ in the compound of Formula I or II, or a pharmaceutically acceptable salt thereof is cyano, wherein the remaining variables are as described above for Formula I or the third embodiment.

In a fifth embodiment, R¹ in the compound of Formula I or II, or a pharmaceutically acceptable salt thereof is hydrogen, fluoro, hydroxyl, or methyl, wherein the remaining variables are as described above for Formula I or the third or fourth embodiment. Alternatively, as part of a fifth embodiment, R¹ in the compound of Formula I or II, or a pharmaceutically acceptable salt thereof is hydrogen, fluoro, hydroxyl, or methyl, wherein the remaining variables are as described above for Formula I or the third or fourth embodiment. In another alternative, as part of a fifth embodiment, R¹ in the compound of Formula I or II, or a pharmaceutically acceptable salt thereof is hydrogen, wherein the remaining variables are as described above for Formula I or the third or fourth embodiment.

In a sixth embodiment, R⁵ in the compound of Formula I or II, or a pharmaceutically acceptable salt thereof is halo or cyano, wherein the remaining variables are as described above for Formula I or the third, fourth, or fifth embodiment. Alternatively, as part of a sixth embodiment, R⁵ in the compound of Formula I or II, or a pharmaceutically acceptable salt thereof is halo, wherein the remaining variables are as described above for Formula I or the third, fourth, or fifth embodiment. In another alternative, as part of a sixth embodiment, R⁵ in the compound of Formula I or II, or a pharmaceutically acceptable salt thereof is chloro or fluoro, wherein the remaining variables are as described above for Formula I or the third, fourth, or fifth embodiment. In another alternative, as part of a sixth embodiment, R⁵ in the compound of Formula I or II, or a pharmaceutically acceptable salt thereof is fluoro, wherein the remaining variables are as described above for Formula I or the third, fourth, or fifth embodiment.

In a seventh embodiment, q in the compound of Formula I or II, or a pharmaceutically acceptable salt thereof is 1, wherein the remaining variables are as described above for Formula I or the third, fourth, fifth, or sixth embodiment.

In an eighth embodiment, r in the compound of Formula I or II, or a pharmaceutically acceptable salt thereof is 1, wherein the remaining variables are as described above for Formula I or the third, fourth, fifth, sixth, or seventh embodiment. Alternatively, as part of an eighth embodiment, r in the compound of Formula I or II, or a pharmaceutically acceptable salt thereof is 0, wherein the remaining variables are as described above for Formula I or the third, fourth, fifth, sixth, or seventh embodiment.

In a ninth embodiment, R⁶ in the compound of Formula I or II, or a pharmaceutically acceptable salt thereof is halo, wherein the remaining variables are as described above for Formula I or the third, fourth, fifth, sixth, or seventh embodiment. Alternatively, as part of a ninth embodiment, R⁶ in the compound of Formula I or II, or a pharmaceutically acceptable salt thereof is fluoro, wherein the remaining variables are as described above for Formula I or the third, fourth, fifth, sixth, or seventh embodiment.

In a tenth embodiment, R⁷ in the compound of Formula I or II, or a pharmaceutically acceptable salt thereof is halo, halo(C₁-C₄)alkyl, (C₁-C₄)alkyl, (C₁-C₄)alkoxy, —(C₁-C₄)alkylOR^{a}, —C(O)NR^{a}R^{b}, phenyl, 4- to 6-membered heterocyclyl, and 5- to 7-membered heteroaryl, wherein each of said phenyl, 4- to 6-membered heterocyclyl, and 5- to 7-membered heteroaryl are optionally and independently substituted with 1 to 3 groups selected from R⁸, wherein the remaining variables are as described above for Formula I or the third, fourth, fifth, sixth, seventh, eighth, or ninth embodiment. Alternatively, as part of a tenth embodiment, R⁷ in the compound of Formula I or II, or a pharmaceutically acceptable salt thereof is halo, halo(C₁-C₄)alkyl, (C₁-C₄)alkyl, (C₁-C₄)alkoxy, —(C₁-C₄)alkylOR^{a}, —C(O)NR^{a}R^{b}, phenyl, pyridinyl, pyrazolyl, and oxetanyl, wherein each of said phenyl, pyridinyl, pyrazolyl, and oxetanyl are optionally and independently substituted with 1 to 3 groups selected from R⁸, wherein the remaining variables are as described above for Formula I or the third, fourth, fifth, sixth, seventh, eighth, or ninth embodiment. In another alternative, as part of a tenth embodiment, R⁷ in the compound of Formula I or II, or a pharmaceutically acceptable salt thereof is halo, halo(C₁-C₄)alkyl, (C₁-C₄)alkyl, (C₁-C₄)alkoxy, —(C₁-C₄)alkylOR^{a}, —(C₁-C₄)alkylC(O)NR^{a}R^{b}, —(C₁-C₄)alkylC(O)OR^{a}, —C(O)NR^{a}R^{b}, phenyl, 4- to 6-membered heterocyclyl, and 5- to 7-membered heteroaryl, wherein each of said phenyl, 4- to 6-membered heterocyclyl, and 5- to 7-membered heteroaryl are optionally and independently substituted with 1 to 3 groups selected from R⁸, wherein the remaining variables are as described above for Formula I or the third, fourth, fifth, sixth, seventh, eighth, or ninth embodiment. In another alternative, as part of a tenth embodiment, R⁷ in the compound of Formula I or II, or a pharmaceutically acceptable salt thereof is halo, halo(C₁-C₄)alkyl, (C₁-C₄)alkyl, (C₁-C₄)alkoxy, —(C₁-C₄)alkylC(O)NR^{a}R^{b}, —(C₁-C₄)alkylOR^{a}, —(C₁-C₄)alkylC(O)OR^{a}, —C(O)NR^{a}R^{b}, azetidinyl, phenyl, pyridinyl, piperazinyl, piperidinyl, pyridinyl, pyrazolyl, tetrahydropyridinyl, pyrrolidinyl, pyrazinyl, dihydropyridazinyl, pyridazinyl, imadazolyl, dihydropyridinyl, dihydropyrimidinyl, pyrimidinyl, and oxetanyl, wherein each of said azetidinyl, phenyl, pyridinyl, piperazinyl, piperidinyl, pyridinyl, pyrazolyl, tetrahydropyridinyl, pyrrolidinyl, pyrazinyl, dihydropyridazinyl, pyridazinyl, imadazolyl, dihydropyridinyl, dihydropyrimidinyl, pyrimidinyl, and oxetanyl are optionally and independently substituted with 1 to 3 groups selected from R⁸, wherein the remaining variables are as described above for Formula I or the third, fourth, fifth, sixth, seventh, eighth, or ninth embodiment.

In an eleventh embodiment, R⁸ in the compound of Formula I or II, or a pharmaceutically acceptable salt thereof is selected from halo, (C₁-C₄)alkyl, halo(C₁-C₄)alkyl, (C₁-C₄)alkoxy, halo(C₁-C₄)alkoxy, oxo, and cyano, wherein the remaining variables are as described above for Formula I or the third, fourth, fifth, sixth, seventh, eighth, ninth, or tenth embodiment. Alternatively, as part of an eleventh embodiment, R⁸ in the compound of Formula I or II, or a pharmaceutically acceptable salt thereof is halo(C₁-C₄)alkyl, wherein the remaining variables are as described above for Formula I or the third, fourth, fifth, sixth, seventh, eighth, ninth, or tenth embodiment. In another alternative, as part of an eleventh embodiment, R⁸ in the compound of Formula I or II, or a pharmaceutically acceptable salt thereof is selected from halo, (C₁-C₄)alkyl, halo(C₁-C₄)alkyl, (C₁-C₄)alkoxy, —(C₁-C₄)alkylOR^{d}, —(C₁-C₄)alkylNR^{d}R^{e}, —(C₁-C₄)alkylC(O)OR^{d}, halo(C₁-C₄)alkoxy, —C(O)OR^{d}, —(C₁-C₄)alkylC(O)NR^{d}R^{e}, —C(O)NR^{d}R^{e}, oxo, cyano, —C(O)R^{d}, —NR^{d}R^{e}, and —S(O)₂R^{d}, wherein the remaining variables are as described above for Formula I or the third, fourth, fifth, sixth, seventh, eighth, ninth, or tenth embodiment.

Compounds having the Formula I and II are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of PPARG. In some aspects, the compounds, pharmaceutical acceptable salts, and pharmaceutical compositions described herein inhibit the activity PPARG. In some aspects, the compounds and pharmaceutical acceptable salts disclosed herein are agonists of PPARG. In some aspects, the compounds and pharmaceutical acceptable salts disclosed herein are agonists of PPARG. In some aspects, the compounds and pharmaceutical acceptable salts disclosed herein are inverse agonists of PPARG. In one aspect, “inverse-agonists” refer to agents that bind to the same receptor binding site as a agonist (e.g., the binding site of a nuclear receptor such as PPARG) and not only antagonizes the effects of an agonist but, moreover, exerts the opposite effect by suppressing spontaneous receptor signaling (when present).

In some aspects, the compounds and pharmaceutical acceptable salts disclosed herein overcome the activated state of PPARG function resulting from alteration in PPARG activity (mutation, amplification or overexpression) or from RXRA activating mutations. In some aspect, the compounds and pharmaceutical acceptable salts disclosed herein increase the repressive state (NCOR1 recruitment) to a higher degree than previously disclosed PPARG modulators such as prior inverse agonists. Such results even arise in the mutant context. See e.g., the table qualitatively assessing NCOR1 recruitment and repression of PPARG target genes in HT1197 in the Exemplification section.

In some aspects, the compounds and pharmaceutical compositions described herein are useful in treating a disorder associated with PPARG function. Thus, provided herein are methods of treating a disorder associated with PPARG function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof.

Also provided is the use of a compound described herein, or a pharmaceutically acceptable salt thereof, or a pharmaceutical composition comprising a disclosed compound or pharmaceutically acceptable salt thereof, for the manufacture of a medicament for treating a disorder associated with PPARG function. Also provided is a compound described herein, or a pharmaceutically acceptable salt thereof, or a pharmaceutical composition comprising a disclosed compound or pharmaceutically acceptable salt thereof, for use in treating a disorder associated with PPARG.

In one aspect, the disorder associated with PPARG is cancer. In some aspects, the cancer is associated with an up-regulated peroxisome proliferator-activated receptor (PPAR) signaling pathway. In some aspects, the up-regulated PPAR signaling pathway is associated with increased expression of one or more genes selected from Uroplakin 1A (UPK1A), Uroplakin IB (UPK1B), Uroplakin (UPK2), Keratin 20 (KRT20), GATA Binding Protein 3 (GAT A3), Nuclear Receptor Corepressor 1 (NCOR1), Nuclear Receptor Corepressor 2 (NCOR2), Fatty Acid Binding Protein 4 (FABP4), Forkhead Box A1 (FOXA1), CD36 Molecule (CD36), Acyl-CoA Oxidase 1 (ACOX1), 3-Hydroxy-3-Methylglutaryl-CoA Synthase 2 (HMGCS2), Acyl-CoA Synthetase Long-Chain Family Member 5 (ACSL5), Arachidonate 5-Lipoxygenase (ALOX5), Acyl-CoA Synthetase Long-Chain Family Member 1 (ACSL1), and Angiopoietin Like 4 (ANGPTL4).

In some aspects, the cancer treated by the compounds, pharmaceutically acceptable salt thereof, and pharmaceutical compositions described herein is selected from breast cancer, pancreatic cancer, ovarian cancer, prostate cancer, renal cancer, bladder cancer, testicular cancer, urothelial cancer (e.g., non-muscle-invasive urothelial cancer, muscle-invasive urothelial cancer, metastatic urothelial cancer), skin cancer, melanoma, colon cancer, kidney cancer, brain cancer and a hematopoietic cancer (e.g., lymphoma, multiple myeloma and leukemia). In one aspect, the cancer treated by the compounds, pharmaceutically acceptable salt thereof, and pharmaceutical compositions described herein is urothelial cancer such as non-muscle-invasive urothelial cancer, muscle-invasive urothelial cancer, and metastatic urothelial cancer.

Other uses besides cancer are contemplated and include e.g., metabolic diseases (e.g., osteoporosis, rachitis, arthrosis, obesity, type I and type II diabetes mellitus), lipid metabolism disorder, pancreatitis, glucose metabolism disorder, diabetic neuropathy, diabetic complications, hyperuricemia, osteoporosis, rachitis, arthrosis inflammatory diseases (e.g., inflammatory skin diseases such as psoriasis, atopic dermatitis, eczema, acne vulgaris, other dermatitides and pruritus), pulmonary disorders (e.g., asthma and chronic obstructive pulmonary disease), autoimmune disease, neurodegenerative disease (e.g., multiple sclerosis, Alzheimer's disease, and Parkinson's disease), cardiovascular diseases (e.g., selected from atherosclerosis, venous and arterial occlusive diseases), restenosis after invasive procedures, cardiomyopathy, myocardial fibrosis, congestive heart failure, angiogenesis and neovascularization in neoplastic diseases and renal diseases.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

### Chemical Synthesis

The representative examples that follow are intended to help illustrate the present disclosure, and are not intended to, nor should they be construed to, limit the scope of the invention.

General starting materials used were obtained from commercial sources or prepared in other examples, unless otherwise noted.

### Preparation of Compounds

The compounds claimed herein were prepared following the procedures outlined in the following schemes.

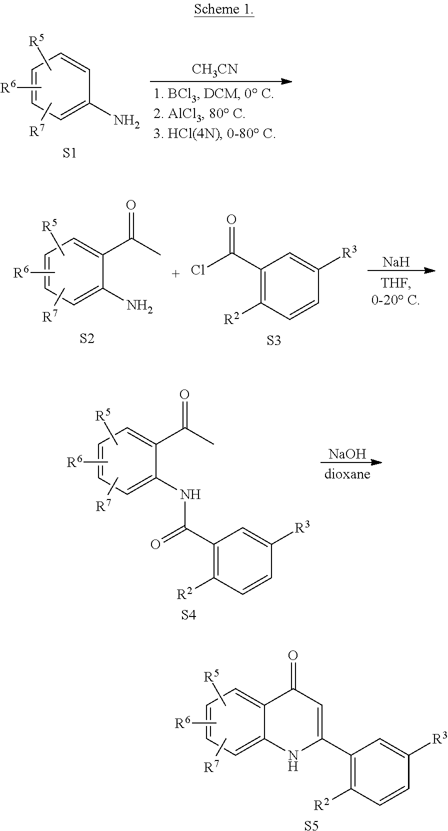

Quinolones like S5 may be prepared by the general synthetic methods shown in Scheme 1. Compounds of formula S2 may be prepared from the anilines S1 by treatment with acetonitrile, boron trichloride, aluminum trichloride and HCl in an organic solvent such as dichloromethane. Treatment of the acetylaniline S2 with a base such as sodium hydride in an organic solvent such as THF and treatment with an acyl chloride S3 yields intermediates of formula S4. Quinolones like S5 may then be prepared from S4 by treatment with base such as sodium hydroxide in an organic solvent such as dioxane at elevated temperature. Acyl chlorides S3 may be prepared from the corresponding acid by treatment with thionyl chloride or oxalyl chloride in an organic solvent such as dichloromethane.

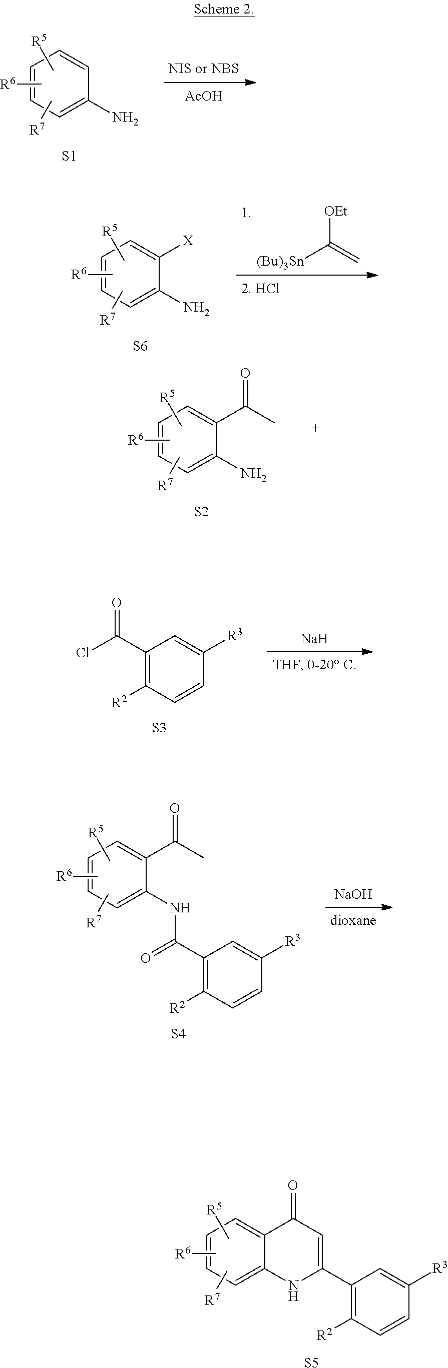

Quinolones like S5 may also be prepared by the general synthetic methods as shown in Scheme 2. 2-halo-anilines such as S6 may be prepared from aniline S1 upon treatment with NIS or NBS in an organic solvent such as acetic acid. Compounds of formula S2 can be prepared from the 2-halogen-aniline S6 by treatment with tributyl(1-ethoxyvinyl)stannane and a palladium catalyst such as Pd(PPh₃)₄ in an organic solvent such as toluene at an elevated temperature followed by treatment with aqueous acidic solution such as hydrochloric acid. Treatment of the acetylaniline S2 with a base such as sodium hydride in an organic solvent such as THF and treatment with an acyl chloride S3 yields intermediates of formula S4. Quinolones like S5 may then be prepared from S4 by treatment with base such as sodium hydroxide in an organic solvent such as dioxane at elevated temperature. Acyl chlorides S3 may be prepared from the corresponding acid by treatment with thionyl chloride or oxalyl chloride in an organic solvent such as dichloromethane.

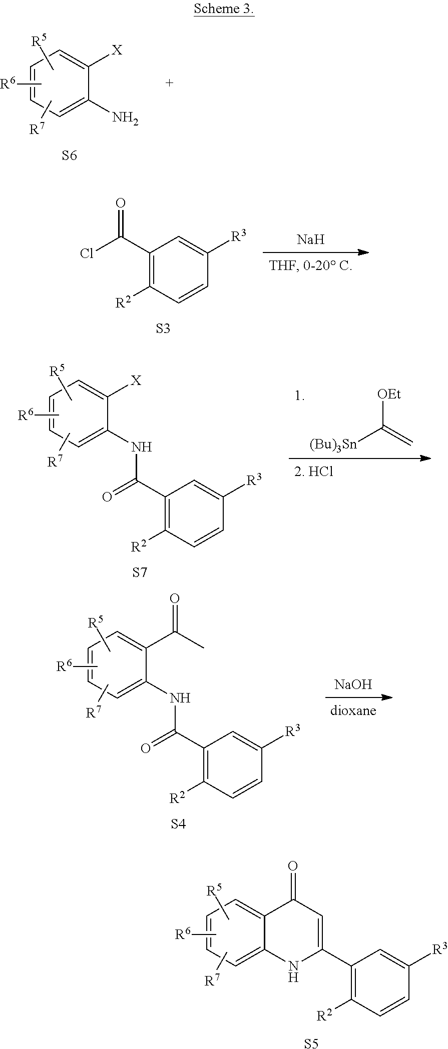

Quinolones like S5 may also be prepared by the general synthetic methods as shown in Scheme 3. Deprotonation of a 2-haloaniline S6 with a base such as sodium hydride NaH in an organic solvent, such as THF, and treatment with an acyl chloride S4 to yields the intermediate S7. S7 can be converted to S4 upon treatment with tributyl(1-ethoxyvinyl)stannane and a palladium catalyst, such as Pd(PPh₃)₄, in an organic solvent, such as toluene, followed by treatment with aqueous acid. Quinolones like S5 may then be prepared from S4 by treatment with base such as sodium hydroxide in an organic solvent such as dioxane at elevated temperature.

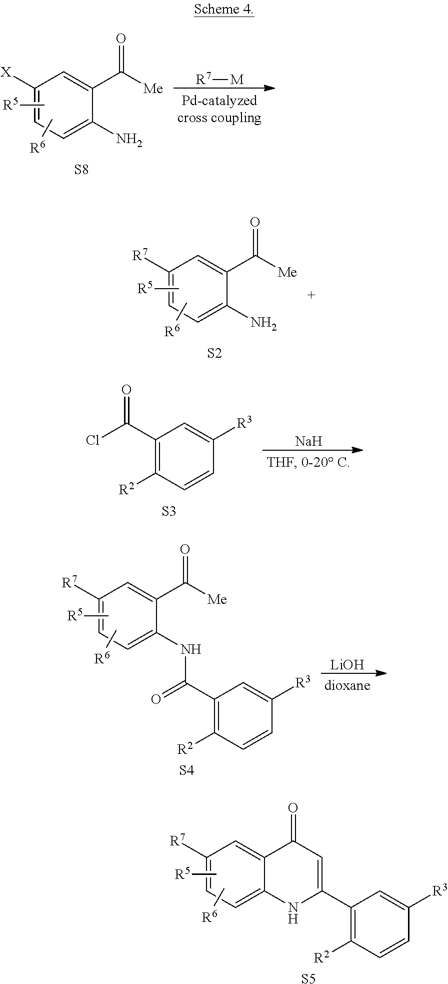

Quinolones like S5 may also be prepared by the general synthetic methods as shown in Scheme 4. Various palladium catalyzed cross couplings of S8 may be used to prepare substituted acetylaniline intermediates S2. Treatment of S2 with a base such as sodium hydride in an organic solvent such as THF and treatment with an acyl chloride S3 yields intermediates of formula S4. Quinolones like S5 may then be prepared from S4 by treatment with base such as sodium hydroxide in an organic solvent such as dioxane at elevated temperature.

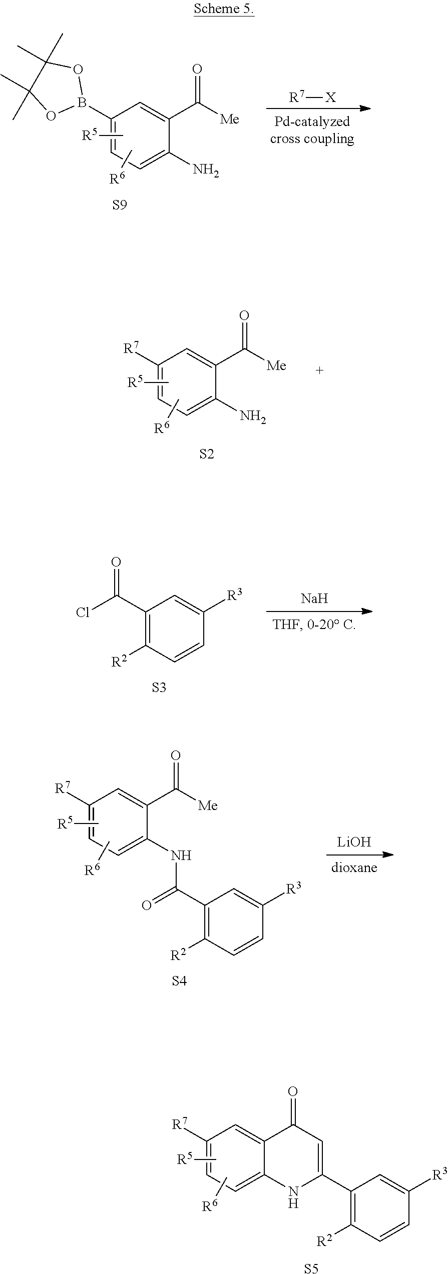

Quinolones like S5 may also be prepared by the general synthetic methods as shown in Scheme 5. Various palladium catalyzed cross couplings of S9 may be used to prepare substituted acetylaniline intermediates S2. Treatment of S2 with a base such as sodium hydride in an organic solvent such as THF and treatment with an acyl chloride S3 yields intermediates of formula S4. Quinolones like S5 may then be prepared from S4 by treatment with base such as sodium hydroxide in an organic solvent such as dioxane at elevated temperature.

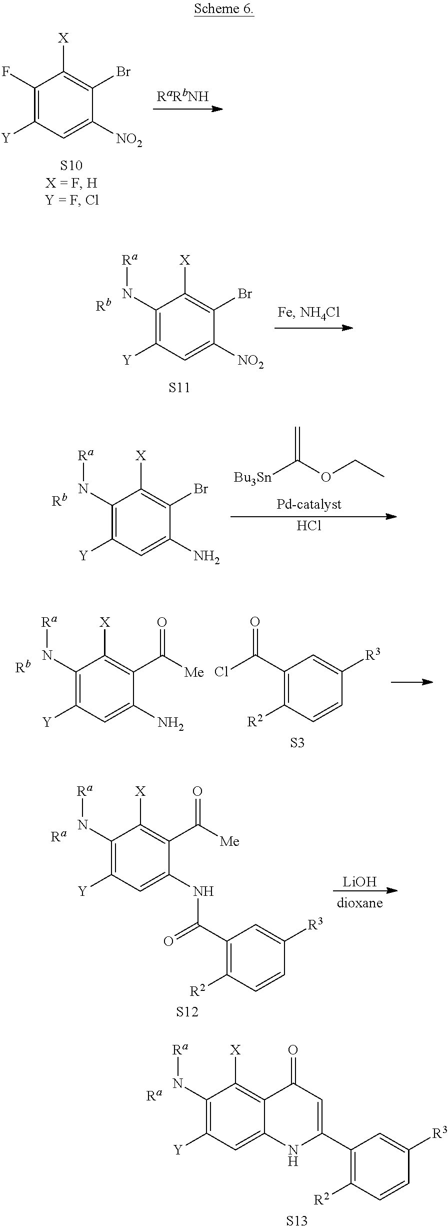

Quinolones like S13 may be prepared by the general synthetic methods as shown in Scheme 6. Treatment of S10 with an amine provides intermediate S11 which can be converted to intermediate S12 and quinolone S13 using methods described herein.

Preparation of Starting Materials

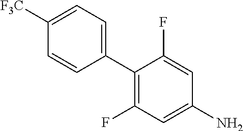 [SMILES: Nc1cc(F)c(-c2ccc(C(F)(F)F)cc2)c(F)c1]

### 2,6-difluoro-4′-(trifluoromethyl)-[1,1′-biphenyl]-4-amine

To a solution of 4-bromo-3,5-difluoro-aniline (1.0 g, 4.8 mmol, 1.0 equiv.) and 4-(trifluoromethyl)phenyl]boronic acid (1.37 g, 7.21 mmol, 1.5 1.0 equiv.) in dioxane (9 mL) and H₂O (3 mL) was added Pd(dppf)Cl₂·CH₂Cl₂ (392 mg, 480 μmol, 0.1 eq) and K₂CO₃ (1.99 g, 14.4 mmol, 3.0 1.0 equiv.). The mixture was stirred at 80° C. for 16 hours. The reaction mixture was diluted with ethyl acetate (50 mL) and washed with H₂O (20 mL) and brine (20 mL). The organic layer was dried over Na₂SO₄, filtered, and concentrated under reduced pressure. The residue was purified by column chromatography (SiO₂, petroleum ether:ethyl acetate=10:1 to 5:1) to afford the title compound (1.2 g, 91% yield) as a white solid. LCMS: calculated for [M+H]⁺ (Cl₃H₈F₅N) requires m/z=274.1, found m/z=274.0. ¹H NMR (400 MHz, CDCl₃) δ 7.67 (d, J=8.2 Hz, 2H), 7.55 (d, J=8.2 Hz, 2H), 6.34-6.27 (m, 2H), 4.00 (br s, 2H).

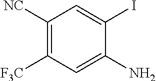 [SMILES: N#Cc1cc(I)c(N)cc1C(F)(F)F]

### 4-amino-5-iodo-2-(trifluoromethyl) benzonitrile

To a solution of 4-amino-2-(trifluoromethyl) benzonitrile (1.0 g, 5.37 mmol, 1.0 equiv.) in THF (10 mL) and MeOH (10 mL) was added NIS (1.2 g, 5.37 mmol, 1.0 equiv.) and 4-methylbenzenesulfonic acid-hydrate (1.02 g, 5.37 mmol, 1.0 1.0 equiv.). The mixture was stirred at 20° C. for 16 hours. The reaction was concentrated under reduced pressure, and the residue was extracted with ethyl acetate (3×20 mL) and H₂O (20 mL). The combined organic layers were washed with brine (20 mL), dried over anhydrous Na₂SO₄, filtered and concentrated under reduced pressure. The residue was purified by column chromatography (SiO₂, petroleum ether:EtOAc=100:1 to 10:1) to afford the title compound (1.3 g, 78% yield) as a yellow solid. LCMS: mass calculated for [M+H]⁺ (C₈H₄F₃IN₂) requires m/z=312.9, found m/z=312.9. ¹H NMR (400 MHz, CDCl₃) δ 8.05 (s, 1H), 6.99 (s, 1H), 4.89 (br s, 2H).

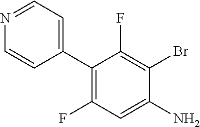 [SMILES: Nc1cc(F)c(-c2ccncc2)c(F)c1Br]

### 2-bromo-3,5-difluoro-4-(pyridin-4-yl)aniline

### Step 1, 3,5-difluoro-4-(pyridin-4-yl)aniline

This material was prepared in a similar fashion to that described for 2,6-difluoro-4′-(trifluoromethyl)-[1,1′-biphenyl]-4-amine using 4-pyridylboronic acid as the starting material. ¹H NMR (400 MHz, CHLOROFORM-d) δ 8.67-8.61 (m, 2H), 7.38 (dd, J=1.6, 4.6 Hz, 2H), 6.38-6.23 (m, 2H), 4.07 (br s, 2H).

### Step, 2. 2-bromo-3,5-difluoro-4-(pyridin-4-yl)aniline

To a solution of 3,5-difluoro-4-(pyridin-4-yl)aniline (850 mg, 4.1 mmol, 1.0 equiv.) in THF (10 mL) was added N-bromosuccinimide (660 mg, 3.7 mmol, 0.9 equiv.) at −5° C. The mixture was stirred at −5° C. for 16 hours. The reaction mixture was diluted with ethyl acetate (50 mL). The organic layer was washed with water (20 mL) and brine (20 mL), dried over Na₂SO₄, filtered and concentrated under reduced pressure to give a residue. Purification by silica gel chromatography (3:1 to 1:1 petroleum ether:ethyl acetate) afforded the title compound (1.0 g, 85.1% yield) as a light yellow solid.

¹H NMR (400 MHz, CHLOROFORM-d) δ 8.67 (d, J=5.0 Hz, 2H), 7.41-7.33 (m, 2H), 6.45 (dd, J=1.8, 11.4 Hz, 1H), 4.56 (br s, 2H).

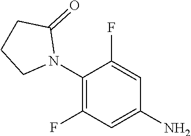 [SMILES: Nc1cc(F)c(N2CCCC2=O)c(F)c1]

### 1-(4-amino-2,6-difluorophenyl)pyrrolidin-2-one

### Step 1, 1-(2,6-difluoro-4-nitrophenyl)pyrrolidin-2-one

To a mixture of pyrrolidin-2-one (1.43 mL, 18.6 mmol, 1.1 equiv.) in DMF (10 mL) was added NaH (1.22 g, 30.4 mmol, 1.8 equiv.; 60% dispersion) at 0° C. under N₂. The mixture was stirred at 0° C. for 20 minutes, then to the mixture was added 1,2,3-trifluoro-5-nitrobenzene (3.0 g, 16.9 mmol, 1 equiv.) at 0° C. The mixture was stirred at 0° C. and stirred for 3 hours. The reaction mixture was poured into saturated aqueous NH₄Cl solution (20 mL) and extracted with ethyl acetate (2×40 mL). The combined organic layers were washed with brine (2×20 mL), dried with anhydrous Na₂SO₄, filtered, and concentrated under reduced pressure. The crude product was purified by silica gel column chromatography (10:1 to 5:1 petroleum ether:ethyl acetate) to afford the title compound (3.5 g, 85% yield) as light yellow solid. ¹H NMR (400 MHz, METHANOL-d₄) δ 8.09-8.01 (m, 2H), 3.87 (t, J=7.0 Hz, 2H), 2.62-2.55 (m, 2H), 2.32 (quin, J=7.6 Hz, 2H).

### Step 2, 1-(4-amino-2,6-difluorophenyl)pyrrolidin-2-one

To a mixture of 1-(2,6-difluoro-4-nitrophenyl)pyrrolidin-2-one (1.5 g, 6.19 mmol, 1.0 equiv.) in EtOH (4 mL) and water (1 mL) was added iron(0) (1.73 g, 30.9 mmol, 5.0 equiv.) and NH₄Cl (1.66 g, 30.9 mmol, 5.0 equiv.) at 20° C. under N₂. The mixture was stirred at 80° C. for 2 hours. The reaction mixture was filtered and concentrated under reduced pressure to give a crude product. The crude product was poured into water (20 mL) and the aqueous phase was extracted with ethyl acetate (3×30 mL). The combined organic layers were washed with brine (2×20 mL), dried with anhydrous Na₂SO₄, filtered, and concentrated under reduced pressure to afford the title compound (950 mg, 72% yield) as yellow solid. ¹H NMR (400 MHz, METHANOL-d₄) δ 6.36-6.22 (m, 2H), 3.74-3.65 (m, 2H), 2.57-2.48 (m, 2H), 2.23 (quin, J=7.6 Hz, 2H).

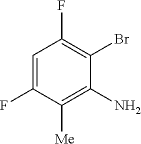 [SMILES: Cc1c(F)cc(F)c(Br)c1N]

### 2-bromo-3,5-difluoro-6-methylaniline

### Step 1, N-(4,6-difluoro-3-methyl-2-nitrophenyl)acetamide

To a mixture of N-(2,4-difluoro-5-methylphenyl)acetamide (1.5 g, 8.1 mmol, 1.0 equiv.) in H₂SO₄ (15 mL) was added dropwise a mixture of HNO₃ (1.1 mL, 24.3 mmol, 3.0 equiv.) and H₂SO₄ (1.5 mL) at 0° C. under N₂. The mixture was stirred at 20° C. for 1 hour. The reaction mixture was poured into ice-cold water (40 mL) and the solid that precipitated was filtered and washed with water (2×100 mL). The solid residue was dissolved in ethyl acetate (300 mL) and washed with aqueous NaHCO₃(2×40 mL). The organic layer was dried over Na₂SO₄, filtered, and concentrated under reduced pressure. The residue was purified by silica gel column chromatography (100:1 to 20:1 petroleum ether:ethyl acetate) to afford the title compound (1.4 g, 75% yield) as yellow solid. LCMS [M+1]=231.0.

### Step 2, 4,6-difluoro-3-methyl-2-nitroaniline

To a mixture of N-(4,6-difluoro-3-methyl-2-nitrophenyl)acetamide (1.4 g, 6.1 mmol, 1.0 equiv.) in MeOH (5.0 mL) was added dropwise HCl (12 M, 9.8 mL, 19.3 equiv.) at 20° C. under N₂. The mixture was stirred at 90° C. for 16 hours. The residue was poured into ice-water (100 mL) and extracted with ethyl acetate (2×100 mL). The combined organic layers were washed with brine (2×50 mL), dried with anhydrous Na₂SO₄, filtered, and concentrated under reduced pressure. The residue was purified by silica gel column chromatography (50:1 to 10:1 petroleum ether:ethyl acetate) to afford the title compound (1.1 g, 92% yield) as yellow solid. ¹H NMR (400 MHz, CHLOROFORM-d) δ 7.01 (dd, J=8.8, 10.2 Hz, 1H), 4.99 (br s, 2H), 2.34 (dd, J=1.2, 2.4 Hz, 3H).

### Step 3, 2-bromo-1,5-difluoro-4-methyl-3-nitrobenzene

To a mixture of 4,6-difluoro-3-methyl-2-nitroaniline (1.1 g, 5.85 mmol, 1.0 equiv.) and CuBr₂ (2.61 g, 11.7 mmol, 548 uL, 2.0 equiv.) in MeCN (15 mL) was added tert-butyl nitrite (2.78 mL, 23.4 mmol, 4.0 equiv.) in one portion at 20° C. under N₂. The mixture was stirred at 20° C. for 16 hours. The mixture was filtered and concentrated under reduced pressure and then poured into DCM and filtered. The filtrate was concentrated in vacuum. The residue was purified by silica gel column chromatography (50:1 to 10:1 petroleum ether:ethyl acetate) to afford the title compound (1.3 g, 88% yield) as yellow solid. ¹H NMR (400 MHz, CHLOROFORM-d) δ 7.06 (t, J=8.4 Hz, 1H), 2.28-2.22 (m, 3H).

### Step 4, 2-bromo-3,5-difluoro-6-methylaniline

To a mixture of 2-bromo-1,5-difluoro-4-methyl-3-nitrobenzene (1.3 g, 5.2 mmol, 1.0 equiv.) in EtOH (13 mL) and water (6.5 mL) was added NH₄Cl (1.4 g, 25.8 mmol, 5.0 equiv.) and iron(0) (2.0 g, 36.1 mmol, 7.0 equiv.) in one portion at 20° C. under N₂. The mixture was stirred at 80° C. for 2 hours. The mixture was filtered and concentrated under reduced pressure. The residue was purified by silica gel column chromatography (50:1 to 20:1 petroleum ether:ethyl acetate) to afford the title compound (650 mg, 57% yield) as white solid. LCMS [M+1]=223.8. ¹H NMR (400 MHz, CHLOROFORM-d) δ 6.35 (t, J=9.2 Hz, 1H), 4.33 (br s, 2H), 2.09 (s, 3H).

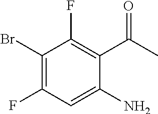

### 1-(6-amino-3-bromo-2,4-difluorophenyl)ethan-1-one

### Step 1, 1-(2-amino-4,6-difluorophenyl)ethan-1-one

To a solution of BCl₃ (55.4 mL, 426 mmol, 1.1 eq) was added a solution of 3,5-difluoroaniline (49.9 g, 426 mmol, 1.1 eq) in DCE (850 mL) at 0° C. dropwise over 15 minutes, maintaining a temperature between 0-10° C. To the mixture was added acetonitrile (61.14 mL, 1.16 mol, 3.0 equiv.) dropwise over 2-3 minutes and then AlCl₃ (56.80 g, 426.00 mmol, 1.1 equiv.) in portions over 10 minutes, maintaining a temperature between 0-10° C. during the addition of both reagents. Then the mixture was refluxed at 80° C. for 16 hours. Upon completion of the reaction, the reaction mixture was cooled to 0° C., before quenching the reaction with 4 N HCl (500 mL). The mixture was stirred at 80° C. for 2 hours. The reaction mixture was extracted with DCM (3×400 mL). The combined organic layers were washed with saturated aqueous NaHCO₃ (500 mL) and brine (500 mL), dried over anhydrous Na₂SO₄, and concentrated under reduced pressure to afford the title compound (45 g, 67.7% yield, 99.7% purity) as an off-white solid. LCMS [M+1]=172.1. ¹H NMR (400 MHz, CDCl₃) δ 6.3-6.7 (m, 2H), 6.1-6.2 (m, 2H), 2.6-2.6 (m, 3H).

### Step 2, 1-(6-amino-3-bromo-2,4-difluorophenyl)ethan-1-one

To a mixture of 1-(2-amino-4,6-difluoro-phenyl)ethanone (40 g, 234 mmol, 1.0 equiv.) in DCM (400 mL) was added N-bromosuccinimide (45.8 g, 257 mmol, 1.1 equiv.) at 20° C. under N₂. The mixture was stirred at 20° C. for 2 hours. The reaction mixture was poured into water (50 mL) and the aqueous layer was extracted with DCM (3×200 mL). The combined organic layers were washed with brine (2×50 mL), dried with anhydrous Na₂SO₄, filtered, and concentrated under reduced pressure. The crude product was purified by silica gel column chromatography (20:1 to 12:1 petroleum ether:ethyl acetate) to afford the title compound (29.9 g, 47% yield) as yellow solid. LCMS [M+1]=249.9/251.9. ¹H NMR (400 MHz, CHLOROFORM-d) δ 6.71-6.46 (m, 2H), 6.26 (dd, J=1.8, 10.2 Hz, 1H), 2.60 (d, J=8.6 Hz, 3H).

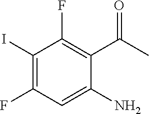

1-(6-amino-2,4-difluoro-3-iodo-phenyl)ethenone: To a solution of 1-(2-amino-4,6-difluoro-phenyl)ethanone (10.0 g, 58.4 mmol, 1.0 equiv.) in DCM (100 mL) was added N-iodosuccinimide (14.4 g, 64.2 mmol, 1.1 equiv.). The mixture was stirred at 20° C. for 16 hours. The reaction mixture was diluted with water (100 mL) and extracted with DCM (2×200 mL). The combined organic layers were washed with brine 300 mL, dried over Na₂SO₄, filtered, and concentrated under reduced pressure to give a residue. The residue was purified by silica gel column chromatography (0-33% ethyl acetate in petroleum ether) to afford the title compound (6.5 g, 37% yield) as a brown solid. LCMS [M+1]=297.7. ¹H NMR (400 MHz, CHLOROFORM-d) δ 6.70-6.43 (m, 2H), 6.26 (dd, J=1.8, 9.6 Hz, 1H), 2.61 (d, J=9.0 Hz, 3H).

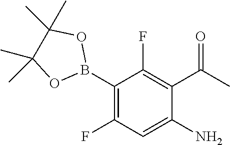 [SMILES: CC(=O)c1c(N)cc(F)c(B2OC(C)(C)C(C)(C)O2)c1F]

1-[6-amino-2,4-difluoro-3-(4,4,5,5-tetramethyl-1,3,2-dioxaborolan-2-yl)phenyl]ethenone: To a mixture of 1-(6-amino-3-bromo-2,4-difluoro-phenyl)ethanone (10 g, 39.99 mmol, 1 equiv.) and 4,4,5,5-tetramethyl-2-(4,4,5,5-tetramethyl-1,3,2-dioxaborolan-2-yl)-1,3,2-dioxaborolane (30.47 g, 119.98 mmol, 3 equiv.) in toluene (200 mL) was added KOAc (7.85 g, 79.99 mmol, 2 equiv.) and [2-(2-aminophenyl)phenyl]-chloro-palladium; dicyclohexyl-[3-(2,4,6-triisopropylphenyl)phenyl]phosphane (944.01 mg, 1.20 mmol, 0.03 equiv.) at 20° C. under N₂. The mixture was stirred at 80° C. for 5 hours. The reaction mixture was filtered and concentrated under reduced pressure to give the crude product which was purified by silica gel column chromatography (50:1 to 15:1 petroleum ether:ethyl acetate) to afford the title compound (5.5 g, 46.1% yield) as white solid. LCMS [M+1]=298.1 and 216.1 (boric acid MS). ¹H NMR (400 MHz, METHANOL-d₄) δ 6.21 (dd, J=1.2, 11.6 Hz, 1H), 2.53 (d, J=8.8 Hz, 3H), 1.33 (s, 12H).

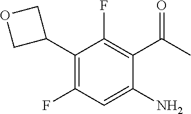 [SMILES: CC(=O)c1c(N)cc(F)c(C2COC2)c1F]

1-(6-amino-2,4-difluoro-3-(oxetan-3-yl)phenyl)ethan-1-one: To a solution of 1-(6-amino-2,4-difluoro-3-iodophenyl)ethan-1-one (200 mg, 673 μmol, 1.0 equiv.) and 3-iodooxetane (124 mg, 673 μmol, 1.0 equiv.) in DME (2 mL) was added NiCl₂·glyme (740 ug, 3.3 μmol, 0.005 equiv.), Na₂CO₃ (143 mg, 1.3 mmol, 2 equiv.), 4,4′-di-tert-butyl-2,2′-dipyridyl (903 ug, 3.3 μmol, 0.005 equiv.), tris(trimethylsilyl)silane (208 uL, 673 μmol, 1.0 equiv.) and bis[3,5-difluoro-2-[5-(trifluoromethyl)-2-pyridyl]phenyl]iridium(1+)-4-tert-butyl-2-(4-tert-butyl-2pyridyl)pyridine hexafluorophosphate (7.6 mg, 6.7 μmol, 0.01 equiv.). The mixture was stirred at 20° C. for 2 hours. The residue was diluted with water (10 mL) and the aqueous layer was extracted with ethyl acetate (3×15 mL). The combined organic layers were washed with brine (5 mL), dried over anhydrous Na₂SO₄, filtered, and concentrated under reduced pressure. The residue was purified by silica gel column chromatography (5:1 to 1:1, petroleum ether:ethyl acetate) to afford the title compound (120 mg, 78% yield) as a white solid. ¹H NMR (400 MHz, CHLOROFORM-d) δ 6.60-6.23 (m, 1H), 6.16 (dd, J=1.8, 12.1 Hz, 1H), 5.07-4.96 (m, 2H), 4.96-4.86 (m, 2H), 4.63-4.42 (m, 1H), 2.58 (d, J=8.8 Hz, 3H).

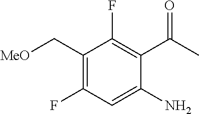

1-(6-amino-2,4-difluoro-3-(methoxymethyl)phenyl)ethan-1-one: To a solution of 1-(6-amino-2,4-difluoro-3-iodophenyl)ethan-1-one (1.5 g, 5.0 mmol, 1.0 equiv.) and tributyl(methoxymethyl)stannane (5.0 g, 15.1 mmol, 3.0 equiv.) in DMF (2 mL) was added [2-(2-aminophenyl)phenyl]-chloro-palladium-dicyclohexyl-[3-(2,4,6-triisopropylphenyl)phenyl]phosphane (397 mg, 505 μmol, 0.1 equiv.). The mixture was stirred at 100° C. for 16 hours. The reaction was diluted with water and the aqueous mixture was extracted with ethyl acetate (3×20 mL). The combined organic layers were washed with brine (5 mL), dried over anhydrous Na₂SO₄, filtered, and concentrated under reduced pressure. The residue was purified by silica gel column chromatography (10:1 to 2:1 petroleum ether:ethyl acetate) to afford the title compound (770 mg, 71% yield) as a white solid. ¹H NMR (400 MHz, CHLOROFORM-d) δ 6.69-6.37 (m, 2H), 6.16 (dd, J=1.8, 11.2 Hz, 1H), 4.48-4.39 (m, 2H), 3.43-3.33 (m, 3H), 2.60 (d, J=8.8 Hz, 3H).

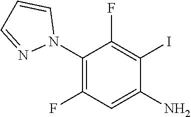 [SMILES: Nc1cc(F)c(-n2cccn2)c(F)c1I]

### 3,5-difluoro-2-iodo-4-(1H-pyrazol-1-yl)aniline

### Step 1, 1-(2,6-difluoro-4-nitrophenyl)-1H-pyrazole

To a mixture of 3, 4, 5-trifluoronitrobenzne (2 g, 11.3 mmol, 1.0 equiv.) and 1H-pyrazole (768.9 mg, 11.3 mmol, 1.0 equiv.) in DMSO (15 mL) was added K₂CO₃ (2.34 g, 16.9 mmol, 1.5 equiv.) at 20° C. under N₂. The mixture was stirred at 20° C. for 16 hours. The mixture was poured into water (100 mL) and extracted with ethyl acetate (3×100 mL). The combined organic layers were washed with brine (3×100 mL), dried with Na₂SO₄, filtered, and concentrated under reduced pressure. The residue was triturated with MTBE (3×50 mL) to afford the title compound (1.7 g, 67% yield) as a white solid. ¹H NMR (400 MHz, DMSO-d₆) δ 8.39-8.32 (m, 2H), 8.22 (s, 1H), 7.91 (d, J=1.6 Hz, 1H), 6.65 (t, J=2.2 Hz, 1H).

### Step 2, 3,5-difluoro-4-(1H-pyrazol-1-yl)aniline

To a mixture of 1-(2,6-difluoro-4-nitrophenyl)-1H-pyrazole (500 mg, 2.22 mmol, 1.0 equiv.) in EtOH (5 mL) and water (1 mL) was added NH₄Cl (594 mg, 11.1 mmol, 5.0 equiv.) and iron(0) (620 mg, 11.1 mmol, 5.0 equiv.) at 20° C. under N₂. The mixture was stirred at 80° C. for 2 hours. The reaction mixture was filtered and concentrated under reduced pressure. The residue was washed with water (3×10 mL) to afford the title compound (600 mg, crude) as yellow solid. ¹H NMR (400 MHz, METHANOL-d₄) δ 7.71 (t, J=2.0 Hz, 2H), 6.49 (t, J=2.0 Hz, 1H), 6.39-6.27 (m, 2H).

### Step 3, 3,5-difluoro-2-iodo-4-(1H-pyrazol-1-yl)aniline

To a mixture of 3,5-difluoro-4-(1H-pyrazol-1-yl)aniline (320 mg, 1.64 mmol, 1.0 equiv.) in acetic acid (1 mL) was added N-iodosuccinimide (350 mg, 1.56 mmol, 0.95 equiv.) at 20° C. under N₂. The mixture was stirred at 20° C. for 2 hours. The residue was poured into a saturated aqueous solution of NaHCO₃ (20 mL). The aqueous mixture was extracted with ethyl acetate (2×40 mL). The combined organic layers were washed with brine (2×20 mL), dried with anhydrous Na₂SO₄, filtered, and concentrated under reduced pressure. The residue was purified by silica gel column chromatography (10:1 to 5:1 petroleum ether:ethyl acetate) to afford the title compound (450 mg, 85.5% yield) as a light yellow solid. ¹H NMR (400 MHz, DMSO-d₆) δ 7.93 (d, J=2.4 Hz, 1H), 7.70 (d, J=1.8 Hz, 1H), 6.77-6.51 (m, 1H), 6.46 (t, J=2.2 Hz, 1H), 6.19 (s, 2H).

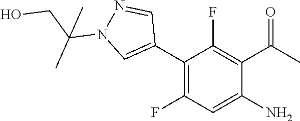 [SMILES: CC(=O)c1c(N)cc(F)c(-c2cnn(C(C)(C)CO)c2)c1F]

1-(6-amino-2,4-difluoro-3-(1-(1-hydroxy-2-methylpropan-2-yl)-1H-pyrazol-4-yl)phenyl)ethan-1-one: To a solution of 2-(4-bromo-1H-pyrazol-1-yl)-2-methylpropan-1-ol (885 mg, 4.0 mmol, 2 equiv.) and 1-(6-amino-2,4-difluoro-3-(4,4,5,5-tetramethyl-1,3,2-dioxaborolan-2-yl)phenyl)ethan-1-one (600 mg, 2.0 mmol, 1 equiv.) in dioxane (6 mL) and water (2 mL) was added K₂CO₃ (837 mg, 6.0 mmol, 3.0 equiv.) and Pd(dppf)Cl₂—CH₂Cl₂ (165 mg, 202 μmol, 0.1 equiv.). The mixture was stirred at 80° C. for 16 hours. The reaction was diluted with water (20 mL) and the mixture was extracted with ethyl acetate (3×20 mL). The combined organic layers were washed with brine (20 mL), dried over Na₂SO₄, filtered, and concentrated under reduced pressure. The residue was purified by silica gel column chromatography (1:1 petroleum ether:Ethyl acetate) to afford the title compound (160 mg, 26% yield) as a brown solid. ¹H NMR (400 MHz, CHLOROFORM-d) δ 7.84 (br d, J=6.8 Hz, 2H), 7.56-7.45 (m, 1H), 6.52-6.32 (m, 2H), 6.30-6.20 (m, 1H), 3.85 (s, 2H), 2.64 (d, J=8.8 Hz, 3H), 1.61 (s, 6H).

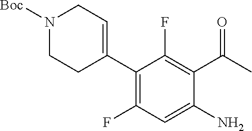 [SMILES: CC(=O)c1c(N)cc(F)c(C2=CCN(C(=O)OC(C)(C)C)CC2)c1F]

tert-butyl 4-(3-acetyl-4-amino-2,6-difluorophenyl)-3,6-dihydropyridine-1(2H)-carboxylate: This material was prepared in a similar fashion to that described for 3′-acetyl-4′-amino-2′,6′-difluoro-[1,1′-biphenyl]-4-carbonitrile. ¹H NMR (400 MHz, CHLOROFORM-d) δ 6.17 (d, J=1.8 Hz, 1H), 5.73 (br s, 1H), 4.06 (q, J=2.8 Hz, 2H), 3.62 (t, J=5.6 Hz, 2H), 2.57 (d, J=8.8 Hz, 3H), 2.36 (br s, 2H), 1.50 (s, 9H).

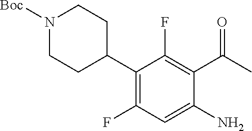 [SMILES: CC(=O)c1c(N)cc(F)c(C2CCN(C(=O)OC(C)(C)C)CC2)c1F]

tert-butyl 4-(3-acetyl-4-amino-2,6-difluorophenyl)piperidine-1-carboxylate: To a solution of tert-butyl 4-(3-acetyl-4-amino-2,6-difluorophenyl)-3,6-dihydropyridine-1(2H)-carboxylate (500 mg, 1.4 mmol, 1 equiv.) in MeOH (5 mL) was added 10% Pd/C (100 mg, 100 μmol, 0.07 equiv.) at 20° C. under N₂. The suspension was degassed under vacuum and purged with H₂ several times. The mixture was stirred under an atmosphere of H₂ (15 psi) at 20° C. for 2 hours. The suspension was filtered through a pad of celite and the filter cake was washed with MeOH (3×10 mL). Concentration of the filtrate under reduced pressure afforded the title compound (500 mg, crude) as a brown solid. ¹H NMR (400 MHz, CHLOROFORM-d) δ 6.12 (dd, J=1.6, 12.4 Hz, 1H), 4.23 (br d, J=12.4 Hz, 2H), 2.99 (tt, J=3.4, 12.4 Hz, 1H), 2.76 (br t, J=12.6 Hz, 2H), 2.57 (d, J=9.1 Hz, 3H), 2.05-1.88 (m, 2H), 1.65 (br d, J=13.4 Hz, 2H), 1.54-1.43 (m, 9H).

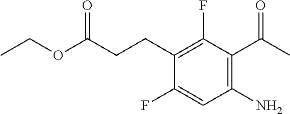 [SMILES: CCOC(=O)CCc1c(F)cc(N)c(C(C)=O)c1F]

### Ethyl 3-(3-acetyl-4-amino-2,6-difluorophenyl)propanoate

### Step 1, ethyl (E)-3-(3-acetyl-4-amino-2,6-difluorophenyl)acrylate

To a solution of 1-(6-amino-3-bromo-2,4-difluorophenyl)ethan-1-one (0.9 g, 3.60 mmol, 1.0 equiv.) and ethyl (E)-3-(4,4,5,5-tetramethyl-1,3,2-dioxaborolan-2-yl)acrylate (2.03 g, 9.00 mmol, 2.5 equiv.) in dioxane (12 mL) and water (4 mL) was added Pd(dppf)Cl₂—CH₂Cl₂ (294 mg, 360 μmol, 0.1 equiv.) and K₂CO₃ (1.49 g, 10.8 mmol, 3.0 equiv.). The mixture was stirred at 100° C. for 8 hours under N₂. The reaction mixture was poured into water (20 mL) and the aqueous phase was extracted with ethyl acetate (2×30 mL). The combined organic layers were washed with brine (10 mL), dried over Na₂SO₄, filtered, and concentrated under reduced pressure. The residue was purified by silica gel chromatography (10:1 to 3:1 petroleum ether:ethyl acetate) to afford the title compound (0.7 g, 72% yield) as a brown solid. ¹H NMR (400 MHz, DMSO-d₆) δ 8.11-7.82 (m, 2H), 7.52 (d, J=16.4 Hz, 1H), 6.51 (d, J=13.8 Hz, 1H), 6.34 (d, J=16.4 Hz, 1H), 4.18 (q, J=7.2 Hz, 2H), 2.54 (s, 3H), 1.25 (t, J=7.0 Hz, 3H).

### Step 2, ethyl 3-(3-acetyl-4-amino-2,6-difluorophenyl)propanoate

To a solution of ethyl (E)-3-(3-acetyl-4-amino-2,6-difluorophenyl)acrylate (700 mg, 2.60 mmol, 1.0 equiv.) in EtOH (5 mL) and THF (5 mL) was added 10% Pd/C (100 mg). The mixture was stirred at 20° C. for 16 hours under an atmosphere of H₂ (15 Psi). The reaction mixture was filtered and concentrated. The residue was purified by silica gel chromatography (10:1 to 3:1 petroleum ether:ethyl acetate) to afford the title compound (600 mg, 85% yield) as a white solid. LCMS [M+1]=272.1. ¹H NMR (400 MHz, DMSO-d₆) δ 7.43 (br s, 2H), 6.47-6.28 (m, 1H), 4.04 (q, J=7.2 Hz, 2H), 2.84-2.67 (m, 2H), 2.48 (d, J=8.6 Hz, 5H), 1.16 (t, J=7.2 Hz, 3H).

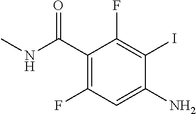

### 4-amino-2,6-difluoro-3-iodo-N-methylbenzamide

### Step 1, methyl 4-amino-2,6-difluoro-3-iodobenzoate

N-iodosuccinimide (4.8 g, 21.4 mmol, 1.0 equiv) was added to a solution of methyl 4-amino-2,6-difluorobenzoate (3.8 g, 20.4 mmol, 1.0 equiv.) in acetic acid (40 mL) at 20° C. Then the reaction mixture was stirred at 10° C. for 0.5 hours. The mixture was quenched with a saturated aqueous solution of Na₂S₂O₃ (50 mL). The aqueous layer was extracted with ethyl acetate. The organic extract was dried over Na₂SO₄, filtered and concentrated to afford the title compound (5.5 g, crude) as a white solid. ¹H NMR (400 MHz, DMSO-d₆) δ 6.60-6.49 (m, 2H), 6.43 (dd, J=1.4, 13.3 Hz, 1H), 3.77 (s, 3H).

### Step 2, 4-amino-2,6-difluoro-3-iodo-N-methylbenzamide

The solution of methyl 4-amino-2,6-difluoro-3-iodobenzoate (1 g, 3.1 mmol, 1 equiv.) in methylamine (22.0 mL, 319 mmol, 100 equiv.; 40% solution in water) was stirred at 20° C. for 1 hour. The mixture was diluted with H₂O (15 mL) and extracted with EA (2×20 mL). The combined organic layers were washed with brine (2×15 mL), dried with anhydrous Na₂SO₄, filtered and concentrated under reduced pressure to give a residue. The residue was purified by silica gel column chromatography (80:1 to 20:1 petroleum ether:ethyl acetate) to afford the title compound (800 mg, 80% yield) as a yellow solid. ¹H NMR (400 MHz, METHANOL-d₄) δ 6.45-6.37 (m, 1H), 2.87 (s, 3H).

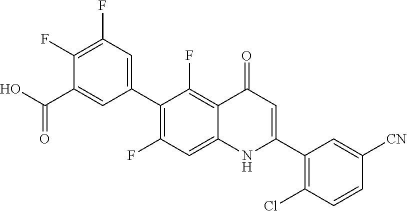 [SMILES: N#Cc1ccc(Cl)c(-c2cc(=O)c3c(F)c(-c4cc(F)c(F)c(C(=O)O)c4)c(F)cc3[nH]2)c1]

### 5-(2-(2-chloro-5-cyanophenyl)-5,7-difluoro-4-oxo-1,4-dihydroquinolin-6-yl)-2,3-difluorobenzoic acid

### Step 1, methyl 3′-acetyl-4′-amino-2′,4,5,6′-tetrafluoro-[1,1′-biphenyl]-3-carboxylate

To a solution of methyl 5-bromo-2,3-difluorobenzoate (500 mg, 2.0 mmol, 1 equiv.), 1-(6-amino-2,4-difluoro-3-(4,4,5,5-tetramethyl-1,3,2-dioxaborolan-2-yl)phenyl)ethan-1-one (888 mg, 3.0 mmol, 1.5 equiv.) and K₃PO₄ (846 mg, 3.9 mmol, 2 equiv.) in water (2 mL) and THF (8 mL), was added [1,1′-bis(di-tert-butylphosphino)ferrocene]dichloropalladium(II) (129.8 mg, 199 μmol, 0.1 equiv.). The reaction mixture was stirred at 80° C. for 2 hours. The reaction mixture was poured into water (5 mL) and stirred for 2 min. The aqueous phase was extracted with ethyl acetate (2×5 mL). The combined organic layers were washed with brine (2×5 mL), dried over Na₂SO₄, filtered and concentrated under reduced pressure. The residue was purified by silica gel column chromatography (15:1 to 5:1 petroleum ether:ethyl acetate) to afford the title compound (400 mg, 59% yield) as white solid. ¹H NMR (400 MHz, CHLOROFORM-d) δ ppm 7.72-7.78 (m, 1H) 7.38-7.46 (m, 1H) 6.26 (dd, J=11.6, 1.53 Hz, 1H) 3.93-4.00 (m, 3H) 2.60 (d, J=8.8 Hz, 3H).

### Step 2, methyl 3′-acetyl-4′-(2-chloro-5-cyanobenzamido)-2′,4,5,6′-tetrafluoro-[1,1′-biphenyl]-3-carboxylate

To a mixture of methyl 3′-acetyl-4′-amino-2′,4,5,6′-tetrafluoro-[1,1′-biphenyl]-3-carboxylate (240 mg, 703 μmol, 1.0 equiv.) in THF (2 mL) was added NaH (28.1 mg, 703 μmol, 1.0 equiv.; 60% dispersion in oil) at 0° C. under N₂. The mixture was stirred at 20° C. for 2 minutes before the addition of 2-chloro-5-cyanobenzoyl chloride (168.8 mg, 843.9 μmol, 1.2 equiv.) under N₂. The mixture was stirred at 20° C. for 16 hours. The reaction mixture was poured into a saturated aqueous solution of NH₄Cl (20 mL). The aqueous phase was extracted with ethyl acetate (2×30 mL). The combined organic layer were washed with brine (2×30 mL), dried over Na₂SO₄, filtered, and concentrated under reduced pressure. The crude product was triturated with ethyl acetate (3×1 mL) at 20° C. for 2 min, and filtered and concentrated under reduced pressure to afford the title compound (120 mg, 237.7 μmol, 33.8% yield) was obtained as white solid. ¹H NMR (400 MHz, DMSO-d₆) δ=11.23 (s, 1H), 8.13 (d, J=1.8 Hz, 1H), 8.06 (dd, J=2.0, 8.4 Hz, 2H), 7.94-7.81 (m, 2H), 7.64-7.52 (m, 1H), 3.91 (s, 3H), 2.58 (d, J=3.8 Hz, 3H).

### Step 3, 5-(2-(2-chloro-5-cyanophenyl)-5,7-difluoro-4-oxo-1,4-dihydroquinolin-6-yl)-2,3-difluorobenzoic acid

To a mixture of methyl 3′-acetyl-4′-(2-chloro-5-cyanobenzamido)-2′,4,5,6′-tetrafluoro-[1,1′-biphenyl]-3-carboxylate (110 mg, 217.9 μmol, 1 equiv.) in dioxane (3 mL) was added LiGH (10.4 mg, 435.8 μmol, 2 equiv.) at 20° C. under N₂. The mixture was stirred at 110° C. for 2 hours. Additional LiGH (5.2 mg, 217.9 μmol, 1 equiv.) was added and the reaction was stirred at 110° C. for another 2 hours. The reaction mixture was poured into water (5 mL) and the 2aqueous phase was extracted with ethyl acetate (2×5 mL). The combined organic layers were washed with brine (2×5 mL), dried over Na₂SO₄, filtered, and concentrated under reduced pressure. The crude product was triturated with ethyl acetate (3×1 mL), filtered, and concentrated under reduced pressure to afford the title compound (65 mg, 67% yield) as white solid. ¹H NMR (400 MHz, DMSO-d₆) δ ppm 8.22 (t, J=2.6 Hz, 1H) 8.02-8.10 (m, 1H) 7.90 (d, J=8.4 Hz, 1H) 7.39-7.50 (m, 3H) 7.26-7.32 (m, 1H).

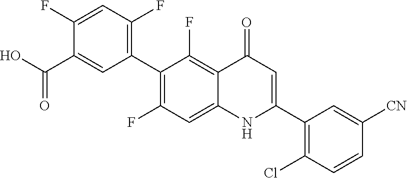 [SMILES: N#Cc1ccc(Cl)c(-c2cc(=O)c3c(F)c(-c4cc(C(=O)O)c(F)cc4F)c(F)cc3[nH]2)c1]

5-(2-(2-chloro-5-cyanophenyl)-5,7-difluoro-4-oxo-1,4-dihydroquinolin-6-yl)-2,4-difluorobenzoic acid: This material was prepared using the same synthetic sequence that was described for 5-(2-(2-chloro-5-cyanophenyl)-5,7-difluoro-4-oxo-1,4-dihydroquinolin-6-yl)-2,3-difluorobenzoic acid. ¹H NMR (400 MHz, METHANOL-d₄) δ 8.20 (d, J=2.2 Hz, 1H), 8.16-8.08 (m, 1H), 7.98-7.76 (m, 3H), 7.73-7.65 (m, 1H), 7.39-7.22 (m, 1H).

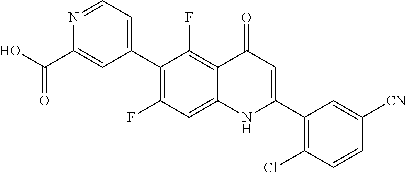 [SMILES: N#Cc1ccc(Cl)c(-c2cc(=O)c3c(F)c(-c4ccnc(C(=O)O)c4)c(F)cc3[nH]2)c1]

4-(2-(2-chloro-5-cyanophenyl)-5,7-difluoro-4-oxo-1,4-dihydroquinolin-6-yl)picolinic acid: This material was prepared using the same synthetic sequence that was described for 5-(2-(2-chloro-5-cyanophenyl)-5,7-difluoro-4-oxo-1,4-dihydroquinolin-6-yl)-2,3-difluorobenzoic acid. ¹H NMR (400 MHz, DMSO-d₆) δ 11.3 (s, 1H), 8.86 (d, J=4.8 Hz, 1H), 8.25 (d, J=2.0 Hz, 1H), 8.19 (s, 1H), 8.09 (dd, J=2.0, 8.4 Hz, 1H), 7.93 (d, J=8.4 Hz, 1H), 7.82 (br d, J=4.8 Hz, 1H), 7.41 (br d, J=10.8 Hz, 1H), 6.24-6.17 (m, 1H).

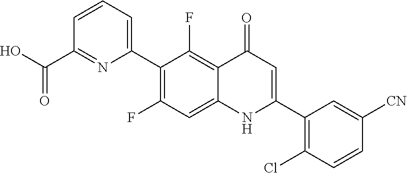 [SMILES: N#Cc1ccc(Cl)c(-c2cc(=O)c3c(F)c(-c4cccc(C(=O)O)n4)c(F)cc3[nH]2)c1]

6-(2-(2-chloro-5-cyanophenyl)-5,7-difluoro-4-oxo-1,4-dihydroquinolin-6-yl)picolinic acid: This material was prepared using the same synthetic sequence that was described for 5-(2-(2-chloro-5-cyanophenyl)-5,7-difluoro-4-oxo-1,4-dihydroquinolin-6-yl)-2,3-difluorobenzoic acid. ¹H NMR (400 MHz, DMSO-d₆) δ 11.3 (s, 1H), 8.86 (d, J=4.8 Hz, 1H), 8.25 (d, J=2.0 Hz, 1H), 8.19 (s, 1H), 8.09 (dd, J=2.0, 8.4 Hz, 1H), 7.93 (d, J=8.4 Hz, 1H), 7.82 (br d, J=4.8 Hz, 1H), 7.41 (br d, J=10.8 Hz, 1H), 6.24-6.17 (m, 1H).

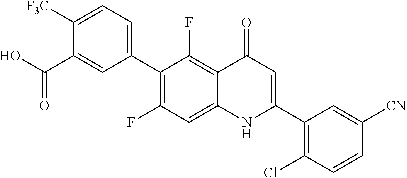 [SMILES: N#Cc1ccc(Cl)c(-c2cc(=O)c3c(F)c(-c4ccc(C(F)(F)F)c(C(=O)O)c4)c(F)cc3[nH]2)c1]

5-(2-(2-chloro-5-cyanophenyl)-5,7-difluoro-4-oxo-1,4-dihydroquinolin-6-yl)-2-(trifluoromethyl)benzoic acid: This material was prepared using the same synthetic sequence that was described for 5-(2-(2-chloro-5-cyanophenyl)-5,7-difluoro-4-oxo-1,4-dihydroquinolin-6-yl)-2,3-difluorobenzoic acid. ¹H NMR (400 MHz, DMSO-d₆) δ 14.41-13.33 (m, 1H), 12.60-11.96 (m, 1H), 8.27 (s, 1H), 8.10 (d, J=8.4 Hz, 1H), 8.00 (d, J=8.3 Hz, 1H), 7.97-7.91 (m, 2H), 7.90-7.83 (m, 1H), 7.28 (d, J=9.9 Hz, 1H), 6.13 (br s, 1H).

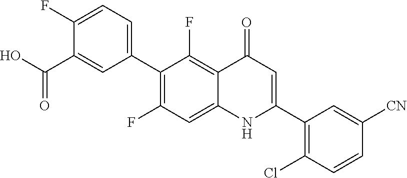 [SMILES: N#Cc1ccc(Cl)c(-c2cc(=O)c3c(F)c(-c4ccc(F)c(C(=O)O)c4)c(F)cc3[nH]2)c1]

### 5-(2-(2-chloro-5-cyanophenyl)-5,7-difluoro-4-oxo-1,4-dihydroquinolin-6-yl)-2-fluorobenzoic acid

### Step 1, methyl 3′-acetyl-4′-amino-2′,4,6′-trifluoro-[1,1′-biphenyl]-3-carboxylate

To a mixture of (4-fluoro-3-(methoxycarbonyl)phenyl)boronic acid (600 mg, 3.0 mmol, 1.5 equiv.) and 1-(6-amino-2,4-difluoro-3-iodophenyl)ethan-1-one (600 mg, 2.0 mmol, 1.0 equiv.) in dioxane (4.5 mL) and water (1.5 mL) was added Pd(dppf)Cl₂—CH₂Cl₂ (165 mg, 202 mol, 0.1 equiv.) and K₂CO₃ (838 mg, 6.1 mmol, 3.0 equiv.) at 20° C. under N₂. The mixture was stirred at 80° C. for 12 hours. The reaction mixture was poured into water (20 mL) and the aqueous layer was extracted with ethyl acetate (3×40 mL). The combined organic layers were washed with brine (2×40 mL), dried over Na₂SO₄, filtered, and concentrated under reduced pressure. The residue was purified by silica gel column chromatography (10:1 to 2:1 petroleum ether:ethyl acetate) to afford the title compound (560 mg, 83% yield) as a brown solid. ¹H NMR (400 MHz, METHANOL-d₄) δ 7.92 (br d, J=6.8 Hz, 1H), 7.68-7.56 (m, 1H), 7.29 (dd, J=8.6, 10.6 Hz, 1H), 6.43 (dd, J=1.6, 12.4 Hz, 1H), 3.98-3.87 (m, 3H), 2.55 (d, J=8.8 Hz, 3H).

### Step 2, methyl 3′-acetyl-4′-(2-chloro-5-cyanobenzamido)-2′,4,6′-trifluoro-[1,1′-biphenyl]-3-carboxylate

To a mixture of methyl 3′-acetyl-4′-amino-2′,4,6′-trifluoro-[1,1′-biphenyl]-3-carboxylate (490 mg, 1.5 mmol, 1 equiv.) in THF (5 mL) was added NaH (66.6 mg, 1.7 mmol, 1.1 equiv.; 60% dispersion in oil) and 2-chloro-5-cyanobenzoyl chloride (364 mg, 1.8 mmol, 1.2 equiv.) at 0° C. under N₂. The mixture was stirred at 20° C. for 12 hours. The mixture was added to a saturated aqueous solution of NH₄Cl (15 mL) and the mixture was extracted with ethyl acetate (3×30 mL). The combined organic layers were washed with brine (2×30 mL), dried over Na₂SO₄, filtered, and concentrated under reduced pressure. The crude product was washed with ethyl acetate (2×3 mL), filtered, and concentrated under reduced pressure to afford the title compound (540 mg, 71% yield) as a white solid. ¹H NMR (400 MHz, DMSO-d₆) δ 11.20 (s, 1H), 8.12 (s, 1H), 8.08-8.00 (m, 2H), 7.85 (br d, J=8.4 Hz, 2H), 7.67-7.45 (m, 2H), 3.88 (s, 3H), 2.58 (br d, J=3.6 Hz, 3H).

### Step 3, methyl 5-(2-(2-chloro-5-cyanophenyl)-5,7-difluoro-4-oxo-1,4-dihydroquinolin-6-yl)-2-fluorobenzoate

To a mixture of methyl 3′-acetyl-4′-(2-chloro-5-cyanobenzamido)-2′,4,6′-trifluoro-[1,1′-biphenyl]-3-carboxylate (540 mg, 1.1 mmol, 1 equiv.) in dioxane (2 mL) was added NaOH (44.3 mg, 1.1 mmol, 1 equiv.) at 20° C. under N₂. The mixture was stirred at 110° C. for 2 hours. The pH of the residue was adjusted to 3-4 with HCl (1M). Then the mixture was diluted with water (10 mL), the mixture was stirred at 20° C. for 10 minutes. The mixture was filtered and the filter cake was concentrated under reduced pressure to give a crude product. The crude product was washed with acetonitrile (2×2 mL), the mixture was filtered the filter cake was concentrated under reduced pressure to afford the title compound (400 mg, 77% yield) as a white solid. ¹H NMR (400 MHz, DMSO-d₆) δ 12.31-12.15 (m, 1H), 8.29-8.24 (m, 1H), 8.12 (s, 1H), 8.03-7.91 (m, 3H), 7.87-7.75 (m, 1H), 7.58-7.45 (m, 1H), 7.33-7.22 (m, 1H), 6.11 (br s, 1H), 3.89 (s, 3H).

### Step 4, 5-(2-(2-chloro-5-cyanophenyl)-5,7-difluoro-4-oxo-1,4-dihydroquinolin-6-yl)-2-fluorobenzoic acid

To a mixture of methyl 5-(2-(2-chloro-5-cyanophenyl)-5,7-difluoro-4-oxo-1,4-dihydroquinolin-6-yl)-2-fluorobenzoate (200 mg, 427 μmol, 1 equiv.) in THF (2.1 mL) and water (0.9 mL) was added LiOH·H₂O (35.8 mg, 853 μmol, 2 equiv.) at 20° C. under N₂. The mixture was stirred at 20° C. for 6 hours. The reaction mixture was poured into water (10 mL) and the pH was adjusted to 7 with HCl (1 M). A solid precipitated from the mixture which was washed with water (2 mL), then the mixture was filtered and the filter cake was dried under vacuum to afford the title compound (180 mg, 89% yield, 96% purity) as a white solid. ¹H NMR (400 MHz, DMSO-d₆) δ 13.65-13.22 (m, 1H), 12.34-11.99 (m, 1H), 8.26 (d, J=1.8 Hz, 1H), 8.10 (dd, J=2.0, 8.4 Hz, 1H), 8.02-7.90 (m, 2H), 7.78 (br d, J=3.6 Hz, 1H), 7.49 (dd, J=8.6, 10.6 Hz, 1H), 7.27 (br d, J=10.0 Hz, 1H), 6.35-5.94 (m, 1H).

### Example 1

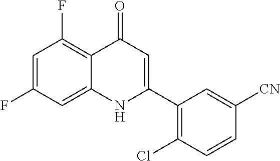 [SMILES: N#Cc1ccc(Cl)c(-c2cc(=O)c3c(F)cc(F)cc3[nH]2)c1]

### 4-chloro-3-(5,7-difluoro-4-oxo-1,4-dihydroquinolin-2-yl)benzonitrile

### Scheme 1. 2-chloro-5-cyanobenzoyl Chloride

A solution of 2-chloro-5-cyano-benzoic acid (2.5 g, 13.8 mmol) in SOCl₂ (25 mL) was stirred at 80° C. for 1 hour. The reaction mixture was then cooled to room temperature and concentrated under reduced pressure to afford the title compound (2.8 g, crude) as a yellow solid, the product was used directly in next step.

### Scheme 1, step 1. 1-(2-amino-4,6-difluorophenyl)ethanone

To a solution of 3,5-difluoroaniline (8.9 g, 68.9 mmol, 1.0 equiv.) in CH₃CN (85 mL) was added BCl₃ (1 M, 72.4 mL, 1.05 equiv.) at 0° C. Then AlCl₃ (10.1 g, 75.8 mmol, 4.1 mL, 1.1 equiv.) was added to the mixture in three portions and the mixture was then stirred at 80° C. for 16 hours. The mixture was cooled to 0° C. and then aqueous HCl (4M, 80 mL) was added and the mixture was stirred at 80° C. for 2 hours. The mixture was cooled to room temperature and extracted with EtOAc (2×150 mL). The combined organic layers were washed with saturated aqueous NaHCO₃ solution (2×50 mL), dried over Na₂SO₄, filtered, and concentrated under reduced pressure to afford the title compound (8.0 g, 68% yield) as a light-yellow solid. LCMS: calculated for [M+H]⁺ (C₈H₇F₂NO) requires m/z=172.0, found m/z=172.1. ¹H NMR (400 MHz, CDCl₃), δ 6.5 (br s, 2H), 6.0-6.2 (m, 2H), 2.6 (d, J=8.4 Hz, 3H).

### Scheme 1, step 2. N-(2-acetyl-3,5-difluorophenyl)-2-chloro-5-cyanobenzamide

To a solution of 1-(2-amino-4,6-difluoro-phenyl)ethanone (2 g, 11.7 mmol, 1.0 equiv.) in THF (20 mL) was added NaH (467 mg, 11.7 mmol, 60% dispersion in oil, 1.0 equiv.) at 0° C. The mixture was stirred for 30 minutes before the dropwise addition of a solution of 2-chloro-5-cyano-benzoyl chloride (2.6 g, 12.8 mmol, 1.1 equiv.) in THF (10 mL). The mixture was stirred at room temperature for 16 hours. The reaction mixture was quenched by the addition saturated aqueous NH₄Cl (15 mL) at 15° C., diluted with water (20 mL), and filtered. The filter cake was triturated with EtOAc (20 mL) and filtered to afford the title compound (2.4 g, 61% yield) as a white solid. LCMS: calculated for [M+H]+(C₁₆H₉F₃N₂O₂) requires m/z=335.0, found m/z=335.0. ¹H NMR (400 MHz, DMSO-d₆) δ 11.2 (s, 1H), 8.1 (d, J=2.0 Hz, 1H), 8.0 (dd, J=8.4, 2.2 Hz, 1H), 7.8 (d, J=8.4 Hz, 1H), 7.5-7.5 (m, 1H), 7.3 (ddd, J=11.2, 8.8, 2.2 Hz, 1H), 2.5-2.6 (m, 3H).

### Scheme 1, Step 3. 4-chloro-3-(5,7-difluoro-4-oxo-1,4-dihydroquinolin-2-yl)benzonitrile

To a solution of N-(2-acetyl-3,5-difluoro-phenyl)-2-chloro-5-cyano-benzamide (2.5 g, 7.5 mmol, 1.0 equiv.) in dioxane (40 mL) was added NaOH (3.0 g, 74.7 mmol, 10.0 equiv.). The mixture was stirred at 110° C. for 1.5 hours. The pH of the reaction mixture was adjusted to 5 with aqueous HCl (1 M) and then diluted with water (30 mL) and extracted with EtOAc (3×100 mL). The combined organic layers were washed with brine (2×100 mL), dried over Na₂SO₄, filtered, and concentrated under reduced pressure to afford a crude residue that was purified by preparative HPLC (column: Welch Xtimate C18 250×70 mm×10 um; mobile phase: 15-45% acetonitrile in water (10 mM NH₄HCO₃)). This afforded the title compound (570 mg, 24% yield, 98% purity) as a white solid after concentration under reduced pressure. LCMS: calculated for [M+H]⁺ (C₁₆H₇ClF₂N₂O) requires m/z=317.0, found m/z=317.0. ¹H NMR (400 MHz, DMSO-d₆) δ 9.2-10.3 (m, 1H), 8.2 (d, J=2.0 Hz, 1H), 8.0 (dd, J=8.4, 2.0 Hz, 1H), 7.9 (d, J=8.4 Hz, 1H), 7.0-7.2 (m, 2H), 6.1 (s, 1H).

### Example 2

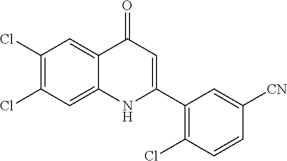 [SMILES: N#Cc1ccc(Cl)c(-c2cc(=O)c3cc(Cl)c(Cl)cc3[nH]2)c1]

### 4-chloro-3-(6,7-dichloro-4-oxo-1,4-dihydroquinolin-2-yl)benzonitrile

### Scheme 1, step 1. 1-(2-amino-4,5-dichlorophenyl)ethenone

To a solution of 3,4-dichloroaniline (2.0 g, 12.4 mmol, 1.0 equiv.) in ACN (20 mL) was added BCl₃ (1 M, 13.0 mL, 1.05 eq) at 0° C. Then AlCl₃ (1.81 g, 13.58 mmol, 742 μL, 1.1 equiv.) was added in three portions and the mixture was stirred at 80° C. for 16 hours. The mixture was cooled to 0° C., and aqueous HCl (4 M, 5 mL) was added and the mixture was stirred at 80° C. for 4 hours. The reaction was cooled to room temperature, extracted with ethyl acetate, dried over Na₂SO₄, and concentrated under reduced pressure. The residue was purified by column chromatography (SiO₂, 30:1 petroleum ether: EtOAc) to afford the title compound (170 mg, 6.7% yield) as a white solid. LCMS: calculated for [M+H]+(C₈H₇Cl₂NO) requires m/z=204.1, found m/z=204.1. ¹H NMR (400 MHz, DMSO-d₆) δ 7.92 (s, 1H), 7.39 (br s, 2H), 7.02 (s, 1H), 2.52 (s, 3H).

### Scheme 1, step 2. N-(2-acetyl-4,5-dichlorophenyl)-2-chloro-5-cyanobenzamide

To a solution of 1-(2-amino-4,5-dichlorophenyl)ethanone (125 mg, 614 μmol, 1.0 equiv.) in DCM (1 mL) was added TEA (171 uL, 1.2 mmol, 2.0 equiv.) at room temperature under an atmosphere of nitrogen. The mixture was stirred at room temperature for 15 minutes before 2-chloro-5-cyano-benzoyl chloride (172 mg, 860 umol, 1.4 equiv.) was added dropwise as a solution in DCM (1 mL). The reaction mixture was stirred at room temperature for 5 hours. The mixture was poured into water (0.5 mL) and stirred for 1 minute. The aqueous phase was extracted with DCM (3×3 mL). The combined organic layers were dried over anhydrous Na₂SO₄, filtered and concentrated under reduced pressure. The crude product was triturated with MeOH to afford the title compound (100 mg, crude) as a yellow solid. LCMS: calculated for [M+H]⁺ (C₁₆H₉Cl₃N₂O₂) requires m/z=366.9, found m/z=366.9.

### Scheme 1, Step 3. 4-chloro-3-(6,7-dichloro-4-oxo-1,4-dihydroquinolin-2-yl)benzonitrile

To a solution of N-(2-acetyl-4,5-dichlorophenyl)-2-chloro-5-cyanobenzamide (90 mg, 244 μmol, 1.0 equiv.) in dioxane (2.0 mL) was added NaOH (98 mg, 2.5 mmol, 10 equiv.). The mixture was stirred at 110° C. for 1.5 hours. The pH of the reaction mixture was adjusted to 5 by the addition of aqueous HCl (1 M). The mixture was then diluted with water (2 mL) and extracted with EtOAc (4×5 mL). The combined organic layers were dried over Na₂SO₄, filtered, and concentrated under reduced pressure to give a residue that was first purified by preparative TLC (SiO₂, 10:1 DCM:MeOH) and then by preparative HPLC (column: Waters Xbridge BEH C18 100×30 mm×10 um; mobile phase: 35-65% ACN in water (10 mM NH₄HCO₃)) to afford the title compound (3.8 mg, 4.4% yield, 99% purity) as a white solid. LCMS: calculated for [M+H]⁺ (C₁₆H₇Cl₃N₂O) requires m/z=348.0, found m/z=348.0. ¹H NMR (400 MHz, MeOH-d₄) δ 8.38 (s, 1H), 8.04 (d, J=2.0 Hz, 1H), 7.92 (dd, J=1.6, 8.4 Hz, 1H), 7.85-7.80 (m, 2H), 6.39 (s, 1H).

The compounds in Table 1 were prepared following Scheme 1 using similar procedures to those described for Examples 1 and 2.

TABLE 1

| | | | | LCMS |
|---|---|---|---|---|
| Example | | | | Exact |
| number | Structure | IUPAC Name | ¹H NMR | Mass |
| 3 | 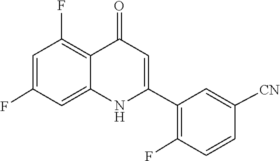 [SMILES: N#Cc1ccc(F)c(-c2cc(=O)c3c(F)cc(F)cc3[nH]2)c1] | 3-(5,7-difluoro-4- oxo-1,4- dihydroquinolin- 2-yl)-4- fluorobenzonitrile | (400 MHz, DMSO-d₆) δ 12.1 (br s, 1H), 8.3 (br d, J = 5.4 Hz, 1H), 8.2 (ddd, J = 8.6, 4.6, 2.0 Hz, 1H), 7.7 (t, J = 9.4 Hz, 1H), 7.1- 7.3 (m, 2H), 6.1- 6.4 (m, 1H). | Calculated m/z 319.0, found m/z 319.0 |
| 4 | 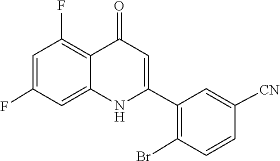 [SMILES: N#Cc1ccc(Br)c(-c2cc(=O)c3c(F)cc(F)cc3[nH]2)c1] | 4-bromo-3-(5,7- difluoro-4-oxo- 1,4- dihydroquinolin- 2-yl)benzonitrile | (400 MHz, DMSO-d₆) δ 8.08 (s, 1H), 8.01 (d, J = 8.4 Hz, 1H), 7.90 (s, 1H), 7.08 (br d, J = 10.2 Hz, 1H), 6.94- 7.04 (m, 1H), 6.01 (s, 1H) | Calculated m/z 361.0, found m/z 361.0 |
| 5 | 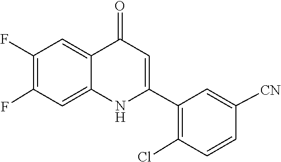 [SMILES: N#Cc1ccc(Cl)c(-c2cc(=O)c3cc(F)c(F)cc3[nH]2)c1] | 4-chloro-3-(6,7- difluoro-4-oxo- 1H-quinolin-2- yl)benzonitrile | (400 MHz, DMSO-d₆) δ 12.22 (br s, 1H), 8.25 (br s, 1H), 8.09 (br d, J = 8.8 Hz, 1H), 8.05- 7.97 (m, 1H), 7.93 (br d, J = 8.4 Hz, 1H), 7.54 (br s, 1H), 6.16 (br s, 1H) | Calculated m/z 317.0, found m/z 317.0 |
| 6 | 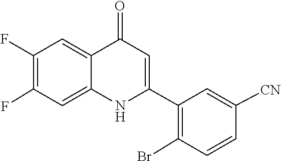 [SMILES: N#Cc1ccc(Br)c(-c2cc(=O)c3cc(F)c(F)cc3[nH]2)c1] | 4-bromo-3-(6,7- difluoro-4-oxo- 1,4- dihydroquinolin- 2-yl)benzonitrile | (400 MHz, DMSO-d₆) δ = 12.2-12.3 (m, 1H) 8.2 (br s, 1H) 8.1 (d, J = 8.4 Hz, 1H) 7.9-8.1 (m, 2H) 7.5-7.6 (m, 1H) 6.1 (br d, J = 5.4 Hz, 1H) | Calculated m/z 361.0, found m/z 361.0 |
| 7 | 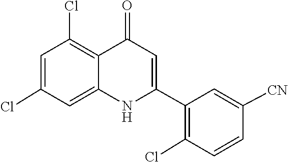 [SMILES: N#Cc1ccc(Cl)c(-c2cc(=O)c3c(Cl)cc(Cl)cc3[nH]2)c1] | 4-chloro-3-(5,7- dichloro-4-oxo- 1,4- dihydroquinolin- 2-yl)benzonitrile | (400 MHz, DMSO-d₆) δ 12.08 (s, 1H), 8.25 (d, J = 1.4 Hz, 1H), 8.09 (dd, J = 1.8, 8.4 Hz, 1H), 7.93 (d, J = 8.6 Hz, 1H), 7.53 (s, 1H), 7.45 (s, 1H), 6.13 (s, 1H) | Calculated m/z 349.0, found m/z 348.9 |
| 8 | 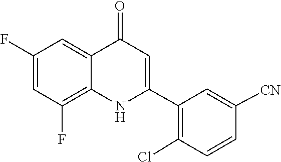 [SMILES: N#Cc1ccc(Cl)c(-c2cc(=O)c3cc(F)cc(F)c3[nH]2)c1] | 4-chloro-3-(6,8- difluoro-4-oxo- 1,4- dihydroquinolin- 2-yl)benzonitrile | (400 MHz, MeOD-d4) δ 8.04 (d, J = 2.0 Hz, 1H), 7.91 (dd, J = 2.0, 8.4 Hz, 1H), 7.82 (m, 1H), 7.79-7.74 (m, 1H), 7.54 (m, 1H), 6.41 (s, 1H) | Calculated m/z 317.0, found m/z 316.9 |
| 13 | 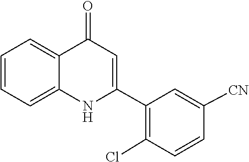 [SMILES: N#Cc1ccc(Cl)c(-c2cc(=O)c3ccccc3[nH]2)c1] | 4-chloro-3-(4- oxo-1,4- dihydroquinolin- 2-yl)benzonitrile | (400 MHz, DMSO-d6) δ 12.10-11.95 (br s, 1H), 8.25 (d, J = 1.8 Hz, 1H), 8.16-8.10 (m, 1H), 8.08 (dd, J = 2.0, 8.4 Hz, 1H), 7.92 (d, J = 8.4 Hz, 1H), 7.74- | Calculated m/z 281.0, found m/z 281.0 |
| | | | 7.67 (m, 1H), | |
| | | | 7.59 (br d, J = 8.2 | |
| | | | Hz, 1H), 7.38 (t, | |
| | | | J = 7.6 Hz, 1H), | |
| | | | 6.10 (br s, 1H) | |
| 14 | 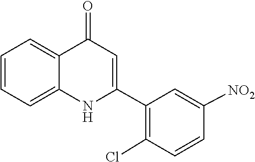 [SMILES: O=c1cc(-c2cc([N+](=O)[O-])ccc2Cl)[nH]c2ccccc12] | 2-(2-chloro-5- nitrophenyl) quinolin-4(1H)-one | (400 MHz, DMSO-d6) δ 8.51 (br d, J = 1.4 Hz, 1H), 8.42 (br d, J = 8.6 Hz, 1H), 8.14 (br d, J = 8.0 Hz, 1H), 8.00 (d, J = 8.8 Hz, 1H), 7.70 (br d, J = 7.0 Hz, | Calculated m/z 301.0, found m/z 301.0 |
| | | | 1H), 7.60 (s, 1H), | |
| | | | 7.38 (br t, J = 7.6 | |
| | | | Hz, 1H), 6.13 (s, | |
| | | | 1H) | |
| 15 | 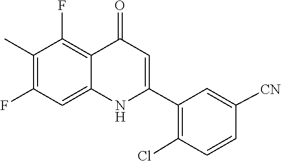 [SMILES: Cc1c(F)cc2[nH]c(-c3cc(C#N)ccc3Cl)cc(=O)c2c1F] | 4-chloro-3-(5,7- difluoro-6- methyl-4-oxo-1,4- dihydroquinolin- 2-yl)benzonitrile | (400 MHz, DMSO-d₆) δ 12.01 (br s, 1H), 8.22 (s, 1H), 8.07 (dd, J = 1.6, 8.4 Hz, 1H), 7.91 (d, J = 8.4 Hz, 1H), 7.11 (br d, J = 7.6 Hz, 1H), 6.03 (br s, 1H), 2.19 (br s, 3H) | Calculated m/z 331.0, found m/z 331.0 |
| 16 | 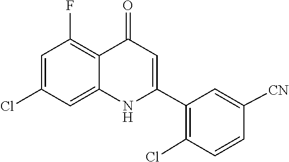 [SMILES: N#Cc1ccc(Cl)c(-c2cc(=O)c3c(F)cc(Cl)cc3[nH]2)c1] | 4-chloro-3-(7- chloro-5-fluoro-4- oxo-1,4- dihydroquinolin- 2-yl)benzonitrile | (400 MHz, DMSO-d₆) δ 8.24 (d, J = 2.2 Hz, 1H), 8.09 (dd, J = 2.2, 8.4 Hz, 1H), 7.93 (d, J = 8.4 Hz, 1H), 7.47 (s, 1H), 7.29 (dd, J = 1.8, 11.4 Hz, 1H), 6.18 (br s, 1H) | Calculated m/z 333.0, found m/z 333.0 |
| 17 | 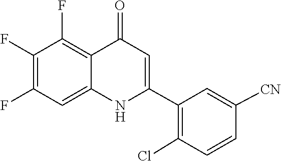 [SMILES: N#Cc1ccc(Cl)c(-c2cc(=O)c3c(F)c(F)c(F)cc3[nH]2)c1] | 4-chloro-3-(5,6,7- trifluoro-4-oxo- 1,4- dihydroquinolin- 2-yl)benzonitrile | (400 MHz, DMSO-d₆) δ 12.2 (s, 1H) 8.2 (d, J = 1.0 Hz, 1H) 8.1 (br d, J = 8.3 Hz, 1H) 7.9 (d, J = 8.4 Hz, 1H) 7.3 (br dd, J = 9.7, 6.8 Hz, 1H) 6.1 (s, 1H) | Calculated m/z 335.0, found m/z 334.9 |
| 18 | 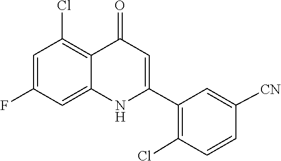 [SMILES: N#Cc1ccc(Cl)c(-c2cc(=O)c3c(Cl)cc(F)cc3[nH]2)c1] | 4-chloro-3-(5- chloro-7-fluoro-4- oxo-1,4- dihydroquinolin- 2-yl)benzonitrile | (400 MHz, DMSO-d₆) δ 12.07 (s, 1H), 8.24 (d, J = 1.8 Hz, 1H), 8.09 (dd, J = 1.8, 8.4 Hz, 1H), 7.93 (d, J = 8.4 Hz, 1H), 7.35 (dd, J = 2.4, 8.9 Hz, 1H), 7.26 (dd, J = 2.2, 9.6 | Calculated m/z 333.0, found m/z 333.0 |
| | | | Hz, 1H), 6.10 (s, | |
| | | | 1H). | |
| 19 | 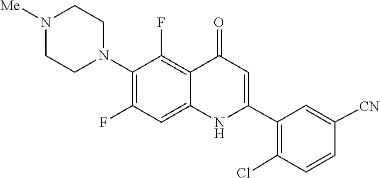 [SMILES: CN1CCN(c2c(F)cc3[nH]c(-c4cc(C#N)ccc4Cl)cc(=O)c3c2F)CC1] | 4-chloro-3-(5,7- difluoro-6-(4- methylpiperazin- 1-yl)-4-oxo-1,4- dihydroquinolin- 2-yl)benzonitrile | (400 MHz, DMSO-d₆) δ 10.67-10.57 (m, 1H), 8.21 (d, J = 2.0 Hz, 1H), 8.11-8.06 (m, 1H), 7.92 (d, J = 8.4 Hz, 1H), 7.26 (br d, J = 11.6 Hz, 1H), 6.15 (br d, J = 1.4 Hz, 1H), 3.49 (br t, | Calculated m/z 415.1, found m/z 415.0 |
| | | | J = 13.6 Hz, 4H), | |
| | | | 3.32 (br d, J = | |
| | | | 13.2 Hz, 2H), | |
| | | | 3.24-3.13 (m, | |
| | | | 2H), 2.85 (d, J = | |
| | | | 4.6 Hz, 3H) | |
| 20 | 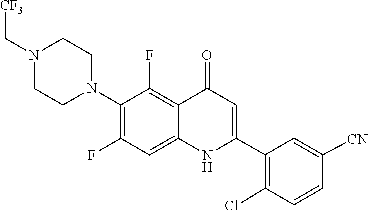 [SMILES: N#Cc1ccc(Cl)c(-c2cc(=O)c3c(F)c(N4CCN(CC(F)(F)F)CC4)c(F)cc3[nH]2)c1] | 4-chloro-3-(5,7- difluoro-4-oxo-6- (4-(2,2,2- trifluoroethyl) piperazin-1- yl)-1,4- dihydroquinolin- 2-yl)benzonitrile | (400 MHz, DMSO-d₆) δ 11.98 (br s, 1H), 8.20 (br s, 1H), 8.07 (br d, J = 7.8 Hz, 1H), 7.91 (d, J = 8.4 Hz, 1H), 7.19-7.01 (m, 1H), 6.00 (br s, 1H), 3.39-3.19 (m, 6H), 2.76 (br s, 4H) | Calculated m/z 483.1, found m/z 482.9 |
| 21 | 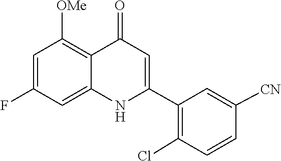 [SMILES: COc1cc(F)cc2[nH]c(-c3cc(C#N)ccc3Cl)cc(=O)c12] | 4-chloro-3-(7- fluoro-5-methoxy- 4-oxo-1,4- dihydroquinolin- 2-yl)benzonitrile | (400 MHz, DMSO-d₆) δ 11.70 (br s, 1H), 8.37-7.76 (m, 3H), 6.87-6.59 (m, 2H), 5.93 (br s, 1H), 3.84 (br s, 3H) | Calculated m/z 329.0, found m/z 328.9 |
| 22 | 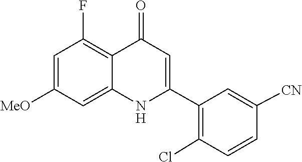 [SMILES: COc1cc(F)c2c(=O)cc(-c3cc(C#N)ccc3Cl)[nH]c2c1] | 4-chloro-3-(5- fluoro-7-methoxy- 4-oxo-1,4- dihydroquinolin- 2-yl)benzonitrile | (400 MHz, DMSO-d₆) δ 8.23 (d, J = 2.2 Hz, 1H), 8.07 (d, J = 2.0 Hz, 1H), 7.93 (d, J = 8.4 Hz, 1H), 6.88-6.77 (m, 2H), 6.08 (s, 1H), 3.86 (s, 3H) | Calculated m/z 329.0, found m/z 328.9 |

### Example 9

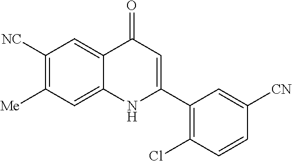 [SMILES: Cc1cc2[nH]c(-c3cc(C#N)ccc3Cl)cc(=O)c2cc1C#N]

### 2-(2-chloro-5-cyanophenyl)-7-methyl-4-oxo-1,4-dihydroquinoline-6-carbonitrile

### Scheme 2, step 1. 4-amino-5-iodo-2-methyl-benzonitrile

To a solution of 4-amino-2-methyl-benzonitrile (400 mg, 3.0 mmol, 1.0 equiv.) in AcOH (5 mL) was added NIS (681 mg, 3.0 mmol, 1.0 equiv.). Then the mixture was stirred at room temperature for 3 hours. The mixture was quenched with H₂O (100 mL) and extracted with ethyl acetate (2×30 mL). The combined organic layers were washed with saturated aqueous NaHCO₃ (10 mL) and brine (5 mL) and then dried with anhydrous Na₂SO₄, filtered and concentrated under reduced pressure. The residue was purified by silica gel chromatography (3:1 petroleum ether:EtOAc) to afford the title compound (580 mg, crude) as a brown solid. LCMS: calculated for [M+H]⁺ (C₈H₇IN₂) requires m/z=259.0, found m/z=259.0. ¹H NMR (400 MHz, CDCl₃-d) δ 7.92-7.73 (m, 1H), 6.59 (s, 1H), 4.54 (br d, J=1.3 Hz, 2H), 2.40 (s, 3H).

### Scheme 2, step 2. 5-acetyl-4-amino-2-methyl-benzonitrile

To a solution of 4-amino-5-iodo-2-methyl-benzonitrile (450 mg, 1.7 mmol, 1.0 equiv.) and tributyl(1-ethoxyvinyl)stannane (756 mg, 2.1 mmol, 1.2 equiv.) in toluene (12 mL) was added Pd(PPh₃)₄ (101 mg, 87.2 μmol, 0.05 equiv.) at room temperature. The mixture was stirred at 120° C. for 16 hours under an atmosphere of nitrogen. The mixture was cooled to room temperature and quenched by the addition of aqueous HCl (1 N, 2 mL) and stirred at room temperature for 30 minutes. Then the mixture was added to an aqueous solution of KF (20 mL) and stirred at room temperature for 1 hour. Then the mixture was diluted with water (40 mL) and extracted with EtOAc (3×30 mL). The combined organic layers were washed with brine (5 mL), dried over sodium sulfate, filtered and concentrated under reduced pressure. The residue was purified by silica gel chromatography (10:1 to 3:1 petroleum ether:EtOAc) to afford the title compound (160 mg, 53% yield) as a white solid. LCMS: calculated for [M+H]⁺ (C₁₀H₁₀N₂O) requires m/z=175.1, found m/z=175.1. ¹H NMR (400 MHz, CDCl₃-d) δ 8.12-7.90 (m, 1H), 6.53 (s, 1H), 2.59 (s, 3H), 2.46 (s, 3H).

### Scheme 2, step 3. N-(2-acetyl-4-cyano-5-methyl-phenyl)-2-chloro-5-cyano-benzamide

To a solution of 5-acetyl-4-amino-2-methyl-benzonitrile (120 mg, 689 μmol, 1.0 equiv.) in THF (5 mL) was added NaH (33 mg, 827 μmol, 60% dispersion in oil, 1.2 equiv.). After the mixture was stirred at 0° C. for 10 minutes, 2-chloro-5-cyano-benzoyl chloride (165 mg, 825 μmol, 1.2 equiv.) was added and the mixture was stirred at room temperature for 16 hours under an atmosphere of nitrogen. The mixture was poured into a saturated aqueous solution of NH₄Cl (5 mL) slowly and stirred for 5 minutes. The aqueous phase was extracted with EtOAc (2×30 mL). The combined organic layers were washed with brine (5 mL), dried over anhydrous Na₂SO₄, filtered and concentrated. The residue was purified by preparative TLC (2:1 petroleum ether:EtOAc) to afford the title compound (12 mg, 35 μmol, 5.2% yield) as a white solid. LCMS: calculated for [M+H]⁺ (C₁₈H₁₂ClN₃O₂) requires m/z=338.1, found m/z=338.0. ¹H NMR (400 MHz, DMSO-d₆) δ 11.96 (br s, 1H), 8.54-8.49 (m, 1H), 8.46 (s, 1H), 8.25 (br s, 1H), 8.12-8.05 (m, 1H), 7.89 (br d, J=8.2 Hz, 1H), 2.68 (br s, 3H), 2.59 (br s, 3H).

### Scheme 2, step 4. 2-(2-chloro-5-cyano-phenyl)-7-methyl-4-oxo-1H-quinoline-6-carbonitrile

To a solution of N-(2-acetyl-4-cyano-5-methyl-phenyl)-2-chloro-5-cyano-benzamide (10 mg, 29.6 μmol, 1.0 equiv.) in dioxane (1.0 mL) was added NaOH (11.8 mg, 296 μmol, 10 equiv.). The reaction was stirred at 110° C. for 2 hours under an atmosphere of nitrogen. The pH of the mixture was adjusted to 5-6 with aqueous 1N HCl. The aqueous phase was extracted with EtOAc (2×30 mL). The combined organic layers were washed with brine (5 mL), dried over anhydrous Na₂SO₄, filtered and concentrated. The residue was purified by preparative HPLC (Column: Phenomenex Luna C18 75×30 mm×3 um; mobile phase: 15-45% ACN in water (0.2% FA)) to afford the title compound (2.3 mg, 24% yield, 98% purity) as a white solid. LCMS: calculated for [M+H]⁺ (C₁₈H₁₀ClN₃O) requires m/z=320.1, found m/z=320.0. ¹H NMR (400 MHz, DMSO-d₆) δ 8.46-8.38 (m, 1H), 8.24 (d, J=1.9 Hz, 1H), 8.08 (dd, J=2.0, 8.4 Hz, 1H), 7.92 (d, J=8.4 Hz, 1H), 7.53 (s, 1H), 6.21 (s, 1H), 2.58 (s, 3H).

The compounds in Table 2 were prepared following Scheme 2 using similar procedures to those described for Example 9.

TABLE 2

| | | | | LCMS |
|---|---|---|---|---|
| Example | | | | Exact |
| number | Structure | IUPAC Name | ¹H NMR | Mass |
| 10 | 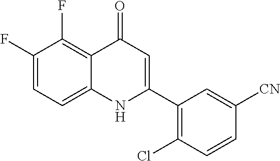 [SMILES: N#Cc1ccc(Cl)c(-c2cc(=O)c3c(F)c(F)ccc3[nH]2)c1] | 4-chloro-3-(5,6- difluoro-4-oxo- 1,4- dihydroquinolin- 2-yl)benzonitrile | (400 MHz, DMSO-d₆) δ 12.30-12.12 (m, 1H), 8.26-8.18 (m, 1H), 8.06 (dd, J = 2.0, 8.4 Hz, 1H), 7.91 (d, J = 8.4 Hz, 1H), 7.84- 7.71 (m, 1H), 7.50-7.35 (m, 1H), 6.20-6.06 (m, 1H). | Calculated m/z 317.0, found m/z 316.9 |
| 11 | 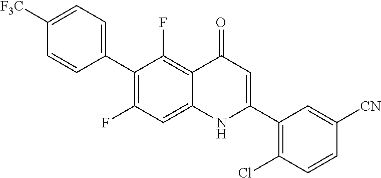 [SMILES: N#Cc1ccc(Cl)c(-c2cc(=O)c3c(F)c(-c4ccc(C(F)(F)F)cc4)c(F)cc3[nH]2)c1] | 4-chloro-3-(5,7- difluoro-4-oxo-6- (4- (trifluoromethyl) phenyl)-1,4- dihydroquinolin- 2-yl)benzonitrile | (400 MHz, MeOD-d₄) δ 8.13 (d, J = 12.0 Hz, 1H), 7.99 (dd, J = 2.0, 8.4 Hz, 1H), 7.90-7.82 (m, 3H), 7.74 (d, J = 8.0 Hz, 2H), 7.38 (dd, J = 1.6, 10.2 Hz, 1H), 6.57 (s, 1H) | Calculated m/z 461.0, found m/z 461.0 |
| 23 | 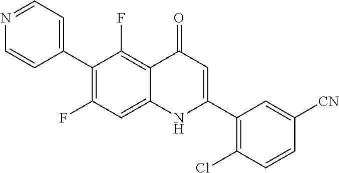 [SMILES: N#Cc1ccc(Cl)c(-c2cc(=O)c3c(F)c(-c4ccncc4)c(F)cc3[nH]2)c1] | 4-chloro-3-(5,7- difluoro-4-oxo-6- (pyridin-4-yl)-1,4- dihydroquinolin- 2-yl)benzonitrile | (400 MHz, DMSO-d₆) δ 8.73 (br d, J = 5.6 Hz, 2H), 8.24 (d, J = 1.8 Hz, 1H), 8.18 (s, 1H), 8.08 (dd, J = 2.0, 8.4 Hz, 1H), 7.93 (d, J = 8.4 Hz, 1H), 7.55 (br d, J = 5.2 Hz, 2H), 7.29 (br d, J = 10.8 | Calculated m/z 394.1, found m/z 394.0 |
| | | | Hz, 1H), 6.16 (br | |
| | | | s, 1H). | |
| 24 | 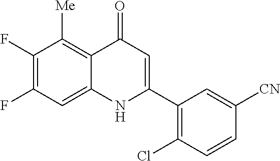 [SMILES: Cc1c(F)c(F)cc2[nH]c(-c3cc(C#N)ccc3Cl)cc(=O)c12] | 4-chloro-3-(6,7- difluoro-5- methyl-4-oxo-1,4- dihydroquinolin- 2-yl)benzonitrile | (400 MHz, DMSO-d₆) δ 11.95 (s, 1H), 8.22 (d, J = 1.8 Hz, 1H), 8.08 (dd, J = 1.8, 8.4 Hz, 1H), 7.92 (d, J = 8.4 Hz, 1H), 7.33 (dd, J = 7.2, 10.8 Hz, 1H), 6.05 (s, 1H), 2.80 (d, J = 2.8 Hz, 3H) | Calculated m/z 331.0, found m/z 331.0 |
| 25 | 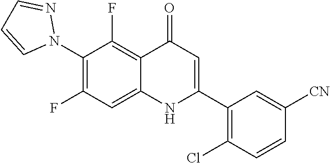 [SMILES: N#Cc1ccc(Cl)c(-c2cc(=O)c3c(F)c(-n4cccn4)c(F)cc3[nH]2)c1] | 4-chloro-3-(5,7- difluoro-4-oxo-6- (1H-pyrazol-1- yl)-1,4- dihydroquinolin- 2-yl)benzonitrile | (400 MHz, DMSO-d₆) δ = 12.36 (s, 1H), 8.27- 8.23 (m, 1H), 8.14-8.11 (m, 1H), 8.11-8.06 (m, 1H), 7.94 (d, J = 8.6 Hz, 1H), 7.84 (d, J = 1.6 Hz, 1H), 7.36 (br d, J = 10.6 Hz, | Calculated m/z 383.0, found m/z 383.0 |
| | | | 1H), 6.60-6.56 | |
| | | | (m, 1H), 6.20 (br | |
| | | | s, 1H) | |
| 26 | 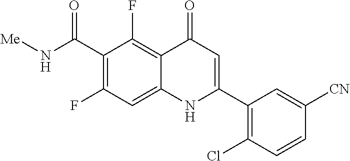 [SMILES: CNC(=O)c1c(F)cc2[nH]c(-c3cc(C#N)ccc3Cl)cc(=O)c2c1F] | 2-(2-chloro-5- cyanophenyl)-5,7- difluoro-N- methyl-4-oxo-1,4- dihydroquinoline- 6-carboxamide | (400 MHz, METHANOL-d4) δ 8.07 (d, J = 1.8 Hz, 1H), 7.97- 7.91 (m, 1H), 7.84 (d, J = 8.4 Hz, 1H), 7.15 (br d, J = 9.8 Hz, 1H), 6.27 (br s, 1H), 2.95 (s, 3H) | Calculated m/z 374.0, found m/z 373.9 |
| 27 | 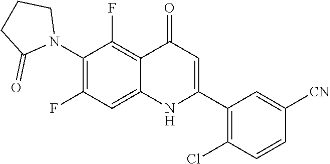 [SMILES: N#Cc1ccc(Cl)c(-c2cc(=O)c3c(F)c(N4CCCC4=O)c(F)cc3[nH]2)c1] | 4-chloro-3-(5,7- difluoro-4-oxo-6- (2-oxopyrrolidin- 1-yl)-1,4- dihydroquinolin- 2-yl)benzonitrile | (400 MHz, DMSO-d₆) δ 12.32- 12.07 (m, 1H), 8.22 (s, 1H), 8.07 (br d, J = 7.0 Hz, 1H), 7.91 (d, J = 8.4 Hz, 1H), 7.26- 7.13 (m, 1H), 6.06 (s, 1H), 3.87-3.54 (m, 3H), 2.24- 2.09 (m, 3H) | Calculated m/z 400.1, found m/z 400.0 |
| 28 | 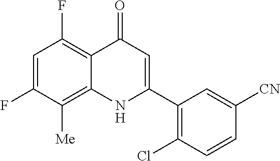 [SMILES: Cc1c(F)cc(F)c2c(=O)cc(-c3cc(C#N)ccc3Cl)[nH]c12] | 4-chloro-3-(5,7- difluoro-8- methyl-4-oxo-1,4- dihydroquinolin- 2-yl)benzonitrile | (400 MHz, METHANOL-d4) δ 8.06 (d, J = 2.0 Hz, 1H), 7.91 (br d, J = 7.8 Hz, 1H), 7.80 (d, J = 8.4 Hz, 1H), 7.00 (br t, J = 10.8 Hz, 1H), 6.34-6.17 (m, 1H), 2.40 (br s, 3H) | Calculated m/z 331.0, found m/z 330.9 |
| 29 | 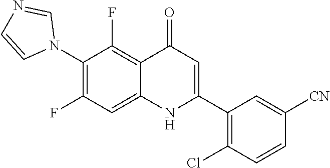 [SMILES: N#Cc1ccc(Cl)c(-c2cc(=O)c3c(F)c(-n4ccnc4)c(F)cc3[nH]2)c1] | 4-chloro-3-(5,7- difluoro-6-(1H- imidazol-1-yl)-4- oxo-1,4- dihydroquinolin- 2-yl)benzonitrile | (400 MHz, METHANOL-d₄) δ 9.51 (s, 1H), 8.09 (d, J = 1.8 Hz, 1H), 8.04 (s, 1H), 7.97 (dd, J = 1.8, 8.3 Hz, 1H), 7.92 (s, 1H), 7.86 (d, J = 8.4 Hz, 1H), 7.46 (dd, J = 1.5, 10.4 Hz, 1H), 6.42- | Calculated m/z 383.0, found m/z 383.0 |
| | | | 6.38 (m, 1H) | |
| 30 | 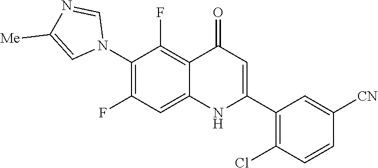 [SMILES: Cc1cn(-c2c(F)cc3[nH]c(-c4cc(C#N)ccc4Cl)cc(=O)c3c2F)cn1] | 4-chloro-3-(5,7- difluoro-6-(4- methyl-1H- imidazol-1-yl)-4- oxo-1,4- dihydroquinolin- 2-yl)benzonitrile | (400 MHz, METHANOL-d₄) δ 9.35 (s, 1H), 8.09 (d, J = 1.8 Hz, 1H), 7.97 (dd, J = 2.0, 8.4 Hz, 1H), 7.89-7.83 (m, 1H), 7.71 (s, 1H), 7.43 (dd, J = 1.8, 10.6 Hz, 1H), 6.39 (s, 1H), 2.49 (s, 3H) | Calculated m/z 397.1, found m/z 397.0 |

### Example 31

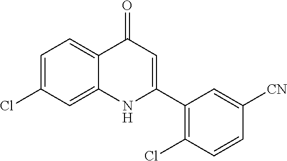 [SMILES: N#Cc1ccc(Cl)c(-c2cc(=O)c3ccc(Cl)cc3[nH]2)c1]

### 4-chloro-3-(7-chloro-4-oxo-1,4-dihydroquinolin-2-yl)benzonitrile

### Step 1, 1-(2-amino-4-chlorophenyl)ethan-1-one

To a solution of 1-(4-chloro-2-nitrophenyl)ethan-1-one (1.0 g, 5.0 mmol, 1.0 equiv.) in EtOH (9 mL) and H₂O (3 mL) was added iron (powder) (1.4 g, 25.1 mmol, 5.0 equiv.) and NH₄Cl (1.3 g, 25.1 mmol, 5.0 equiv.). The mixture was stirred at 80° C. for 16 hours. The reaction mixture was filtered and concentrated under reduced pressure to give a residue. The residue was diluted with water (20 mL) and extracted with EtOAc (3×20 mL). The combined organic layers were washed with brine (50 mL), dried over Na₂SO₄, filtered and concentrated under reduced pressure to give a residue. The residue was purified by silica gel column chromatography (0-50% ethyl acetate in petroleum ether) to afford the title compound (610 mg, 72% yield) as a yellow solid. ¹H NMR (400 MHz, CHLOROFORM-d) δ 7.64 (d, J=8.6 Hz, 1H), 6.66 (d, J=1.8 Hz, 1H), 6.62 (dd, J=1.8, 8.6 Hz, 1H), 6.36 (br s, 2H), 2.56 (s, 3H).

### Step 2, N-(2-acetyl-5-chlorophenyl)-2-chloro-5-cyanobenzamide

2-chloro-5-cyanobenzoic acid (136 mg, 749 μmol, 1.0 equiv.) was treated with SOCl₂ (1 mL) at 20° C. under N₂. The mixture was stirred at 80° C. for 1 hour. The mixture was concentrated under reduced pressure to give 2-chloro-5-cyano-benzoyl chloride (150 mg, crude) as a white solid. To a solution of 1-(2-amino-4-chlorophenyl)ethan-1-one (100 mg, 590 μmol, 1.0 equiv.) in DCM (2 mL) was added pyridine (119 uL, 1.5 mmol, 2.5 equiv.) and 2-chloro-5-cyano-benzoyl chloride (142 mg, 708 μmol, 1.2 equiv.). The mixture was stirred at 40° C. for 4 hours. The reaction mixture was diluted with water (20 mL) and extracted with DCM (3×20 mL). The combined organic layers were washed with HCl (1 N, 30 mL) and brine (50 mL), dried over Na₂SO₄, filtered, and concentrated under reduced pressure. The crude product was triturated with ACN (5 mL) at 20° C. for 30 minutes to afford the title compound (120 mg, 61% yield) as a white solid. LCMS [M+1]=333.0. ¹H NMR (400 MHz, DMSO-d₆) δ 11.78 (s, 1H), 8.43 (d, J=1.9 Hz, 1H), 8.25 (d, J=1.9 Hz, 1H), 8.08 (dd, J=1.6, 3.6 Hz, 2H), 7.87 (d, J=8.4 Hz, 1H), 7.45-7.40 (m, 1H), 2.63 (s, 3H).

### Step 3, 4-chloro-3-(7-chloro-4-oxo-1,4-dihydroquinolin-2-yl)benzonitrile

To a mixture of N-(2-acetyl-5-chlorophenyl)-2-chloro-5-cyanobenzamide (150 mg, 450 μmol, 1.0 equiv.) in dioxane (2 mL) was added LiGH (10.8 mg, 450 μmol, 1.0 equiv.) in one portion at 20° C. under N₂. The mixture was stirred at 110° C. for 5 hours. The mixture was poured into HCl (1 N) to adjust the pH to 5. Then the residue was poured into water (20 mL) and stirred for 30 minutes and then the mixture was filtered. The filter cake was triturated with ethyl acetate at 20° C. for 30 minutes and the precipitate was filtered. The filter cake was triturated with DMSO for 30 minutes to afford the title compound (68 mg, 48% yield). LCMS [M+1]=314.9/316.9. ¹H NMR (400 MHz, DMSO-d₆) δ 12.09 (br s, 1H), 8.26 (s, 1H), 8.11 (br dd, J=8.9, 13.5 Hz, 2H), 7.93 (d, J=8.3 Hz, 1H), 7.59 (br s, 1H), 7.47-7.36 (m, 1H), 6.15 (br s, 1H).

### Example 12

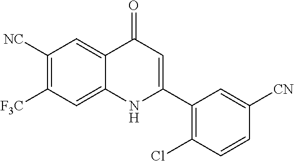 [SMILES: N#Cc1ccc(Cl)c(-c2cc(=O)c3cc(C#N)c(C(F)(F)F)cc3[nH]2)c1]

### 2-(2-chloro-5-cyanophenyl)-4-oxo-7-(trifluoromethyl)-1,4-dihydroquinoline-6-carbonitrile

### Scheme 3, Step 1. 2-chloro-5-cyano-N-(4-cyano-2-iodo-5-(trifluoromethyl)phenyl)benzamide

To a solution of 4-amino-5-iodo-2-(trifluoromethyl) benzonitrile (250 mg, 801 mol, 1.0 equiv.) in THF (1 mL) was added NaH (32.0 mg, 801 μmol, 60% dispersion in oil, 1.0 equiv.) at 0° C. The mixture was stirred for 10 minutes, and then 2-chloro-5-cyanobenzoyl chloride (160 mg, 801 μmol, 1.0 equiv.) was added. The mixture was warmed to room temperature and then stirred for 16 hours. The reaction was quenched with saturated aqueous NH₄Cl solution (10 mL) and then extracted with EtOAc (3×10 mL). The combined organic layers were washed with brine (10 mL), dried over anhydrous Na₂SO₄, filtered and concentrated under reduced pressure. The residue was purified by preparative TLC (SiO₂, petroleum ether:EtOAc=4:1) to afford the title compound (100 mg, 26% yield) as a white solid. MS mass calculated for [M−H]⁺ (C₁₆H₆ClF₃IN₃O) requires m/z=473.9, LCMS found m/z=474.0. ¹H NMR (400 MHz, CDCl₃) δ 8.37 (s, 1H), 7.93 (d, J=1.6 Hz, 1H), 7.88 (s, 1H), 7.68-7.62 (m, 1H), 7.52 (d, J=8.4 Hz, 1H).

### Scheme 3, step 2. 2-chloro-5-cyano-N-(4-cyano-2-(1-ethoxyvinyl)-5-(trifluoromethyl)phenyl)benzamide

To a solution of 2-chloro-5-cyano-N-(4-cyano-2-iodo-5-(trifluoromethyl)phenyl)benzamide (25 mg, 52 μmol, 1.0 equiv.) and tributyl(1-ethoxyvinyl)stannane (23.9 mg, 66.2 μmol, 1.26 equiv.) in toluene (1 mL) was added Pd(PPh₃)₄ (6.0 mg, 5.2 μmol, 0.1 equiv.) under an atmosphere of nitrogen. The mixture was stirred at 120° C. for 16 hours. The mixture was poured into an aqueous solution of KF (50 mL) and stirred for 1 hour. Then the mixture was diluted with water (10 mL) and extracted with EtOAc (3×20 mL). The combined organic layers were washed with brine (20 mL), dried over sodium sulfate, and concentrated under reduced pressure to afford the title compound (18 mg, crude) as a white solid. The crude product was used directly in next step. LCMS: calculated for [M−H]⁻ (C₂₀H₁₃ClF₃N₃O₂) requires m/z=418.1, LCMS found m/z=418.1.

### Scheme 3, step 3. N-(2-acetyl-4-cyano-5-(trifluoromethyl) phenyl)-2-chloro-5-cyanobenzamide

A solution of 2-chloro-5-cyano-N-(4-cyano-2-(1-ethoxyvinyl)-5-(trifluoromethyl) phenyl) benzamide (20 mg, 47.6 μmol, 1.0 equiv.) in HCl/dioxane (2 mL) was stirred at 20° C. for 1 hour. The reaction mixture was concentrated under reduced pressure and the residue was purified by preparative TLC (SiO₂, petroleum ether:EtOAc=2:1) to afford the title compound (8.0 mg, 43% yield) as a white solid. LCMS: calculated for [M−H]—(C₁₈H₉ClF₃N₃O₂) requires m/z=390.0, LCMS found m/z=390.1. ¹H NMR (400 MHz, CDCl₃) δ 12.49-12.60 (m, 1H), 9.46 (s, 1H), 8.40 (s, 1H), 7.95 (d, J=2.0 Hz, 1H), 7.77 (dd, J=8.40, 2.0 Hz, 1H), 7.65-7.69 (m, 1H), 2.78 (s, 3H).

### Scheme 3, step 4. 2-(2-chloro-5-cyanophenyl)-4-oxo-7-(trifluoromethyl)-1,4-dihydroquinoline-6-carbonitrile

To a mixture of N-(2-acetyl-4-cyano-5-(trifluoromethyl)phenyl)-2-chloro-5-cyanobenzamide (50 mg, 128 μmol, 1.0 equiv.), NaOH (51 mg, 1.28 mmol, 10 equiv.) in dioxane (1 mL) was degassed and purged with nitrogen 3 times, and then the mixture was stirred at 110° C. for 1 hour under an atmosphere of nitrogen. The pH of the solution was adjusted to 5-6 with aqueous HCl (1M) and then extracted with EtOAc (3×5 mL). The combined organic layers were washed with brine (5 mL), dried over anhydrous Na₂SO₄, filtered, and concentrated under reduced pressure. The residue was purified by preparative HPLC (column: Waters Xbridge BEH C18 100×30 mm×10 m; mobile phase: [water (10 mM NH₄HCO₃)-ACN]; B %: 20%-50%, 8 min) to afford the title compound (6.5 mg, 14% yield, 99% purity) as a yellow solid. LCMS: calculated for [M−H]⁻ (C₁₈H₇ClF₃N₃₀) requires m/z=372.0, LCMS found m/z=372.0. ¹H NMR (400 MHz, DMSO-d₆) δ 8.64 (s, 1H), 8.08 (s, 1H), 8.00 (s, 1H), 7.96 (br d, J=8.2 Hz, 1H), 7.82 (d, J=8.4 Hz, 1H), 6.37 (s, 1H).

The compounds in Table 3 were prepared following Scheme 3 using similar procedures to those described for Example 12.

TABLE 3

| | | | | LCMS |
|---|---|---|---|---|
| Example | | | | Exact |
| number | Structure | IUPAC Name | ¹H NMR | Mass |
| 32 | 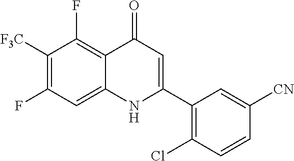 [SMILES: N#Cc1ccc(Cl)c(-c2cc(=O)c3c(F)c(C(F)(F)F)c(F)cc3[nH]2)c1] | 4-chloro-3-(5,7- difluoro-4-oxo- 6- (trifluoromethyl)- 1,4- dihydroquinolin- 2-yl)benzonitrile | (400 MHz, DMSO-d₆) δ 12.46 (br s, 1H), 8.23 (d, J = 1.8 Hz, 1H), 8.09 (dd, J = 1.8, 8.4 Hz, 1H), 7.93 (d, J = 8.6 Hz, 1H), 7.29 (br d, J = 12.0 Hz, 1H), 6.22 (s, 1H). | Calculated m/z 385.0, found m/z 385.0 |
| 33 | 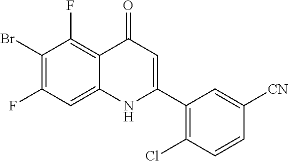 [SMILES: N#Cc1ccc(Cl)c(-c2cc(=O)c3c(F)c(Br)c(F)cc3[nH]2)c1] | 3-(6-bromo-5,7- difluoro-4-oxo- 1,4- dihydroquinolin- 2-yl)-4- chlorobenzo- nitrile | (400 MHz, DMSO-d₆) δ 12.26 (s, 1H), 8.25 (br s, 1H), 8.10 (br d, J = 8.1 Hz, 1H), 7.94 (d, J = 8.4 Hz, 1H), 7.38- 7.23 (m, 1H), 6.15 (br s, 1H) | Calculated m/z 394.9, found m/z 394.9 |
| 34 | 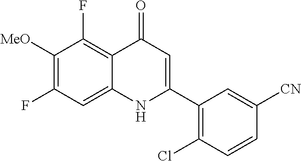 [SMILES: COc1c(F)cc2[nH]c(-c3cc(C#N)ccc3Cl)cc(=O)c2c1F] | 4-chloro-3-(5,7- difluoro-6- methoxy-4-oxo- 1,4- dihydroquinolin- 2-yl)benzonitrile | (400 MHz, DMSO-d₆) δ = 12.05 (br s, 1H), 8.22 (br s, 1H), 8.11-8.04 (m, 1H), 7.91 (d, J = 8.4 Hz, 1H), 7.39- 7.06 (m, 1H), 6.23- 5.93 (m, 1H), 3.94 (s, 3H) | Calculated m/z 347.0, found m/z 347.0 |

### Example 35

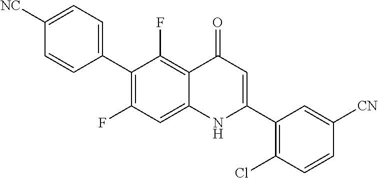 [SMILES: N#Cc1ccc(-c2c(F)cc3[nH]c(-c4cc(C#N)ccc4Cl)cc(=O)c3c2F)cc1]

### 4-chloro-3-(6-(4-cyanophenyl)-5,7-difluoro-4-oxo-1,4-dihydroquinolin-2-yl)benzonitrile

1-(6-amino-3-bromo-2,4-difluorophenyl)ethan-1-one: To a mixture of 1-(2-amino-4,6-difluoro-phenyl)ethanone (40 g, 233.7 mmol, 1 equiv.) in DCM (400 mL) was added N-bromosuccinimide (45.8 g, 257 mmol, 1.1 equiv.) at 20° C. under N₂. The mixture was stirred at 20° C. for 2 hours. The reaction mixture was poured into water (50 mL) and extracted with DCM (3×200 mL). The combined organic layers were washed with brine (2×50 mL), dried with anhydrous Na₂SO₄, filtered, and concentrated under reduced pressure. The crude product was purified by silica gel column chromatography (4% to 8% ethyl acetate in petroleum ether) to afford the title compound (29.9 g, 47% yield) as yellow solid. LCMS [M+1]=249.9. ¹H NMR (400 MHz, CHLOROFORM-d) δ 6.71-6.46 (m, 2H), 6.26 (dd, J=1.8, 10.2 Hz, 1H), 2.60 (d, J=8.6 Hz, 3H).

### Step 1, Scheme 4, 3′-acetyl-4′-amino-2′,6′-difluoro-[1,1′-biphenyl]-4-carbonitrile

To a solution of (4-cyanophenyl)boronic acid (176 mg, 1.2 mmol, 1.5 equiv.) and 1-(6-amino-3-bromo-2,4-difluorophenyl)ethan-1-one (200 mg, 800 μmol, 1.0 equiv.) in dioxane (1 mL) and H₂O (0.33 mL) was added K₂CO₃ (332 mg, 2.40 mmol, 3 equiv.) and Pd(dppf)Cl₂—CH₂Cl₂ (65 mg, 80 μmol, 0.1 equiv.) under N₂. The mixture was stirred at 80° C. for 16 hours under N₂. The reaction mixture was diluted with water (10 mL) and extracted with ethyl acetate (15 mL). The organic layer was washed with brine (10 mL), dried over Na₂SO₄, filtered, and concentrated under reduced pressure. The residue was purified by preparative TLC (25% ethyl acetate in petroleum ether) to afford the title compound (200 mg, 92% yield) as a white solid. LCMS [M+1]=273.1. ¹H NMR (400 MHz, CHLOROFORM-d) δ 2.60 (d, J=9.0 Hz, 3H), 6.28 (dd, J=11.6, 1.6 Hz, 1H), 6.38-6.82 (m, 2H), 7.53 (br d, J=8.4 Hz, 2H), 7.68-7.77 (m, 2H).

### Step 2, N-(3-acetyl-4′-cyano-2,6-difluoro-[1,1′-biphenyl]-4-yl)-2-chloro-5-cyanobenzamide

To a solution of 3′-acetyl-4′-amino-2′,6′-difluoro-[1,1′-biphenyl]-4-carbonitrile (200 mg, 734.6 μmol, 1.0 equiv.) in THF (2 mL) was added NaH (29.4 mg, 735 μmol, 1.0 equiv.; 60% dispersion in oil). Then 2-chloro-5-cyanobenzoyl chloride (220 mg, 1.10 mmol, 1.5 equiv.) was added and the mixture was stirred at 0-20° C. for 16 hours. The reaction was quenched with saturated aqueous ammonium chloride solution (5 mL) and then extracted with ethyl acetate (20 mL). The organic layer was concentrated under reduced pressure to give a residue. The crude product was triturated with ethyl acetate to afford the title compound (220 mg, 69% yield) as a white solid. LCMS [M+1]=436.0. ¹H NMR (400 MHz, CHLOROFORM-d) δ 12.22 (s, 1H), 8.66 (dd, J=12.2, 1.6 Hz, 1H), 7.94 (d, J=1.8 Hz, 1H), 7.77-7.86 (m, 3H), 7.71-7.77 (m, 1H), 7.62-7.68 (m, 1H), 7.59 (br d, J=8.2 Hz, 2H), 2.70 (d, J=8.8 Hz, 3H).

### Step 3, 4-chloro-3-(6-(4-cyanophenyl)-5,7-difluoro-4-oxo-1,4-dihydroquinolin-2-yl)benzonitrile

To a solution of N-(3-acetyl-4′-cyano-2,6-difluoro-[1,1′-biphenyl]-4-yl)-2-chloro-5-cyanobenzamide (100 mg, 229 μmol, 1.0 equiv.) in dioxane (2 mL) was added NaOH (9.2 mg, 229 μmol, 1.0 equiv.). The mixture was stirred at 110° C. for 1.5 hours. The reaction was quenched by the addition of aqueous HCl (1M), and then diluted with water (5 mL). The mixture was extracted with ethyl acetate (3×10 mL). The combined organic layers were washed with brine (20 mL), dried over Na₂SO₄, filtered, and concentrated under reduced pressure. The residue was purified by preparative HPLC (column: Phenomenex Luna C18 150×30 mm×5 um; mobile phase: [25-65% acetonitrile in water (+0.2% formic acid)) to afford the title compound (22 mg, 23% yield) as a white solid. LCMS [M+1]=418.0. ¹H NMR (400 MHz, DMSO-d₆) δ ppm 12.25 (br d, J=1.8 Hz, 1H), 8.24 (d, J=1.8 Hz, 1H), 8.08 (dd, J=8.4, 1.8 Hz, 1H) 8.00 (d, J=8.4 Hz, 2H), 7.93 (d, J=8.4 Hz, 1H), 7.73 (d, J=8.0 Hz, 2H), 7.28 (br d, J=10.8 Hz, 1H), 6.15 (br s, 1H).

The compounds in Table 4 were prepared following Scheme 4 using similar procedures as those described for Example 35.

TABLE 4

| | | | | LCMS |
|---|---|---|---|---|
| Example number | Structure | IUPAC Name | ¹H NMR | Exact Mass |
| 36 | 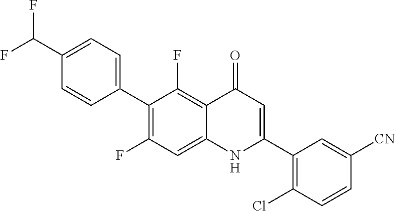 [SMILES: N#Cc1ccc(Cl)c(-c2cc(=O)c3c(F)c(-c4ccc(C(F)F)cc4)c(F)cc3[nH]2)c1] | 4-chloro-3-(6-(4- (difluoromethyl) phenyl)-5,7- difluoro-4-oxo-1,4- dihydroquinolin- 2-yl)benzonitrile | (400 MHz, DMSO-d₆) δ 12.21 (s, 1H), 8.27 (d, J = 1.6 Hz, 1H), 8.11 (dd, J = 1.8, 8.4 Hz, 1H), 7.95 (d, J = 8.4 Hz, 1H), 7.73 (br d, J = 8.0 Hz, 2H), 7.65 (br d, J = 7.8 Hz, 2H), 7.31-7.22 (m, 1H), 7.13 (s, 1H), 6.99 (s, 1H). | Calculated m/z 443.0, found m/z 443.0 |
| 37 | 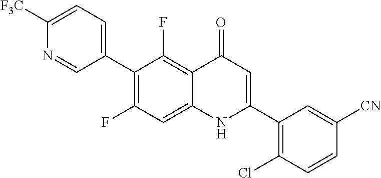 [SMILES: N#Cc1ccc(Cl)c(-c2cc(=O)c3c(F)c(-c4ccc(C(F)(F)F)nc4)c(F)cc3[nH]2)c1] | 4-chloro-3-(5,7- difluoro-4-oxo-6- (6-(trifluoromethyl) pyridin-3-yl)-1,4- dihydroquinolin- 2-yl)benzonitrile | (400 MHz, DMSO-d₆) δ 12.28 (br s, 1 H) 8.94 (br s, 1 H) 8.20-8.37 (m, 2 H) 8.10 (br d, J = 8.2 Hz, 2 H) 7.94 (br d, J = 8.4 Hz, 1 H) 7.33 (br d, J = 8.8 Hz, 1 H) 6.16 (br s, 1 H) | Calculated m/z 462.0, found m/z 462.0 |
| 38 | 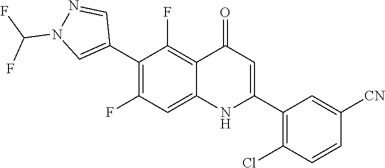 [SMILES: N#Cc1ccc(Cl)c(-c2cc(=O)c3c(F)c(-c4cnn(C(F)F)c4)c(F)cc3[nH]2)c1] | 4-chloro-3-(6-(1- (difluoromethyl)- 1H-pyrazol-4-yl)- 5,7-difluoro-4- oxo-1,4- dihydroquinolin- 2-yl)benzonitrile | (400 MHz, DMSO-d₆) δ 12.18 (s, 1H), 8.67 (s, 1H), 8.26 (d, J = 1.6 Hz, 1H), 8.20 (s, 1H), 8.13-7.75 (m, 3H), 7.26 (br d, J = 11.4 Hz, 1H), 6.10 (s, 1H). | Calculated m/z 433.0, found m/z 432.9 |
| 39 | 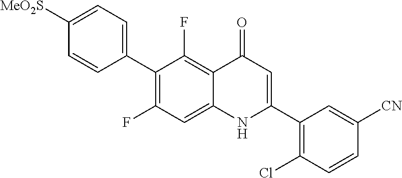 [SMILES: CS(=O)(=O)c1ccc(-c2c(F)cc3[nH]c(-c4cc(C#N)ccc4Cl)cc(=O)c3c2F)cc1] | 4-chloro-3- (5,7-difluoro-6-(4- (methylsulfonyl) phenyl)-4-oxo- 1,4- dihydroquinolin- 2-yl)benzonitrile | (400 MHz, DMSO-d₆) δ 12.24 (br d, J = 2.0 Hz, 1H), 8.26 (d, J = 1.8 Hz, 1H), 8.12-8.06 (m, 3H), 7.94 (d, J = 8.4 Hz, 1H), 7.80 (d, J = 8.2 Hz, 2H), 7.29 (br d, J = 7.8 Hz, | Calculated m/z 471.0, found m/z 471.0 |
| | | | 1H), 6.13 (br s, | |
| | | | 1H), 3.32-3.31 | |
| | | | (m, 3H). | |
| 40 | 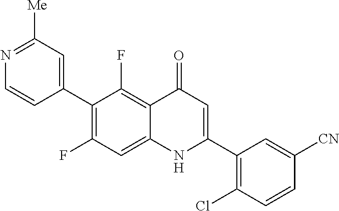 [SMILES: Cc1cc(-c2c(F)cc3[nH]c(-c4cc(C#N)ccc4Cl)cc(=O)c3c2F)ccn1] | 4-chloro-3- (5,7-difluoro- 6-(2- methylpyridin- 4-yl)-4-oxo- 1,4- dihydroquinolin- 2-yl)benzonitrile | (400 MHz, DMSO-d₆) δ 12.43-12.10 (m, 1H), 8.59 (d, J = 5.0 Hz, 1H), 8.26 (br d, J = 1.6 Hz, 1H), 8.16-8.05 (m, 1H), 7.94 (d, J = 8.6 Hz, 1H), 7.41 (s, 1H), 7.36- 7.24 (m, 1H), 6.33- 5.98 (m, 2H), 2.57- 2.52 (m, 3H). | Calculated m/z 408.1, found m/z 408.0 |
| 41 | 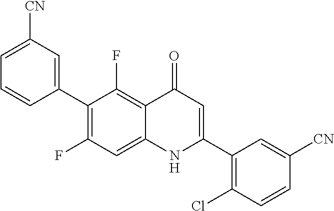 [SMILES: N#Cc1cccc(-c2c(F)cc3[nH]c(-c4cc(C#N)ccc4Cl)cc(=O)c3c2F)c1] | 4-chloro-3-(6- (3- cyanophenyl)-5,7- difluoro-4-oxo- 1,4- dihydroquinolin- 2-yl)benzonitrile | (400 MHz, DMSO-d₆) δ 12.24 (br s, 1H), 8.26 (br d, J = 1.4 Hz, 1H), 8.10 (br dd, J = 2.0, 8.4 Hz, 1H), 8.04 (s, 1H), 7.98-7.92 (m, 2H), 7.87 (br d, J = 7.8 Hz, 1H), 7.75 (br t, J = 7.8 Hz, 1H), 7.35-7.19 (m, | Calculated m/z 418.1, found m/z 418.0 |
| | | | 1H), 6.24-6.03 (m, | |
| | | | 1H) | |
| 42 | 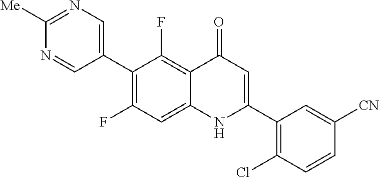 [SMILES: Cc1ncc(-c2c(F)cc3[nH]c(-c4cc(C#N)ccc4Cl)cc(=O)c3c2F)cn1] | 4-chloro-3- (5,7-difluoro-6- (2- methylpyrimidin- 5-yl)-4-oxo-1,4- dihydroquinolin- 2-yl)benzonitrile | (400 MHz, DMSO-d₆) δ 12.25 (s, 1H), 7.87 (s, 2H), 8.26 (s, 1H), 8.09 (d, J = 6.8 Hz, 1H), 7.93 (d, J = 8.4 Hz, 1H), 7.30-7.28 (m, 1H), 6.13 (s, 1H), 2.71 (s, 3H) | Calculated m/z 409.1, found m/z 409.0 |
| 43 | 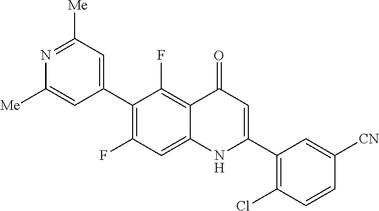 [SMILES: Cc1cc(-c2c(F)cc3[nH]c(-c4cc(C#N)ccc4Cl)cc(=O)c3c2F)cc(C)n1] | 4-chloro-3-(6- (2,6- dimethylpyridin- 4-yl)-5,7- difluoro-4- oxo-1,4- dihydroquinolin- 2-yl)benzonitrile | (400 MHz, DMSO-d₆) δ 12.24 (br s, 1H), 8.25 (s, 1H), 8.15- 8.06 (m, 1H), 7.98-7.90 (m, 1H), 7.31-6.98 (m, 1H), 7.18 (s, 2H), 6.11 (s, 1H), 2.53-2.52 (m, 6H) | Calculated m/z 422.1, found m/z 422.0 |
| 44 | 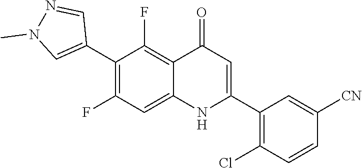 [SMILES: Cn1cc(-c2c(F)cc3[nH]c(-c4cc(C#N)ccc4Cl)cc(=O)c3c2F)cn1] | 4-chloro-3- (5,7-difluoro- 6-(1-methyl- 1H-pyrazol-4- yl)-4-oxo-1,4- dihydroquinolin- 2-yl)benzonitrile | (400 MHz, DMSO-d₆) δ 12.11 (s, 1H), 8.25 (d, J = 2.0 Hz, 1H), 8.20 (s, 1H), 8.09 (dd, J = 2.0, 8.4 Hz, 1H), 7.93 (d, J = 8.4 Hz, 1H), 7.85 (s, 1H), 7.22 (d, J = 11.4 Hz, 1H), 6.07 (s, 1H), 3.93 (s, 3H). | Calculated m/z 397.1, found m/z 397.0 |
| 45 | 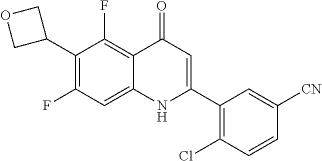 [SMILES: N#Cc1ccc(Cl)c(-c2cc(=O)c3c(F)c(C4COC4)c(F)cc3[nH]2)c1] | 4-chloro-3- (5,7-difluoro- 6-(oxetan-3- yl)-4-oxo-1,4- dihydroquinolin- 2-yl)benzonitrile | (400 MHz, DMSO-d₆) δ 12.09 (br s, 1H), 8.23 (s, 1H), 8.12- 8.04 (m, 1H), 7.92 (br d, J = 8.4 Hz, 1H), 7.13 (br d, J = 10.8 Hz, 1H), 6.05 (s, 1H), 4.88 (br d, J = 8.4 Hz, 4H), 4.67 | Calculated m/z 373.1, LCMS found m/z 373.0 |
| | | | (br d, J = 8.6 Hz, | |
| | | | 1H) | |
| 46 | 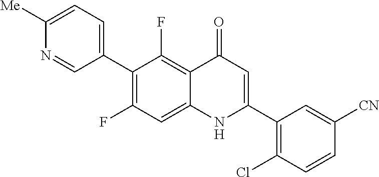 [SMILES: Cc1ccc(-c2c(F)cc3[nH]c(-c4cc(C#N)ccc4Cl)cc(=O)c3c2F)cn1] | 4-chloro-3- (5,7-difluoro- 6-(6- methylpyridin- 3-yl)-4-oxo- 1,4- dihydroquinolin- 2-yl)benzonitrile | (400 MHz, DMSO-d₆) δ = 8.54 (br s, 1H), 8.22 (br s, 1H), 8.06 (br d, J = 8.6 Hz, 1H), 7.91 (br d, J = 8.2 Hz, 1H), 7.82 (br d, J = 7.8 Hz, 1H), 7.42 (br d, J = 7.8 Hz, 1H), 7.27 (br | Calculated m/z 408.1, found m/z 408.0 |
| | | | d, J = 10.6 Hz, | |
| | | | 1H), 6.15 (br s, | |
| | | | 1H), 2.62-2.53 | |
| | | | (m, 3H) | |
| 47 | 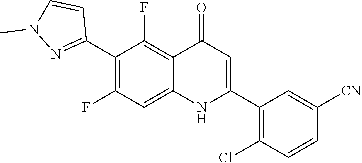 [SMILES: Cn1ccc(-c2c(F)cc3[nH]c(-c4cc(C#N)ccc4Cl)cc(=O)c3c2F)n1] | 4-chloro-3- (5,7-difluoro- 6-(1-methyl- 1H-pyrazol-3- yl)-4-oxo-1,4- dihydroquinolin- 2-yl)benzonitrile | (400 MHz, DMSO-d₆) δ 12.54-11.81 (m, 1H), 8.26 (d, J = 2.0 Hz, 1H), 8.09 (dd, J = 2.0, 8.4 Hz, 1H), 7.93 (d, J = 8.4 Hz, 1H), 7.85 (d, J = 2.2 Hz, 1H), 7.22 (br d, J = 10.4 Hz | Calculated m/z 397.1, found m/z 397.0 |
| | | | 1H), 6.54 (s, 1H), | |
| | | | 6.12 (br s, 1H), | |
| | | | 3.96-3.90 (m, 3H) | |
| 48 | 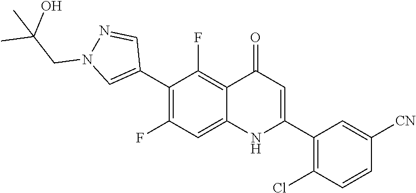 [SMILES: CC(C)(O)Cn1cc(-c2c(F)cc3[nH]c(-c4cc(C#N)ccc4Cl)cc(=O)c3c2F)cn1] | 4-chloro-3- (5,7-difluoro- 6-(1-(2- hydroxy-2- methylpropyl)- 1H-pyrazol-4- yl)-4-oxo-1,4- dihydroquinolin- 2-yl)benzonitrile | (400 MHz, DMSO-d₆) δ 12.78- 11.46 (m, 1H), 8.25 (d, J = 1.8 Hz, 1H), 8.16 (s, 1H), 8.09 (dd, J = 2.0, 8.4 Hz, 1H), 7.93 (d, J = 8.4 Hz, 1H), 7.88 (s, 1H), 7.27 (br d, J = 11.8 Hz, 1H), 6.14 (br s, 1H), 4.12 (s, 2H), 1.11 (s, 6H) | Calculated m/z 455.1, found m/z 455.0 |
| 49 | 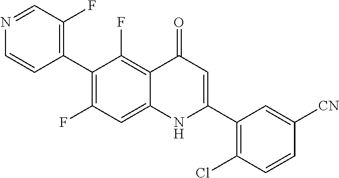 [SMILES: N#Cc1ccc(Cl)c(-c2cc(=O)c3c(F)c(-c4ccncc4F)c(F)cc3[nH]2)c1] | 4-chloro-3- (5,7-difluoro- 6-(3- fluoropyridin- 4-yl)-4-oxo- 1,4- dihydroquinolin- 2-yl)benzonitrile | (400 MHz, DMSO- d₆) δ 12.33 (br s, 1H), 8.80 (d, J = 1.2 Hz, 1H), 8.63-8.60 (m, 1H), 8.25 (d, J = 1.8 Hz, 1H), 8.10 (dd, J = 2.0, 8.4 Hz, 1H), 7.94 (d, J = 8.4 Hz, 1H), 7.69 (t, J = 5.6 Hz, 1H), 7.40-7.23 (m, 1H), 6.18 (br s, 1H) | Calculated m/z 412.0, found m/z 411.9 |
| 50 | 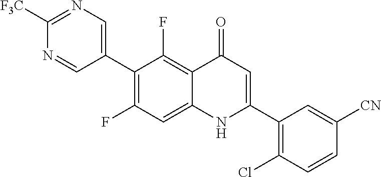 [SMILES: N#Cc1ccc(Cl)c(-c2cc(=O)c3c(F)c(-c4cnc(C(F)(F)F)nc4)c(F)cc3[nH]2)c1] | 4-chloro-3- (5,7-difluoro- 4-oxo-6-(2- (trifluoromethyl) pyrimidin-5-yl)- 1,4- dihydroquinolin- 2-yl)benzonitrile | (400 MHz, METHANOL-d₄) δ 9.22 (s, 2H), 8.11 (d, J = 2.0 Hz, 1H), 8.01-7.94 (m, 1H), 7.87 (d, J = 8.4 Hz, 1H), 7.39-7.29 (m, 1H), 6.38-6.29 (m, 1H) | Calculated m/z 463.0, found m/z 463.0 |
| 51 | 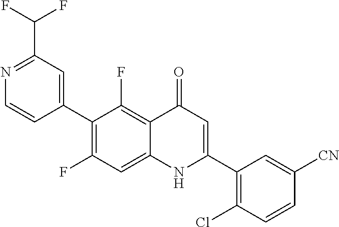 [SMILES: N#Cc1ccc(Cl)c(-c2cc(=O)c3c(F)c(-c4ccnc(C(F)F)c4)c(F)cc3[nH]2)c1] | 4-chloro-3-(6- (2- (difluoromethyl) pyridin-4-yl)- 5,7-difluoro-4- oxo-1,4- dihydroquinolin- 2-yl)benzonitrile | (400 MHz, DMSO-d₆) δ 12.29 (br dd, J = 2.2, 3.4 Hz, 1H), 8.86 (d, J = 5.0 Hz, 1H), 8.27 (s, 1H), 8.11 (dd, J = 1.8, 8.4 Hz, 1H), 7.95 (d, J = 8.4 Hz, 1H), 7.88 (s, 1H), 7.77 (br d, J = 4.6 Hz, 1H), 7.31 (br dd, J = 2.6, 6.4 Hz, 1H), 7.24-6.82 (m, | Calculated m/z 444.0, found m/z 444.0 |
| | | | 1H), 6.22-6.08 | |
| | | | (m, 1H) | |
| 52 | 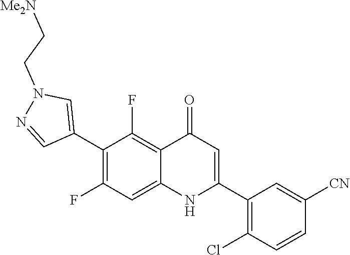 [SMILES: CN(C)CCn1cc(-c2c(F)cc3[nH]c(-c4cc(C#N)ccc4Cl)cc(=O)c3c2F)cn1] | 4-chloro-3-(6- (1-(2- (dimethylamino) ethyl)-1H- pyrazol-4-yl)-5,7- difluoro-4- oxo-1,4- dihydroquinolin- 2-yl)benzonitrile (hydrochloride salt) | (400 MHz, DMSO- d₆) δ 12.19 (br d, J = 0.6 Hz, 1H), 9.66-9.57 (m, 1H), 8.39 (br s, 1H), 8.24 (br s, 1H), 8.09 (br d, J = 8.2 Hz, 1H), 8.00 (br s, 1H), 7.94 (br d, J = 8.6 Hz, 1H), 7.34-7.21 (m, 1H), 6.13-6.03 (m, 1H), 4.65 (br t, J = 6.2 Hz, 2H), 3.73- 3.59 (m, 2H), 2.89- 2.75 (m, 6H) | Calculated m/z 454.0, found m/z 454.1 |
| 53 | 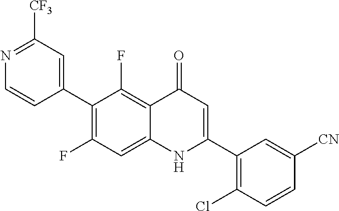 [SMILES: N#Cc1ccc(Cl)c(-c2cc(=O)c3c(F)c(-c4ccnc(C(F)(F)F)c4)c(F)cc3[nH]2)c1] | 4-chloro-3- (5,7-difluoro- 4-oxo-6-(2- (trifluoromethyl) pyridin-4-yl)-1,4- dihydroquinolin- 2-yl)benzonitrile | (400 MHz, DMSO-d₆) δ 12.32 (br s, 1H), 8.94 (d, J = 5.0 Hz, 1H), 8.26 (s, 1H), 8.15-8.05 (m, 2H), 7.93 (br d, J = 8.2 Hz, 2H), 7.31 (br d, J = 10.8 Hz, 1H), 6.18 (br s, 1H) | Calculated m/z 462.0, found m/z 461.9 |
| 54 | 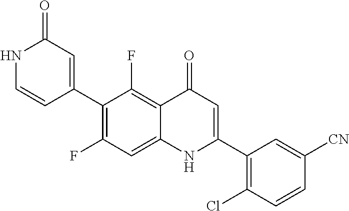 [SMILES: N#Cc1ccc(Cl)c(-c2cc(=O)c3c(F)c(-c4cc[nH]c(=O)c4)c(F)cc3[nH]2)c1] | 4-chloro-3- (5,7-difluoro- 4-oxo-6-(2- oxo-1,2- dihydropyridin- 4-yl)-1,4- dihydroquinolin- 2-yl)benzonitrile | (400 MHz, DMSO-d₆) δ 12.47-12.27 (m, 1H), 11.91-11.71 (m, 1H), 8.2 (d, J = 1.8 Hz, 1 H) 8.1 (dd, J = 8.4, 2.0 Hz, 1 H) 7.9 (d, J = 8.4 Hz, 1 H) 7.5 (d, J = 6.8 Hz, 1 H) 7.2-7.3 (m, 1 H) 6.5 (s, 1 H) 6.3 (br d, J = 6.6 Hz, 1 H) 6.1-6.2 (m, 1 H) | Calculated m/z 410.0, found m/z 409.9 |
| 55 | 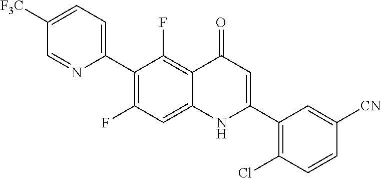 [SMILES: N#Cc1ccc(Cl)c(-c2cc(=O)c3c(F)c(-c4ccc(C(F)(F)F)cn4)c(F)cc3[nH]2)c1] | 4-chloro-3- (5,7-difluoro- 4-oxo-6-(5- (trifluoromethyl) pyridin-2-yl)-1,4- dihydroquinolin- 2-yl)benzonitrile | ¹H NMR (400 MHz, DMSO-d₆) δ 9.16 (s, 1H), 8.42 (br d, J = 8.8 Hz, 1H), 8.27 (s, 1H), 8.13-8.10 (m, 1H), 8.09 (s, 1H), 7.94 (br d, J = 8.2 Hz, 2H), 3.31-3.27 (m, 2H) | Calculated m/z 462.0, found m/z 462.0 |
| 56 | 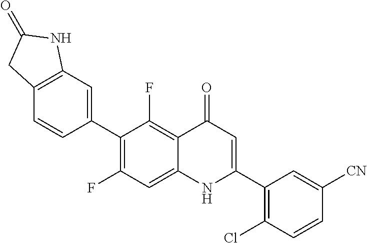 [SMILES: N#Cc1ccc(Cl)c(-c2cc(=O)c3c(F)c(-c4ccc5c(c4)NC(=O)C5)c(F)cc3[nH]2)c1] | 4-chloro-3- (5,7-difluoro- 4-oxo-6-(2- oxoindolin-6- yl)-1,4- dihydroquinolin- 2- yl)benzonitrile | (400 MHz, DMSO-d₆) δ 12.15 (br s, 1H), 10.49 (s, 1H), 8.26 (s, 1H), 8.10 (br d, J = 7.8 Hz, 1H), 7.94 (br d, J = 8.4 Hz, 1H), 7.34 (br d, J = 7.4 Hz, 1H), 7.22 (br d, J = 9.8 Hz, 1H), 7.02 (br d, J = 7.4 Hz, 1H), 6.88 (s, 1H), 6.09 (s, 1H), 3.55 (s, 2H) | Calculated m/z 448.1, found m/z 448.0 |
| 57 | 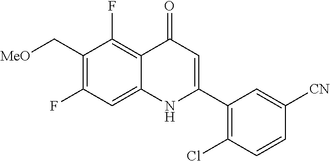 [SMILES: COCc1c(F)cc2[nH]c(-c3cc(C#N)ccc3Cl)cc(=O)c2c1F] | 4-chloro-3- (5,7-difluoro-6- (methoxymethyl)- 4-oxo-1,4- dihydroquinolin- 2-yl)benzonitrile | (400 MHz, DMSO- d₆) δ 12.13 (s, 1H), 8.25 (d, J = 1.8 Hz, 1H), 8.09 (dd, J = 2.0, 8.4 Hz, 1H), 7.93 (d, J = 8.4 Hz, 1H), 7.14 (d, J = 10.4 Hz, 1H), 6.07 (d, J = 1.4 Hz, 1H), 4.50 (s, 2H), 3.27 (s, 3H) | Calculated m/z 361.1, LCMS found m/z 360.9 |
| 58 | 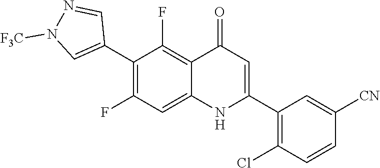 [SMILES: N#Cc1ccc(Cl)c(-c2cc(=O)c3c(F)c(-c4cnn(C(F)(F)F)c4)c(F)cc3[nH]2)c1] | 4-chloro-3- (5,7-difluoro- 4-oxo-6-(1- (trifluoromethyl)- 1H-pyrazol-4-yl)- 1,4- dihydroquinolin- 2-yl)benzonitrile | (400 MHz, DMSO- d₆) δ 12.2 (br s, 1H), 8.9 (s, 1H), 8.4 (s, 1H,) 8.3 (br d, J = 1.8 Hz, 1H), 8.1 (dd, J = 8.6, 2.0 Hz, 1H), 7.9 (d, J = 8.4 Hz, 1H), 7.3-7.3 (m, 1H), 6.1 (br d, J = 1.6 Hz, 1H) | Calculated m/z 451.0, found m/z 450.9 |
| 59 | 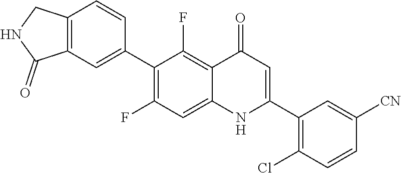 [SMILES: N#Cc1ccc(Cl)c(-c2cc(=O)c3c(F)c(-c4ccc5c(c4)C(=O)NC5)c(F)cc3[nH]2)c1] | 4-chloro-3- (5,7-difluoro- 4-oxo-6-(3- oxoisoindolin- 5-yl)-1,4- dihydroquinolin- 2-yl)benzonitrile | (400 MHz, DMSO- d₆) δ 12.21 (br s, 1H), 8.69 (s, 1H), 8.26 (s, 1H), 8.16-8.03 (m, 1H), 7.94 (d, J = 8.4 Hz, 1H), 7.83- 7.65 (m, 3H), 7.27 (br d, J = 8.2 Hz, 1H), 6.11 (br d, J = 1.6 Hz, 1H), 4.47 (s, 2H) | Calculated m/z 448.1, found m/z 448.0 |
| 60 | 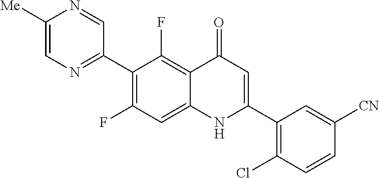 [SMILES: Cc1cnc(-c2c(F)cc3[nH]c(-c4cc(C#N)ccc4Cl)cc(=O)c3c2F)cn1] | 4-chloro-3- (5,7-difluoro- 6-(5- methylpyrazin- 2-yl)-4-oxo- 1,4- dihydroquinolin- 2-yl)benzonitrile | (400 MHz, DMSO- d₆) δ 12.26 (br s, 1H), 8.75 (br d, J = 11.0 Hz, 2H), 8.27 (br s, 1H), 8.10 (br d, J = 8.0 Hz, 1H), 7.94 (br d, J = 8.6 Hz, 1H), 7.27 (br d, J = 10.8 Hz, 1H), 6.13 (br s, 1H), 2.59 (br s, 3H) | Calculated m/z 409.1, found m/z 408.9 |
| 61 | 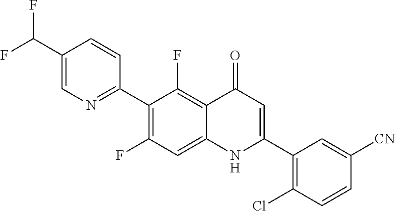 [SMILES: N#Cc1ccc(Cl)c(-c2cc(=O)c3c(F)c(-c4ccc(C(F)F)cn4)c(F)cc3[nH]2)c1] | 4-chloro-3-(6- (5- (difluoromethyl) pyridin-2-yl)- 5,7-difluoro-4- oxo-1,4- dihydroquinolin- 2-yl)benzonitrile | (400 MHz, DMSO- d₆) δ 8.95 (s, 1H), 8.27 (d, J = 2.0 Hz, 1H), 8.23-8.16 (m, 1H), 8.13-8.08 (m, 1H), 7.94 (d, J = 8.4 Hz, 1H), 7.83 (d, J = 8.2 Hz, 1H), 7.41- 7.09 (m, 2H), 6.18 (s, 1H) | Calculated m/z 440.0, found m/z 443.9 |

### Example 62

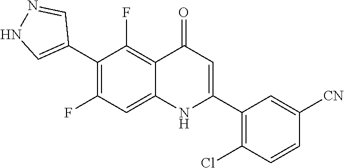 [SMILES: N#Cc1ccc(Cl)c(-c2cc(=O)c3c(F)c(-c4cn[nH]c4)c(F)cc3[nH]2)c1]

### 4-chloro-3-(5,7-difluoro-4-oxo-6-(1H-pyrazol-4-yl)-1,4-dihdroquinolin-2-yl)benzonitrile

### Step 1, 3,5-difluoro-4-(1-(tetrahydro-2H-pyrazol-4-yl)-pyrazol-4-yl)aniline

To a solution of 4-bromo-3,5-difluoroaniline (1 g, 4.8 mmol, 1.0 equiv.) and 1-(tetrahydro-2H-pyran-2-yl)-4-(4,4,5,5-tetramethyl-1,3,2-dioxaborolan-2-yl)-1H-pyrazole (2.0 g, 7.2 mmol, 1.5 equiv.) in dioxane (9 mL) and H₂O (3 mL) was added K₂CO₃ (2.0 g, 14.4 mmol, 3.0 equiv.) and Pd(dppf)Cl₂·CH₂Cl₂ (393 mg, 481 μmol, 0.1 equiv.) at 20° C. under N₂. The mixture was stirred at 90° C. for 16 hours under N₂. The reaction mixture was diluted with water (100 mL) and extracted with ethyl acetate (3×100 mL). The combined organic layers were washed with brine (50 mL), dried with anhydrous Na₂SO₄, filtered, and concentrated. The residue was purified by silica gel column chromatography (17% to 100% ethyl acetate in petroleum ether) to afford the title compound (1.3 g, 97% yield) as a yellow solid. ¹H NMR (400 MHz, CDCl₃) δ 7.99-7.92 (m, 2H), 6.33-6.24 (m, 2H), 5.42 (dd, J=2.5, 9.8 Hz, 1H), 4.09 (br dd, J=2.5, 10.4 Hz, 1H), 3.78-3.68 (m, 1H), 2.25-2.01 (m, 3H), 1.79-1.58 (m, 3H).

### Step 2, 2-bromo-3,5-difluoro-4-(1-(tetrahydro-2H-pyran-2-yl)-1H-pyrazol-4-yl)aniline

A solution of N-bromosuccinimide (382 mg, 2.2 mmol, 1.0 equiv.) in THF (3 mL) was added dropwise to a mixture of 3,5-difluoro-4-(1-(tetrahydro-2H-pyran-2-yl)-1H-pyrazol-4-yl)aniline (600 mg, 2.2 mmol, 1.0 equiv.) in THF (5 mL) at −10° C. under N₂. The mixture was stirred at −10-0° C. for 1 hour. The reaction mixture was diluted with water (25 mL) and extracted with ethyl acetate (3×30 mL). The combined organic layers were washed with brine (20 mL), dried with anhydrous Na₂SO₄, filtered, and concentrated. The residue was purified by silica gel column chromatography (25% to 100% ethyl acetate in petroleum ether) to afford the title compound (620 mg, 81% yield) as a yellow solid. ¹H NMR (400 MHz, CDCl₃) δ 7.96 (br d, J=16.2 Hz, 2H), 6.42 (dd, J=1.8, 12.2 Hz, 1H), 5.44 (dd, J=2.6, 9.4 Hz, 1H), 4.13-4.05 (m, 1H), 3.73 (dt, J=2.8, 11.2 Hz, 1H), 2.25-2.01 (m, 3H), 1.75-1.63 (m, 3H).

### Step 3, 2-(1-ethoxyvinyl)-3,5-difluoro-4-(1-(tetrahydro-2H-pyran-2-yl)-1H-pyrazol-4-yl)aniline

To a solution of 2-bromo-3,5-difluoro-4-(1-(tetrahydro-2H-pyran-2-yl)-1H-pyrazol-4-yl)aniline (470 mg, 1.3 mmol, 1.0 equiv.) and tributyl(1-ethoxyvinyl)stannane (531 μL, 1.6 mmol, 1.2 equiv.) in toluene (6 mL) was added Pd(PPh₃)₄ (152 mg, 131 μmol, 0.1 equiv.) at 20° C. under N₂. The mixture was stirred at 120° C. for 16 hours under N₂. The mixture was added to an aqueous solution of KF (30 mL) and then stirred for 1 hour. The mixture was diluted with water (20 mL) and extracted with ethyl acetate (3×30 mL). The combined organic layers were washed with brine (20 mL), dried over sodium sulfate, and concentrated under reduced pressure. The residue was purified by silica gel column chromatography (10% to 100% ethyl acetate in petroleum ether) to afford the title compound (270 mg, 59% yield) as a yellow oil. ¹H NMR (400 MHz, CDCl₃) δ 8.03-7.89 (m, 2H), 6.57 (d, J=11.4 Hz, 1H), 6.25 (dd, J=1.4, 12.6 Hz, 1H), 5.44 (ddd, J=3.0, 9.4, 12.6 Hz, 1H), 4.19-4.01 (m, 1H), 3.80-3.65 (m, 2H), 2.63 (d, J=9.0 Hz, 1H), 2.50 (s, 1H), 2.28-2.01 (m, 3H), 1.84-1.57 (m, 3H), 1.30-1.19 (m, 3H).

### Step 4, N-(2-acetyl-3,5-difluoro-4-(1H-pyrazol-4-yl)phenyl)-2-chloro-5-cyanobenzamide

To a solution of 2-(1-ethoxyvinyl)-3,5-difluoro-4-(1-(tetrahydro-2H-pyran-2-yl)-1H-pyrazol-4-yl)aniline (230 mg, 658 μmol, 1.0 equiv.) in THF (2 mL) was added NaH (26.3 mg, 658 μmol, 1.0 equiv.; 60% dispersion in ooil) at 0° C. The mixture was stirred at 0° C. for 5 minutes under N₂. Then 2-chloro-5-cyanobenzoyl chloride (158 mg, 800 μmol, 1.2 equiv.) in THF (1 mL) was added dropwise to the mixture at 0° C. and the mixture was stirred at 20° C. for 16 hours. The reaction mixture was quenched by the addition of aqueous HCl (2 M, 1 mL). Then HCl (12 M, 1 mL) was added to the mixture and the mixture was stirred at 20° C. for 2 hours. Then the mixture was diluted with NaHCO₃ (20 mL) and extracted with ethyl acetate (3×20 mL). The combined organic layers were washed with brine, dried over Na₂SO₄, filtered, and concentrated under reduced pressure. The crude product was triturated with ethyl acetate (5 mL) at 20° C. for 20 minutes. Then the residue was triturated with MeOH (5 mL) at 20° C. for 20 minutes to afford the title compound (100 mg, 38% yield) as a yellow solid. ¹H NMR (400 MHz, DMSO-d₆) δ 13.31 (br s, 1H), 11.01 (br s, 1H), 8.11 (d, J=1.8 Hz, 2H), 8.04 (dd, J=2.0, 8.4 Hz, 1H), 7.96-7.82 (m, 2H), 7.51 (d, J=11.6 Hz, 1H), 2.59 (d, J=3.8 Hz, 3H).

### Step 5, 4-chloro-3-(5,7-difluoro-4-oxo-6-(1H-pyrazol-4-yl)-1,4-dihydroquinolin-2-yl)benzamide

To a solution of N-(2-acetyl-3,5-difluoro-4-(1H-pyrazol-4-yl)phenyl)-2-chloro-5-cyanobenzamide (100 mg, 250 μmol, 1.0 equiv.) in dioxane (5 mL) was added NaOH (49.9 mg, 1.3 mmol, 5.0 equiv.) at 20° C. The mixture was stirred at 110° C. for 16 hours under N₂. The pH of the mixture was adjusted to 6 with aqueous HCl solution (1 M). Then the reaction mixture was diluted with water (15 mL) and extracted with ethyl acetate (3×15 mL). The combined organic layers were washed with brine, dried over Na₂SO₄, filtered, and concentrated under reduced pressure. Then the crude product was triturated with water (2 mL) at 20° C. for 20 minutes and filtered. The filtered cake was washed with water (3×1 mL). Then the filtered cake was concentrated to afford the title compound (80 mg, crude) as a yellow solid.

### Step 6, 4-chloro-3-(5,7-difluoro-4-oxo-6-(1H-pyrazol-4-yl)-1,4-dihydroquinolin-2-yl)benzonitrile

To a solution of 4-chloro-3-(5,7-difluoro-4-oxo-6-(1H-pyrazol-4-yl)-1,4-dihydroquinolin-2-yl)benzamide (80 mg, 200 μmol, 1.0 equiv.) in DCM (3 mL) was added triethylamine (139 μL, 998 μmol, 5 equiv.) and trifluoroacetic anhydride (69 μL, 499 μmol, 2.5 equiv.) at 20° C. The mixture was stirred at 20° C. for 1 hour under N₂. The pH of the mixture was adjusted to 6 with an aqueous solution of HCl (1 M). Then the reaction mixture was diluted with water (15 mL) and extracted with ethyl acetate (3×20 mL). The combined organic layers were washed with brine, dried over Na₂SO₄, filtered, and concentrated under reduced pressure. The residue was purified by preparative HPLC (column: Phenomenex Luna C18 75×30 mm×3 um; mobile phase: 10-50% acetonitrile in water (+0.2% formic acid)) to afford the title compound (13.1 mg, 17% yield) as a white solid. LCMS: [M+1]=383.0. ¹H NMR (400 MHz, DMSO-d₆) δ 13.27 (br s, 1H), 12.09 (br s, 1H), 8.22 (br d, J=19.8 Hz, 2H), 8.08 (br d, J=8.4 Hz, 1H), 7.93 (br d, J=8.4 Hz, 2H), 7.23 (br d, J=11.6 Hz, 1H), 6.07 (br s, 1H).

### Example 63

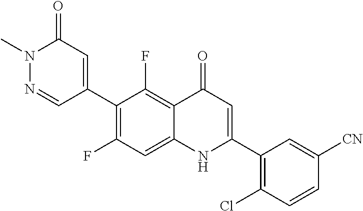 [SMILES: Cn1ncc(-c2c(F)cc3[nH]c(-c4cc(C#N)ccc4Cl)cc(=O)c3c2F)cc1=O]

### 4-chloro-3-(5,7-difluoro-6-(1-methyl-6-oxo-1,6-dihydropyridazin-4-yl)-4-oxo-1,4-dihydroquinolin-2-yl)benzonitrile

### Step 1, 5-(3-acetyl-4-amino-2,6-difluorophenyl)-2-methylpyridazin-3(2H)-one

To a solution of 5-iodo-2-methylpyridazin-3(2H)-one (2.1 g, 7.0 mmol, 1.5 equiv.), 1-(6-amino-2,4-difluoro-3-(4,4,5,5-tetramethyl-1,3,2-dioxaborolan-2-yl)phenyl)ethan-1-one (1.1 g, 4.7 mmol, 1.0 equiv.) and K₃PO₄ (2.2 g, 10.3 mmol, 2.2 equiv.) in water (25 mL) and THF (99 mL) was added dichloro[1,1′-bis(di-t-butylphosphino)ferrocene]palladium(II) (304 mg, 466 μmol, 0.1 equiv.) at 20° C. under N₂. The mixture was stirred at 80° C. for 2 hours. The reaction mixture was diluted with water (30 mL) and extracted with ethyl acetate (3×40 mL). The combined organic layers were dried over Na₂SO₄, filtered, and concentrated under reduced pressure to give a residue. The residue was purified by silica gel column chromatography (10% MeOH in DCM) to afford the title compound (850 mg, 65% yield) as a brown solid. LCMS [M+1]=280.1. ¹H NMR (400 MHz, DMSO-d₆) δ 7.98 (d, J=2.0 Hz, 1H), 7.83 (br s, 2H), 7.00 (s, 1H), 6.57 (dd, J=1.2, 12.8 Hz, 1H), 3.68 (s, 3H), 2.55-2.52 (m, 3H).

### Step 2, 5-(3-acetyl-2,6-difluoro-4-iodophenyl)-2-methylpyridazin-3(2H)-one

To a mixture of 5-(3-acetyl-4-amino-2,6-difluorophenyl)-2-methylpyridazin-3(2H)-one (734 mg, 2.6 mmol, 1.0 equiv.) and CuI (1 g, 5.3 mmol, 2.0 equiv.) in acetonitrile (8 mL) was added t-BuONO (949 mg, 9.2 mmol, 1.1 mL, 3.5 equiv.) at 20° C. under N₂. The mixture was stirred at 70° C. for 1 hour. The reaction mixture was diluted with acetonitrile (15 mL). The mixture was concentrated to give the crude product. The crude product was triturated with DCM and MeOH at 25° C. for 30 minutes. The filtrate was concentrated and the resulting residue was purified by silica gel column chromatography (50% ethyl acetate in petroleum ether) to afford the title compound (565 mg, 55% yield) as a white solid. ¹H NMR (400 MHz, CHLOROFORM-d) δ 7.80 (d, J=2.0 Hz, 1H), 7.62 (dd, J=1.6, 8.6 Hz, 1H), 7.06 (d, J=1.0 Hz, 1H), 3.85 (s, 3H), 2.60 (d, J=1.6 Hz, 3H).

### Step 3, N-(2-acetyl-3,5-difluoro-4-(1-methyl-6-oxo-1,6-dihydropyridazin-4-yl)phenyl)-2-chloro-5-cyanobenzamide

To a mixture of 5-(3-acetyl-2,6-difluoro-4-iodophenyl)-2-methylpyridazin-3(2H)-one (200 mg, 513 μmol, 1.0 equiv.), 2-chloro-5-cyanobenzamide (139 mg, 769 μmol, 1.5 equiv.), Cs₂CO₃ (251 mg, 769 μmol, 1.5 equiv.), and 4,5-bis(diphenylphosphino)-9,9-dimethylxanthene (44.5 mg, 76.9 μmol, 0.15 equiv.) in dioxane (6 mL) was added Pd(OAc)₂ (11.5 mg, 51.2 μmol, 0.1 equiv.) at 20° C. under N₂. The mixture was stirred at 45° C. for 2 hours. The reaction mixture was diluted with water (10 mL) and extracted with ethyl acetate (3×15 mL). A solid precipitated from this mixture and was filtered off to afford the title compound (102 mg, 45% yield) as a white solid. ¹H NMR (400 MHz, DMSO-d₆) δ 11.20 (s, 1H), 8.11 (dd, J=1.6, 9.0 Hz, 2H), 8.05 (dd, J=2.0, 8.4 Hz, 1H), 7.85 (d, J=8.4 Hz, 1H), 7.59 (d, J=11.2 Hz, 1H), 7.22 (s, 1H), 3.71 (s, 3H), 2.58 (d, J=3.8 Hz, 3H).

### Step 4, 4-chloro-3-(5,7-difluoro-6-(1-methyl-6-oxo-1,6-dihydropyridazin-4-yl)-4-oxo-1,4-dihydroquinolin-2-yl)benzonitrile

LiOH (5.4 mg, 226 μmol, 1.0 equiv.) was added to a solution of N-(2-acetyl-3,5-difluoro-4-(1-methyl-6-oxo-1,6-dihydropyridazin-4-yl)phenyl)-2-chloro-5-cyanobenzamide (100 mg, 226 μmol, 1.0 equiv.) in dioxane (5 mL) at 20° C. under N₂. The mixture was stirred at 110° C. for 30 hours. The pH was adjusted to 5 by the addition of an aqueous solution of HCl (1M). A solid precipitated out of the solution. The crude product was washed with water at 20° C. for 0.5 hours to afford the title compound (34.2 mg, 36% yield) as a white solid. LCMS [M+1]=425.1. ¹H NMR (400 MHz, DMSO-d₆) δ 12.42-12.17 (s, 1H), 8.25 (d, J=1.2 Hz, 1H), 8.14-8.01 (m, 2H), 7.93 (d, J=8.4 Hz, 1H), 7.29 (br d, J=9.2 Hz, 1H), 7.17 (s, 1H), 6.15 (br s, 1H), 3.72 (s, 3H).

### Example 64

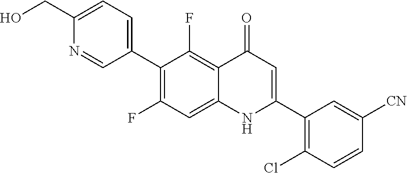 [SMILES: N#Cc1ccc(Cl)c(-c2cc(=O)c3c(F)c(-c4ccc(CO)nc4)c(F)cc3[nH]2)c1]

### 4-chloro-3-(5,7-difluoro-6-(6-(hydroxymethyl)pyridin-3-yl)-4-oxo-1,4-dihydroquinolin-2-yl)benzonitrile

### 1-(6-amino-2,4-difluoro-3-(4,4,5,5-tetramethyl-1,3,2-dioxaborolan-2-yl)phenyl)ethan-1-one

To a mixture of 1-(6-amino-3-bromo-2,4-difluoro-phenyl)ethanone (10 g, 40.0 mmol, 1.0 equiv.) and 4,4,5,5-tetramethyl-2-(4,4,5,5-tetramethyl-1,3,2-dioxaborolan-2-yl)-1,3,2-dioxaborolane (30.5 g, 120 mmol, 3.0 equiv.) in toluene (200 mL) was added KOAc (7.85 g, 80.0 mmol, 2.0 equiv.) and [2-(2-aminophenyl)phenyl]-chloro-palladium; dicyclohexyl-[3-(2,4,6-triisopropylphenyl)phenyl]phosphane (944.01 mg, 1.20 mmol, 0.03 equiv.) at 20° C. under N₂. The mixture was stirred at 80° C. for 5 hours. The reaction mixture was filtered and concentrated under reduced pressure. The crude product was purified by silica gel column chromatography (2% to 6% ethyl acetate in petroleum ether) to afford the title compound (5.5 g, 46% yield) as white solid. LCMS [M+1]=298.1. ¹H NMR (400 MHz, METHANOL-d₄) δ 6.21 (dd, J=1.2, 11.6 Hz, 1H), 2.53 (d, J=8.8 Hz, 3H), 1.33 (s, 12H).

### Scheme 5, step 1. 1-(6-amino-3-(6-(((tert-butyldimethylsilyl)oxy)methyl)pyridin-3-yl)-2,4-difluorophenyl)ethan-1-one

To a solution of 5-bromo-2-(((tert-butyldimethylsilyl)oxy)methyl)pyridine (508.0 mg, 1.7 mmol, 1.0 equiv.) and 1-(6-amino-2,4-difluoro-3-(4,4,5,5-tetramethyl-1,3,2-dioxaborolan-2-yl)phenyl)ethan-1-one (500 mg, 1.7 mmol, 1.0 equiv.) in dioxane (7.5 mL) and water (2.5 mL) was added K₂CO₃ (697 mg, 5.1 mmol, 3.0 equiv.) and then the mixture was degassed with N₂. Pd(dppf)Cl₂—CH₂Cl₂ (137 mg, 168 μmol, 0.1 equiv.) was added and the mixture was stirred at 80° C. for 16 hours under N₂. The reaction mixture was diluted with water (20 mL) and extracted with ethyl acetate (3×10 mL). The combined organic layers were washed with brine (10 mL), dried over Na₂SO₄, filtered, and concentrated under reduced pressure. The residue was purified by silica gel column chromatography (15% to 20% ethyl acetate in petroleum ether) to afford the title compound (350 mg, 53% yield) as a yellow solid. ¹H NMR (400 MHz, CHLOROFORM-d) δ 8.55 (s, 1H), 7.80 (br d, J=7.4 Hz, 1H), 7.68-7.61 (m, 1H), 6.60 (br s, 2H), 6.28 (dd, J=1.4, 11.4 Hz, 1H), 4.93 (s, 2H), 2.61 (d, J=9.0 Hz, 3H), 1.00-0.98 (m, 11H), 0.17-0.15 (m, 6H).

### Scheme 5, step 2. N-(2-acetyl-4-(6-(((tert-butyldimethylsilyl)oxy)methyl)pyridin-3-yl)-3,5-difluorophenyl)-2-chloro-N-(2-chloro-5-cyanobenzoyl)-5-cyanobenzamide

To a solution of 1-(6-amino-3-(6-(((tert-butyldimethylsilyl)oxy)methyl)pyridin-3-yl)-2,4-difluorophenyl)ethan-1-one (250 mg, 637 μmol, 1.0 equiv.) in THF (4.0 mL) was added NaH (127 mg, 3.2 mmol, 5.0 equiv.; 60% dispersion in oil) at 0° C. Then 2-chloro-5-cyanobenzoyl chloride (637 mg, 3.2 mmol, 5.0 equiv.) was added and the solution was degassed N₂ and stirred at 15° C. for 16 hours under N₂. The reaction mixture was quenched with water (30 mL) and extracted with ethyl acetate (3×10 mL). The combined organic layers were washed with brine (10 mL), filtered, and concentrated under reduced pressure. The residue was purified by silica gel column chromatography (12% to 18% ethyl acetate in petroleum ether) to afford the title compound (310 mg, crude) as a yellow solid. ¹H NMR (400 MHz, CHLOROFORM-d) δ 8.64 (s, 1H), 7.98 (d, J=1.7 Hz, 2H), 7.75-7.71 (m, 2H), 7.65-7.63 (m, 1H), 7.62-7.60 (m, 1H), 7.52 (d, J=8.5 Hz, 2H), 7.16 (dd, J=1.4, 8.9 Hz, 1H), 4.95 (s, 2H), 2.71 (d, J=4.2 Hz, 3H), 0.99 (br s, 9H), 0.17 (s, 6H).

### N-(2-acetyl-4-(6-(((tert-butyldimethylsilyl)oxy)methyl)pyridin-3-yl)-3,5-difluorophenyl)-2-chloro-5-cyanobenzamide

In certain instances, such as the one described immediately above, when the dibenzoylated intermediate is obtained it is necessary to remove one acyl group before cyclization to the quinolone. Dibenzoylation does not always occur, and the step described here is not necessary in those instances.

To a solution of N-(2-acetyl-4-(6-(((tert-butyldimethylsilyl)oxy)methyl)pyridin-3-yl)-3,5-difluorophenyl)-2-chloro-N-(2-chloro-5-cyanobenzoyl)-5-cyanobenzamide (301 mg, 418 μmol, 1.0 equiv.) in isopropanol (5.0 mL) was added K₂CO₃ (116 mg, 836 μmol, 2.0 equiv.). The reaction mixture was stirred at 50° C. under N₂ for 1 hour. The reaction was diluted with water (30 mL) and extracted with ethyl acetate (3×10 mL). The combined organic layers were washed with brine (10 mL) and concentrated under reduced pressure. The residue was purified by preparative TLC (25% ethyl acetate in petroleum ether) to afford the title compound (125 mg, 54% yield) as a yellow solid. ¹H NMR (400 MHz, CHLOROFORM-d) δ 12.22 (s, 1H), 8.68-8.60 (m, 2H), 7.95 (d, J=1.8 Hz, 1H), 7.85 (br d, J=7.4 Hz, 1H), 7.76-7.72 (m, 1H), 7.70 (br d, J=8.0 Hz, 1H), 7.66 (s, 1H), 4.94 (br s, 2H), 2.71 (d, J=8.8 Hz, 3H), 1.00 (s, 9H), 0.17 (s, 6H).

### Scheme 5, step 3. 3-(6-(6-(((tert-butyldimethylsilyl)oxy)methyl)pyridin-3-yl)-57-difluoro-4-oxo-1,4-dihydroquinolin-2-yl)-4-chlorobenzonitrile

To a solution of N-(2-acetyl-4-(6-(((tert-butyldimethylsilyl)oxy)methyl)pyridin-3-yl)-3,5-difluorophenyl)-2-chloro-5-cyanobenzamide (150 mg, 270 μmol, 1.0 equiv.) in dioxane (2.0 mL) was added NaOH (11.0 mg, 270 μmol, 1.0 equiv.). The mixture was stirred for 1.5 hours. The reaction mixture was quenched with an aqueous solution of HCl (1 M) at 15° C., and then diluted with water (20 mL) and extracted with ethyl acetate (3×10 mL). The combined organic layers were washed with brine (10 mL) and concentrated under reduced pressure to give a residue. The residue was purified by preparative TLC (25% ethyl acetate in petroleum ether) to afford the title compound (65.0 mg, 45% yield) as a yellow solid.

### 4-chloro-3-(5,7-difluoro-6-(6-(hydroxymethyl)pyridin-3-yl)-4-oxo-1,4-dihydroquinolin-2-yl)benzonitrile

A mixture of 3-(6-(6-(((tert-butyldimethylsilyl)oxy)methyl)pyridin-3-yl)-5,7-difluoro-4-oxo-1,4-dihydroquinolin-2-yl)-4-chlorobenzonitrile (65.0 mg, 121 μmol, 1.0 equiv.) in HCl/dioxane (2.0 mL) was degassed with N₂, and the mixture was stirred at 15° C. for 1 hour under N₂. The reaction mixture was concentrated and the residue was purified by preparative HPLC (column: Phenomenex Luna 80×30 mm×3 um; mobile phase: 10-40% acetonitrile in water (+0.04% HCl)) to afford the title compound (14.2 mg, 28% yield) as a white solid. LCMS: [M+1]=424.0. ¹H NMR (400 MHz, DMSO-d₆) δ 12.74-11.84 (m, 1H), 8.77 (s, 1H), 8.31-8.19 (m, 2H), 8.10 (dd, J=2.0, 8.4 Hz, 1H), 7.94 (d, J=8.2 Hz, 1H), 7.83 (d, J=8.2 Hz, 1H), 7.36 (br d, J=10.4 Hz, 1H), 6.19 (br s, 1H), 4.76 (s, 2H).

### Example 65

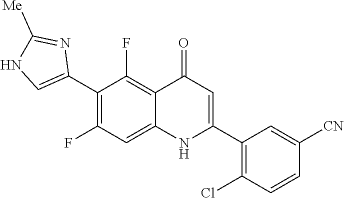 [SMILES: Cc1nc(-c2c(F)cc3[nH]c(-c4cc(C#N)ccc4Cl)cc(=O)c3c2F)c[nH]1]

### 4-chloro-3-(5,7-difluoro-6-(2-methyl-1H-imidazol-4-yl)-4-oxo-1,4-dihydroquinolin-2-yl)benzonitrile

The precursor to the title compound, 4-chloro-3-(5,7-difluoro-6-(2-methyl-1-((2-(trimethylsilyl)ethoxy)methyl)-1H-imidazol-4-yl)-4-oxo-1,4-dihydroquinolin-2-yl)benzonitrile, was prepared following Scheme 5 in a manner similar to that described for Example 64 using 4-bromo-2-methyl-1-((2-(trimethylsilyl)ethoxy)methyl)-1H-imidazole as the starting material.

### 4-chloro-3-(5,7-difluoro-6-(2-methyl-1H-imidazol-4-yl)-4-oxo-1,4-dihydroquinolin-2-yl)benzonitrile

A solution of 4-chloro-3-(5,7-difluoro-6-(2-methyl-1-((2-(trimethylsilyl)ethoxy)methyl)-1H-imidazol-4-yl)-4-oxo-1,4-dihydroquinolin-2-yl)benzonitrile (60 mg, 114 μmol, 1.0 equiv.) in TFA (0.5 mL) and DCM (0.5 mL) was stirred at 20° C. for 1 hour. The reaction mixture was concentrated under reduced pressure and the residue was purified by preparative HPLC (column: Waters Xbridge BEH C18 100×30 mm×10 um; mobile phase: 5-35% acetonitrile in water (+NH₄HCO₃)) to afford the title compound (10.3 mg, 22% yield) as a yellow solid. LCMS [M+1]=397.0. ¹H NMR (400 MHz, METHANOL-d₄) δ 8.07 (d, J=2.0 Hz, 1H), 7.94 (dd, J=2.0, 8.3 Hz, 1H), 7.87-7.80 (m, 1H), 7.35 (s, 1H), 7.26-7.17 (m, 1H), 6.29 (s, 1H), 2.47 (s, 3H).

### Example 66

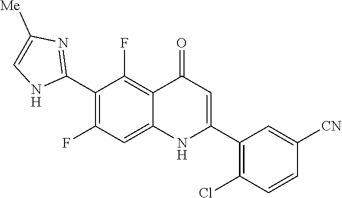 [SMILES: Cc1c[nH]c(-c2c(F)cc3[nH]c(-c4cc(C#N)ccc4Cl)cc(=O)c3c2F)n1]

### 4-chloro-3-(5,7-difluoro-6-(2-methyl-1H-imidazol-4-yl)-4-oxo-1,4-dihydroquinolin-2-yl)benzonitrile

The precursor to the title compound, 4-chloro-3-(5,7-difluoro-6-(4-methyl-1-((2-(trimethylsilyl)ethoxy)methyl)-1H-imidazol-2-yl)-4-oxo-1,4-dihydroquinolin-2-yl)benzonitrile, was prepared following Scheme 5 in a manner similar to that described for Example 64 using 2-bromo-4-methyl-1-((2-(trimethylsilyl)ethoxy)methyl)-1H-imidazole as the starting material.

### 4-chloro-3-(5,7-difluoro-6-(2-methyl-1H-imidazol-4-yl)-4-oxo-1,4-dihydroquinolin-2-yl)benzonitrile

This compound was prepared in a similar manner to that described for Example 65. LCMS [M+1]=397.0. ¹H NMR (400 MHz, METHANOL-d₄) δ 8.09 (d, J=1.8 Hz, 1H), 7.97 (dd, J=2.0, 8.4 Hz, 1H), 7.86 (d, J=8.4 Hz, 1H), 7.52 (d, J=0.9 Hz, 1H), 7.41 (dd, J=1.6, 11.2 Hz, 1H), 6.41 (s, 1H), 2.48 (d, J=0.6 Hz, 3H).

### Example 67

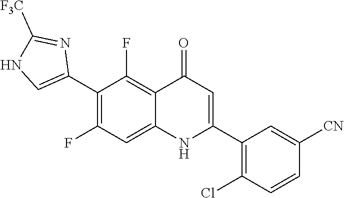 [SMILES: N#Cc1ccc(Cl)c(-c2cc(=O)c3c(F)c(-c4c[nH]c(C(F)(F)F)n4)c(F)cc3[nH]2)c1]

### 4-chloro-3-(5,7-difluoro-4-oxo-6-(2-(trifluoromethyl)-1H-imidazol-4-yl)-1,4-dihydroquinolin-2-yl)benzonitrile

The precursor to the title compound, 4-chloro-3-(5,7-difluoro-4-oxo-6-(2-(trifluoromethyl)-1-((2-(trimethylsilyl)ethoxy)methyl)-1H-imidazol-4-yl)-1,4-dihydroquinolin-2-yl)benzonitrile, was prepared following Scheme 5 in a manner similar to that described for Example 64 using 4-bromo-2-(trifluoromethyl)-1-((2-(trimethylsilyl)ethoxy)methyl)-1H-imidazole as the starting material.

### 4-chloro-3-(5,7-difluoro-4-oxo-6-(2-(trifluoromethyl)-1H-imidazol-4-yl)-1,4-dihydroquinolin-2-yl)benzonitrile

This compound was prepared in a similar manner to that described for Example 65. LCMS [M+1]=450.9. ¹H NMR (400 MHz, DMSO-d₆) δ 13.97-13.77 (m, 1H), 12.24-12.10 (m, 1H), 8.25 (d, J=1.8 Hz, 1H), 8.08 (dd, J=2.0, 8.4 Hz, 1H), 7.93 (d, J=8.5 Hz, 1H), 7.86-7.63 (m, 1H), 7.24 (br d, J=9.0 Hz, 1H), 6.26-6.01 (m, 1H).

### Example 68

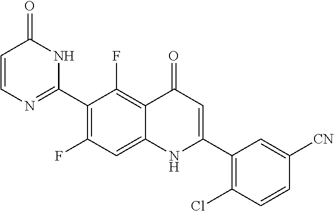 [SMILES: N#Cc1ccc(Cl)c(-c2cc(=O)c3c(F)c(-c4nccc(=O)[nH]4)c(F)cc3[nH]2)c1]

### 4-chloro-3-(5,7-difluoro-4-oxo-6-(6-oxo-1,6-dihydropyrimidin-2-yl)-1,4-dihydroquinolin-2-yl)benzonitrile

The precursor to the title compound, 4-chloro-3-(5,7-difluoro-6-(4-((4-methoxybenzyl)oxy)pyrimidin-2-yl)-4-oxo-1,4-dihydroquinolin-2-yl)benzonitrile, was prepared following Scheme 5 in a similar manner to that described for Example 64 using 2-chloro-4-((4-methoxybenzyl)oxy)pyrimidine as the starting material.

### 4-chloro-3-(5,7-difluoro-4-oxo-6-(6-oxo-1,6-dihydropyrimidin-2-yl)_{1,4} dihdroquinolin-2-yl)benzonitrile

This compound was prepared in a similar manner to that described for Example 65. LCMS [M+1]=411.0. ¹H NMR (400 MHz, DMSO-d₆) δ 13.46-12.96 (m, 1H), 12.33 (br s, 1H), 8.29 (s, 1H), 8.16-8.04 (m, 2H), 7.95 (d, J=8.4 Hz, 1H), 7.26 (br d, J=10.2 Hz, 1H), 6.46 (br s, 1H), 6.16 (s, 1H).

The compounds in Table 5 were prepared following Scheme 5 using similar procedures as those described for Example 64. Protection of the aryl halide is not always required. In these instances, the final step can be omitted.

TABLE 5

| | | IUPAC | | LCMS |
|---|---|---|---|---|
| Example number | Structure | Name | ¹H NMR | Exact Mass |
| 69 | 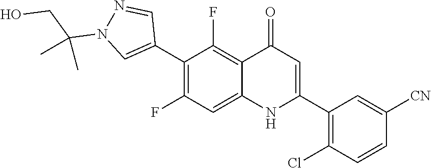 [SMILES: CC(C)(CO)n1cc(-c2c(F)cc3[nH]c(-c4cc(C#N)ccc4Cl)cc(=O)c3c2F)cn1] | 4-chloro-3- (5,7-difluoro- 6-(1-(1- hydroxy-2- methylpropan- 2-yl)-1H- pyrazol-4-yl)- 4-oxo-1,4- dihydroquinolin- 2-yl)benzonitrile | (400 MHz, METHANOL- d4) δ = 8.32 (br s, 1H), 8.17 (d, J = 1.8 Hz, 1H), 8.08 (br s, 1H), 8.02 (dd, J = 1.6, 8.4 Hz, 1H), 7.91 (d, J = 8.6 Hz, 1H), 7.53 (br d, J = 11.2 Hz, 1H), | Calculated m/z 455.1, found m/z 455.0 |
| | | | 6.92 (s, 1H), 3.81 | |
| | | | (s, 2H), 1.64 (s, | |
| | | | 6H) | |
| 70 | 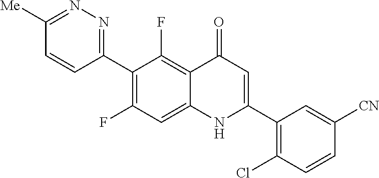 [SMILES: Cc1ccc(-c2c(F)cc3[nH]c(-c4cc(C#N)ccc4Cl)cc(=O)c3c2F)nn1] | 4-chloro-3- (5,7-difluoro- 6-(6- methylpyridazin- 3-yl)-4-oxo- 1,4- dihydroquinolin- 2-yl)benzonitrile | (400 MHz, DMSO- d₆) δ 12.57-12.04 (m, 1H), 8.27 (d, J = 1.6 Hz, 1H), 8.09 (dd, J = 1.8, 8.4 Hz, 1H), 7.94 (d, J = 8.2 Hz, 1H), 7.87 (d, J = 8.6 Hz, 1H), 7.79- 7.72 (m, 1H), 7.31 (br d, J = 10.2 Hz, 1H), 6.17 (br s, 1H), 2.73- 2.70 (m, 3H) | Calculated m/z 409.1, found m/z 409.0 |
| 71 | 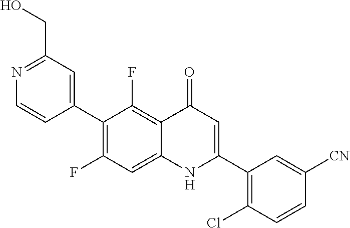 [SMILES: N#Cc1ccc(Cl)c(-c2cc(=O)c3c(F)c(-c4ccnc(CO)c4)c(F)cc3[nH]2)c1] | 4-chloro-3- (5,7-difluoro- 6-(2- (hydroxymethyl) pyridin-4-yl)- 4-oxo-1,4- dihydroquinolin- 2- yl)benzonitrile | (400 MHz, METHANOL-d₄) δ 8.78 (d, J = 5.8 Hz, 1H), 8.16-8.04 (m, 2H), 8.01-7.92 (m, 2H), 7.85 (d, J = 8.2 Hz, 1H), 7.33 (d, J = 10.8 Hz, 1H), 6.34 (s, 1H), 5.04-4.97 (m, 2H) | Calculated m/z 424.1, found m/z 424.0 |
| 72 | 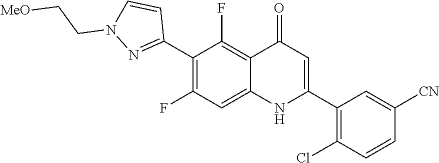 [SMILES: COCCn1ccc(-c2c(F)cc3[nH]c(-c4cc(C#N)ccc4Cl)cc(=O)c3c2F)n1] | 4-chloro-3- (5,7-difluoro- 6-(1-(2- methoxyethyl)- 1H-pyrazol-3- yl)-4-oxo-1,4- dihydroquinolin- 2-yl)benzonitrile | (400 MHz, DMSO- d₆) δ 12.37-12.02 (m, 1H), 8.25 (d, J = 2.0 Hz, 1H), 8.08 (dd, J = 2.1, 8.4 Hz, 1H), 7.93 (d, J = 8.4 Hz, 1H), 7.86 (d, J = 2.2 Hz, 1H), 7.22 (br d, J = 10.8 Hz, 1H), 6.54 (s, 1H), 6.13 (br s, 1H), 4.35 (t, J = 5.3 | Calculated m/z 441.1, found m/z 441.0 |
| | | | Hz, 2H), 3.74 (t, J = | |
| | | | 5.3 Hz, 2H), 3.25 | |
| | | | (s, 3H) | |
| 73 | 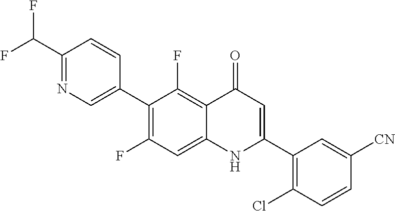 [SMILES: N#Cc1ccc(Cl)c(-c2cc(=O)c3c(F)c(-c4ccc(C(F)F)nc4)c(F)cc3[nH]2)c1] | 4-chloro-3-(6- (6- (difluoromethyl) pyridin-3-yl)- 5,7-difluoro-4- oxo-1,4- dihydroquinolin- 2-yl)benzonitrile | ¹H NMR (400 MHz, DMSO-d₆) δ 11.37 (br s, 1H), 7.94 (s, 1H), 7.37 (d, J = 2.0 Hz, 1H), 7.30 (br d, J = 8.8 Hz, 1H), 7.20 (dd, J = 2.0, 8.4 Hz, 1H), 7.07-6.96 (m, 3H), 6.43 (m, 1H), 6.30-6.02 (m, 1H), 5.26 (br s, 1H) | Calculated m/z 444.0, found m/z 444.0 |

### Example 74

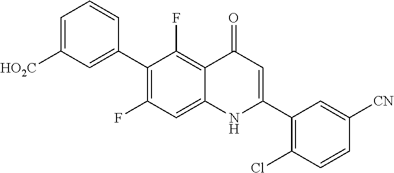 [SMILES: N#Cc1ccc(Cl)c(-c2cc(=O)c3c(F)c(-c4cccc(C(=O)O)c4)c(F)cc3[nH]2)c1]

### 3-(2-(2-chloro-5-cyanophenyl)-5,7-difluoro-4-oxo-1,4-dihydroquinolin-6-yl)benzoic acid

The precursor to the title compound, tert-butyl 3-(2-(2-chloro-5-cyanophenyl)-5,7-difluoro-4-oxo-1,4-dihydroquinolin-6-yl)benzoate, was prepared following Scheme 4.

3-(2-(2-chloro-5-cyanophenyl)-5,7-difluoro-4-oxo-1,4-dihydroquinolin-6-yl)benzoic acid: A mixture of tert-butyl 3-(2-(2-chloro-5-cyanophenyl)-5,7-difluoro-4-oxo-1,4-dihydroquinolin-6-yl)benzoate (70 mg, 142 μmol, 1.0 equiv.) in HCl/dioxane (3 mL) was stirred at 20° C. for 2 hours. The reaction was concentrated and the resulting residue was purified by preparative HPLC (column: Phenomenex Luna C18 75×30 mm×3 um; mobile phase: 40-70% acetonitrile in water (+0.2% formic acid)) to afford the title compound (19.6 mg, 30% yield) as a white solid. LCMS [M+1]=437.0. ¹H NMR (400 MHz, DMSO-d₆) δ 13.08-13.28 (br s, 1H), 12.14-12.27 (br s, 1H), 8.23-8.32 (s, 1H), 8.01-8.14 (m, 3H), 7.91-7.98 (m, 1H), 7.72-7.80 (m, 1H), 7.64-7.70 (m, 1H), 7.22-7.32 (d, J=10.4, 1H), 6.11 (br s, 1H).

The compounds in Table 6 were prepared following Scheme 4 using similar procedures to those described for Example 74.

TABLE 6

| | | | | LCMS |
|---|---|---|---|---|
| Example | | IUPAC | | Exact |
| number | Structure | Name | ¹H NMR | Mass |
| 75 | 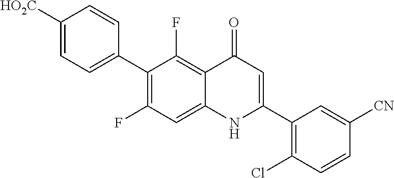 [SMILES: N#Cc1ccc(Cl)c(-c2cc(=O)c3c(F)c(-c4ccc(C(=O)O)cc4)c(F)cc3[nH]2)c1] | 4-(2-(2-chloro- 5-cyanophenyl)- 5,7-difluoro-4- oxo-1,4- dihydroquinolin- 6-yl)benzoic acid | (400 MHz, DMSO-d₆) δ ppm 13.10-13.16 (m, 1 H) 12.19-12.25 (m, 1 H) 8.24- 8.29 (m, 1 H) 8.08 (d, J = 8.4 Hz, 3 H) 7.92-7.97 (m, 1 H) 7.63 (br d, J = 8.2 Hz, 2 H) 7.24- 7.29 (m, 1 H) 6.10- 6.13 (m, 1 H) | Calculated m/z 437.0, found m/z 437.0 |

### Example 76

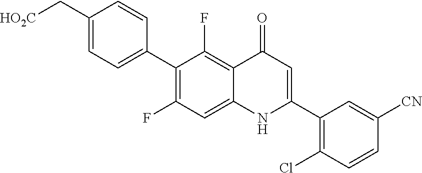 [SMILES: N#Cc1ccc(Cl)c(-c2cc(=O)c3c(F)c(-c4ccc(CC(=O)O)cc4)c(F)cc3[nH]2)c1]

### 2-(4-(2-(2-chloro-5-cyanophenyl)-5,7-difluoro-4-oxo-1,4-dihydroquinolin-6-yl)phenyl)acetic acid

The precursor to the title compound, methyl 2-(4-(2-(2-chloro-5-cyanophenyl)-5,7-difluoro-4-oxo-1,4-dihydroquinolin-6-yl)phenyl)acetate, was prepared following Scheme 4.

2-(4-(2-(2-chloro-5-cyanophenyl)-5,7-difluoro-4-oxo-1,4-dihydroquinolin-6-yl)phenyl)acetic acid: To a solution of methyl 2-(4-(2-(2-chloro-5-cyanophenyl)-5,7-difluoro-4-oxo-1,4-dihydroquinolin-6-yl)phenyl)acetate (30 mg, 64.5 μmol, 1.0 equiv.) in DCE (0.5 mL), was added Me₃SnOH (117 mg, 645 μmol, 10 equiv.). The mixture was stirred at 50° C. for 12 hours. The reaction mixture was quenched with HCl (1 M, 2 mL) and the mixture was extracted with DCM (2×3 mL). The combined organic layers were dried over Na₂SO₄, filtered, and concentrated under reduced pressure. The residue was purified by preparative HPLC (column: Phenomenex Luna C18 80×40 mm×3 um; mobile phase: 26-54% acetonitrile in water (+0.04% HCl)) to afford the title compound (10 mg, 34% yield) as a yellow solid. LCMS [M+1]=450.9. ¹H NMR (400 MHz, DMSO-d₆) δ 8.25 (d, J=2.0 Hz, 1H), 8.09 (dd, J=2.0, 8.4 Hz, 1H), 7.93 (d, J=8.4 Hz, 1H), 7.46-7.37 (m, 4H), 7.29 (br d, J=10.4 Hz, 1H), 6.16 (s, 1H), 3.66 (s, 2H).

### Example 77

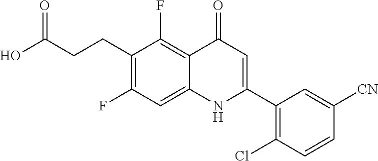 [SMILES: N#Cc1ccc(Cl)c(-c2cc(=O)c3c(F)c(CCC(=O)O)c(F)cc3[nH]2)c1]

### 3-(2-(2-chloro-5-cyanophenyl)-5,7-difluoro-4-oxo-1,4-dihydroquinolin-6-yl)propanoic acid

The precursor to the title compound, ethyl 3-(2-(2-chloro-5-cyanophenyl)-5,7-difluoro-4-oxo-1,4-dihydroquinolin-6-yl)propanoate, was prepared following Scheme 4.

### 3-(2-(2-chloro-5-cyanophenyl)-5,7-difluoro-4-oxo-1,4-dihydroquinolin-6-yl)propanoic acid

To a solution of ethyl 3-(2-(2-chloro-5-cyanophenyl)-5,7-difluoro-4-oxo-1,4-dihydroquinolin-6-yl)propanoate (55 mg, 132 μmol, 1.0 equiv.) in THF (4 mL) and water (2 mL) was added LiOH·H₂O (27.6 mg, 660 μmol, 5.0 equiv.). The mixture was stirred at 20° C. for 16 hours. The pH of the reaction mixture was adjusted to 3 with aqueous HCl (1 M). Then water (30 mL) was added into the mixture and yellow solid formed which was collected by filtration. The solid was purified by preparative HPLC (Column: Phenomenex Luna C18 150×30 mm×5 um; mobile phase: 20-55% acetonitrile in water (+0.2% formic acid)) to afford the title compound (15.8 mg, 30% yield) as a white solid. LCMS [M+1]=389.0. ¹H NMR (400 MHz, METHANOL-d₄) 6=8.05 (d, J=1.8 Hz, 1H), 7.97-7.89 (m, 1H), 7.83 (d, J=8.4 Hz, 1H), 7.11 (br d, J=9.8 Hz, 1H), 6.24 (s, 1H), 3.08 (br t, J=7.6 Hz, 2H), 2.63 (t, J=7.8 Hz, 2H).

### Example 78

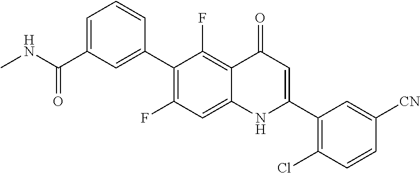 [SMILES: CNC(=O)c1cccc(-c2c(F)cc3[nH]c(-c4cc(C#N)ccc4Cl)cc(=O)c3c2F)c1]

3-(2-(2-chloro-5-cyanophenyl)-5,7-difluoro-4-oxo-1,4-dihydroquinolin-6-yl)-N-methylbenzamide: To a solution of 3-(2-(2-chloro-5-cyanophenyl)-5,7-difluoro-4-oxo-1,4-dihydroquinolin-6-yl)benzoic acid (60 mg, 137 μmol, 1.0 equiv.) in DMF (1 mL) was added HATU (57.4 mg, 151 μmol, 1.1 equiv.), DIPEA (119.6 uL, 687 μmol, 5.0 equiv.). Then methylamine-hydrochloride was added (12 mg, 178 μmol, 1.3 equiv.). The mixture was stirred at 20° C. for 16 hours. The reaction was diluted with water (10 mL) and extracted with ethyl acetate (3×15 mL). The combined organic layers were washed with brine (15 mL), dried over anhydrous Na₂SO₄, filtered and concentrated under reduced pressure. The residue was purified by preparative HPLC (column: Phenomenex Luna C18 75×30 mm×3 um; mobile phase: 20-50% acetonitrile in water (+0.2% formic acid)) to afford the title compound (11.5 mg, 18% yield) as a white solid. LCMS [M+1]=450.0. ¹H NMR (400 MHz, DMSO-d₆) δ 12.22 (br s, 1H), 8.55 (q, J=4.1 Hz, 1H), 8.26 (d, J=1.9 Hz, 1H), 8.09 (dd, J=8.4, 2.0 Hz, 1H), 7.85-7.99 (m, 3H), 7.54-7.69 (m, 2H), 7.28 (br d, J=10.2 Hz, 1H), 6.15 (br s, 1H), 2.80 (d, J=4.6 Hz, 3H).

The compounds in Table 7 were prepared using similar procedure described for Example 78. The acid precursors were made using the procedures described for Example 74 following Scheme 4. Additional information on the synthesis of the acid precursors can be found in the starting materials section.

TABLE 7

| | | | | LCMS |
|---|---|---|---|---|
| Example | | IUPAC | | Exact |
| number | Structure | Name | ¹H NMR | Mass |
| 79 | 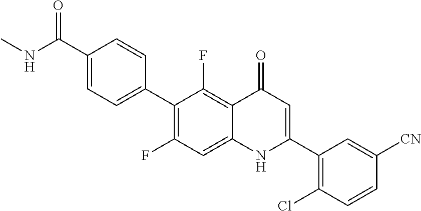 [SMILES: CNC(=O)c1ccc(-c2c(F)cc3[nH]c(-c4cc(C#N)ccc4Cl)cc(=O)c3c2F)cc1] | 4-(2-(2-chloro- 5-cyanophenyl)- 5,7-difluoro-4- oxo-1,4- dihydroquinolin- 6-yl)-N- methylbenzamide | (400 MHz, METHANOL-d₄) δ 8.10 (d, J = 1.8 Hz, 1H), 7.95 (d, J = 8.6 Hz, 3H), 7.87 (s, 1H), 7.62 (d, J = 8.2 Hz, 2H), 7.27 (dd, J = 10.2, 1.50 Hz, 1H), 6.37 (s, 1H), 2.84-3.08 (m, 3H) | Calculated m/z 450.1, found m/z 450.0 |
| 80 | 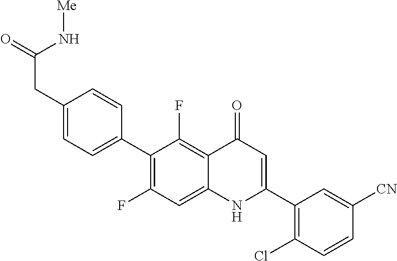 [SMILES: CNC(=O)Cc1ccc(-c2c(F)cc3[nH]c(-c4cc(C#N)ccc4Cl)cc(=O)c3c2F)cc1] | 2-(4-(2-(2- chloro-5- cyanophenyl)- 5,7-difluoro-4- oxo-1,4- dihydroquinolin- 6-yl)phenyl)-N- methylacetamide | (400 MHz, DMSO-d₆) δ 12.44- 11.93 (m, 1H), 8.26 (d, J = 1.8 Hz, 1H), 8.14- 8.02 (m, 2H), 7.94 (d, J = 8.2 Hz, 1H), 7.45-7.36 (m, 4H), 7.26 (br d, J = 9.8 Hz, 1H), 6.13 (br d, J = 4.2 Hz, 1H), 3.48 (s, 2H), 2.62-2.60 (m, 3H) | Calculated m/z 464.1, found m/z 464.4 |
| 81 | 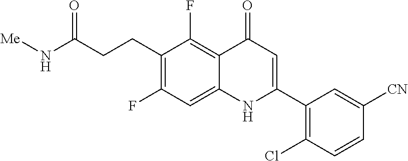 [SMILES: CNC(=O)CCc1c(F)cc2[nH]c(-c3cc(C#N)ccc3Cl)cc(=O)c2c1F] | 3-(2-(2-chloro- 5-cyanophenyl)- 5,7-difluoro-4- oxo-1,4- dihydroquinolin- 6-yl)-N- methylpropanamide | (400 MHz, DMSO-d₆) δ 12.24- 11.85 (m, 1H), 8.23 (d, J = 1.7 Hz, 1H), 8.08 (dd, J = 2.0, 8.4 Hz, 1H), 7.93 (d, J = 8.4 Hz, 1H), 7.82 (br d, J = 4.6 Hz, 1H), 7.13 (br d, J = 9.2 Hz, 1H), 6.27-5.88 (m, 1H), 2.89 (br t, J = 7.5 Hz, 2H), 2.56 (d, J = 4.5 Hz, 3H), 2.35 (br t, J = 7.6 Hz, 2H) | Calculated m/z 402.1, found m/z 402.0 |
| 82 | 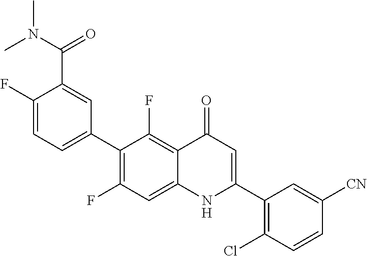 [SMILES: CN(C)C(=O)c1cc(-c2c(F)cc3[nH]c(-c4cc(C#N)ccc4Cl)cc(=O)c3c2F)ccc1F] | 5-(2-(2-chloro- 5-cyanophenyl)- 5,7-difluoro-4- oxo-1,4- dihydroquinolin- 6-yl)-2-fluoro- N,N- dimethylbenzamide | (400 MHz, DMSO-d₆) δ = 12.21 (br s, 1H), 8.25 (d, J = 1.6 Hz, 1H), 8.09 (dd, J = 1.8, 8.4 Hz, 1H), 7.49 (d, J = 8.4 Hz, 1H), 7.67- 7.58 (m, 1H), 7.53 (br d, J = 5.8 Hz, 1H), 7.47 (t, J = 9.0 Hz, 1H), 7.34- 7.16 (m, 1H), 6.11 (br s, 1H), 3.02 (s, 3H), 2.90 (s, 3H) | Calculated m/z 482.1, found m/z 482.0 |
| 83 | 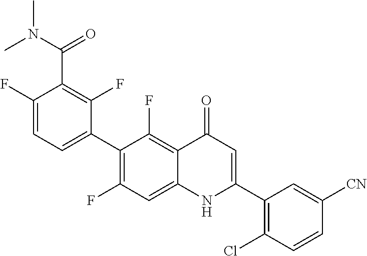 [SMILES: CN(C)C(=O)c1c(F)ccc(-c2c(F)cc3[nH]c(-c4cc(C#N)ccc4Cl)cc(=O)c3c2F)c1F] | 3-(2-(2-chloro- 5-cyanophenyl)- 5,7-difluoro-4- oxo-1,4- dihydroquinolin- 6-yl)-2,6- difluoro-N,N- dimethylbenzamide | (400 MHz, DMSO-d₆) δ 12.42-12.04 (m, 1H), 8.26 (d, J = 1.8 Hz, 1H), 8.10 (dd, J = 2.0, 8.2 Hz, 1H), 7.95 (d, J = 8.4 Hz, 1H), 7.73 (q, J = 7.2 Hz, 1H), 7.43 (t, J = 8.4 Hz, 1H), 7.31 (br d, J = 8.6 Hz, 1H), 6.23-6.10 (m, 1H), 3.10- 3.04 (m, 3H), 2.93 (d, J = 6.0 Hz, 3H) | Calculated m/z 500.1, found m/z 500.1 |
| 84 | 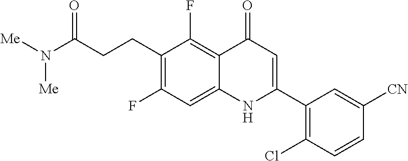 [SMILES: CN(C)C(=O)CCc1c(F)cc2[nH]c(-c3cc(C#N)ccc3Cl)cc(=O)c2c1F] | 3-(2-(2-chloro- 5-cyanophenyl)- 5,7-difluoro-4- oxo-1,4- dihydroquinolin- 6-yl)-N,N- dimethyl- propanamide | (400 MHz, DMSO-d₆) δ 12.24-11.88 (m, 1H), 8.23 (d, J = 1.8 Hz, 1H), 8.09 (dd, J = 2.0, 8.4 Hz, 1H), 7.93 (d, J = 8.4 Hz, 1H), 7.14 (br d, J = 9.6 Hz, 1H), 6.23-5.92 (m, 1H), 2.95 (s, 3H), 2.91-2.85 (m, 2H), 2.83 (s, 3H), 2.62-2.56 (m, 2H) | Calculated m/z 416.1, found m/z 416.0 |
| 85 | 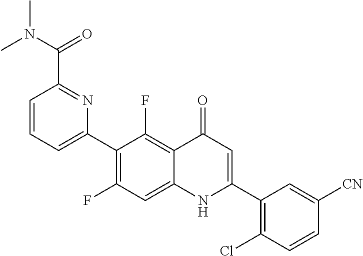 [SMILES: CN(C)C(=O)c1cccc(-c2c(F)cc3[nH]c(-c4cc(C#N)ccc4Cl)cc(=O)c3c2F)n1] | 6-(2-(2-chloro- 5-cyanophenyl)- 5,7-difluoro-4- oxo-1,4- dihydroquinolin- 6-yl)-N,N- dimethylpicolinamide | (400 MHz, METHANOL-d₄) δ 8.16-8.09 (m, 2H), 7.96 (dd, J = 2.0, 8.4 Hz, 1H), 7.89-7.82 (m, 1H), 7.75 (d, J = 7.8 Hz, 1H), 7.70 (t, J = 1.0 Hz, 1H), 7.27 (dd, J = 1.6, 10.4 Hz, 1H), 6.37 (s, 1H), 3.13 (d, J = 1.0 Hz, 6H) | Calculated m/z 465.1, found m/z 465.0 |
| 86 | 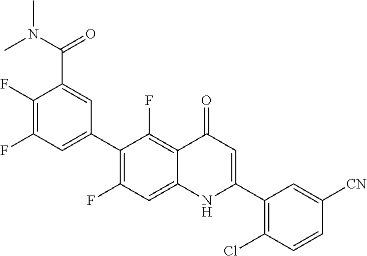 [SMILES: CN(C)C(=O)c1cc(-c2c(F)cc3[nH]c(-c4cc(C#N)ccc4Cl)cc(=O)c3c2F)cc(F)c1F] | 5-(2-(2-chloro- 5-cyanophenyl)- 5,7-difluoro-4- oxo-1,4- dihydroquinolin- 6-yl)-2,3- difluoro-N,N- dimethylbenzamide | (400 MHz, DMSO-d₆) δ 12.42-12.04 (m, 1H), 8.26 (d, J = 1.8 Hz, 1H), 8.10 (dd, J = 2.0, 8.2 Hz, 1H), 7.95 (d, J = 8.4 Hz, 1H), 7.73 (q, J = 7.2 Hz, 1H), 7.43 (t, J = 8.4 Hz, 1H), 7.31 (br d, J = 8.6 Hz, 1H), 6.23-6.10 (m, 1H), 3.10-3.04 (m, 3H), 2.93 (d, J = 6.0 Hz, 3H). | Calculated m/z 500.1, found m/z 500.1 |
| 87 | 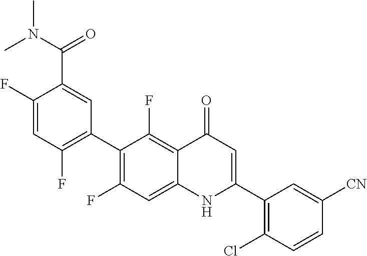 [SMILES: CN(C)C(=O)c1cc(-c2c(F)cc3[nH]c(-c4cc(C#N)ccc4Cl)cc(=O)c3c2F)c(F)cc1F] | 5-(2-(2-chloro- 5-cyanophenyl)- 5,7-difluoro-4- oxo-1,4- dihydroquinolin- 6-yl)-2,4- difluoro-N,N- dimethylbenzamide | (400 MHz, DMSO-d₆) δ 12.35- 12.21 (m, 1H), 8.27 (d, J = 1.6 Hz, 1H), 8.12 (br d, J = 1.8 Hz, 1H), 7.95 (d, J = 8.6 Hz, 1H), 7.71- 7.60 (m, 2H), 7.29 (br d, J = 6.8 Hz, 1H), 6.14 (br s, 1H), 3.03 (s, 3H), 2.91 (s, 3H) | Calculated m/z 500.1, found m/z 500.0 |
| 88 | 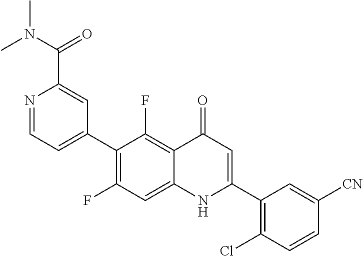 [SMILES: CN(C)C(=O)c1cc(-c2c(F)cc3[nH]c(-c4cc(C#N)ccc4Cl)cc(=O)c3c2F)ccn1] | 4-(2-(2-chloro- 5-cyanophenyl)- 5,7-difluoro-4- oxo-1,4- dihydroquinolin- 6-yl)-N,N- dimethylpicolinamide | (400 MHz, DMSO-d₆) δ = 12.63-11.91 (m, 1H), 8.74 (br d, J = 4.8 Hz, 1H), 8.26 (d, J = 2.0 Hz, 1H), 8.10 (dd, J = 2.0, 8.4 Hz, 1H), 7.49 (d, J = 8.2 Hz, 1H), 7.71 (s, 1H), 7.64 (br d, J = 4.8 Hz, 1H), 7.31 (br d, J = 10.6 Hz, 1H), 6.18 (br s, 1H), 3.06- 3.02 (m, 3H), 3.00 (s, 3H) | Calculated m/z 465.1, found m/z 465.0 |
| 89 | 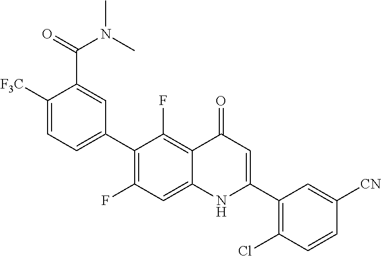 [SMILES: CN(C)C(=O)c1cc(-c2c(F)cc3[nH]c(-c4cc(C#N)ccc4Cl)cc(=O)c3c2F)ccc1C(F)(F)F] | 5-(2-(2-chloro- 5-cyanophenyl)- 5,7-difluoro-4- oxo-1,4- dihydroquinolin- 6-yl)-N,N- dimethyl-2- (trifluoromethyl) benzamide | (400 MHz, METHANOL-d4) δ 8.08 (d, J = 1.9 Hz, 1H), 7.97-7.92 (m, 2H), 7.88-7.78 (m, 2H), 7.63 (s, 1H), 7.28 (dd, J = 1.4, 10.4 Hz, 1H), 6.33 (s, 1H), 3.14 (s, 3H), 2.91 (s, 3H) | Calculated m/z 532.0, found m/z 531.9 |

### Example 90

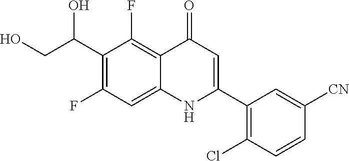 [SMILES: N#Cc1ccc(Cl)c(-c2cc(=O)c3c(F)c(C(O)CO)c(F)cc3[nH]2)c1]

### 4-chloro-3-(6-(1,2-dihydroxyethyl)-5,7-difluoro-4-oxo-1,4-dihdroquinolin-2-yl)benzonitrile

### Step 1, 1-(6-amino-2,4-difluoro-3-vinylphenyl)ethan-1-one

To a mixture of 1-(6-amino-3-bromo-2,4-difluorophenyl)ethan-1-one (500 mg, 2.0 mmol, 1.0 equiv.), potassium trifluoro(vinyl)borate (804 mg, 6.0 mmol, 3.0 equiv.) in dioxane (8 mL) and water (2 mL) was added K₃PO₄ (849 mg, 4.0 mmol, 2.0 equiv.) and Pd(dppf)Cl₂—CH₂Cl₂ (163 mg, 200 mol, 0.1 equiv.). The mixture was degassed with N₂, and then the mixture was stirred at 100° C. for 16 hours under N₂. The reaction mixture was diluted with water (10 mL) and extracted with ethyl acetate (3×10 mL). The combined organic layers were washed with brine (30 mL), dried over Na₂SO₄, filtered, and concentrated under reduced pressure. The residue was purified by silica gel column chromatography (0-40% ethyl acetate in petroleum ether) to afford the title compound (380 mg, 96% yield) as a white solid. LCMS [M+1]=198.1. ¹H NMR (400 MHz, CDCl₃) δ 6.6 (dd, J=18.0, 12.0 Hz, 1H), 6.5 (br s, 2H), 6.2 (dd, J=12.6, 1.6 Hz, 1H), 5.8 (d, J=18.0 Hz, 1H), 5.4 (d, J=12.0 Hz, 1H), 2.6 (d, J=9.0 Hz, 3H).

### Step 2, N-(2-acetyl-3,5-difluoro-4-vinylphenyl)-2-chloro-5-cyanobenzamide

To a solution of 1-(6-amino-2,4-difluoro-3-vinylphenyl)ethan-1-one (380 mg, 1.9 mmol, 1.0 equiv.) in THF (4 mL) was added NaH (77.0 mg, 1.9 mmol, 60% purity, 1.0 equiv.) at 0° C. Then a solution of 2-chloro-5-cyano-benzoyl chloride (424 mg, 2.1 mmol, 1.1 equiv.) in THF (3 mL) was added to the mixture. The mixture was degassed with N₂ and then stirred at 20° C. for 16 hours under N₂. The reaction mixture was quenched by the addition of a saturated aqueous solution of NH₄Cl (10 mL) at 20° C. The mixture was then diluted with water (5 mL) and extracted with ethyl acetate (3×15 mL). The combined organic layers were washed with brine (30 mL), dried over Na₂SO₄, filtered, and concentrated under reduced pressure. The crude product was triturated with acetonitrile (5 mL) at 20° C. to afford the title compound (535 mg, 77% yield) as a white solid. ¹H NMR (400 MHz, DMSO-d₆) δ ppm 11.1 (s, 1H), 8.1 (d, J=1.8 Hz, 1H), 8.0 (dd, J=8.4, 2.0 Hz, 1H), 7.8 (d, J=8.4 Hz, 1H), 7.5 (d, J=11.2 Hz, 1H), 6.7 (dd, J=18.0, 11.8 Hz, 1H), 6.0 (d, J=18.2 Hz, 1H), 5.7 (d, J=11.8 Hz, 1H), 2.6 (d, J=4.0 Hz, 3H).

### Step 3, 4-chloro-3-(5,7-difluoro-4-oxo-6-vinyl-1,4-dihydroquinolin-2-yl)benzonitrile

To a solution of N-(2-acetyl-3,5-difluoro-4-vinylphenyl)-2-chloro-5-cyanobenzamide (100 mg, 277 μmol, 1.0 equiv.) in dioxane (2 mL) was added NaOH (11.1 mg, 277 μmol, 1.0 equiv.). The mixture was stirred at 110° C. for 2 hours. The pH of the reaction mixture was adjusted to 3 with 1 M HCl (1 M). The mixture was diluted with water (10 mL) which precipitated a yellow solid that was isolated by filtration. The filter cake was triturated with water (10 mL). The resulting solid was triturated with acetonitrile (20 mL) at 50° C. for 1 hour to afford the title compound (50 mg, 53% yield) as an off-white solid. LCMS [M+1]=343.0. ¹H NMR (400 MHz, DMSO-d₆) δ 12.0-12.3 (m, 1H), 8.2 (s, 1H), 8.1 (dd, J=8.4, 1.8 Hz, 1H), 7.9 (d, J=8.4 Hz, 1H), 7.1-7.2 (m, 1H), 6.7 (dd, J=17.8, 12.0 Hz, 1H), 6.0 (br d, J=17.8 Hz, 2H), 5.7 (br d, J=12.0 Hz, 1H).

### Step 4, 4-chloro-3-(6-(1,2-dihydroxyethyl)-5,7-difluoro-4-oxo-1,4-dihydroquinolin-2-yl)benzonitrile

To a mixture of K₃[Fe(CN)₆] (86 mg, 263 μmol, 72 uL, 3.0 equiv.), K₂CO₃ (36.3 mg, 262.6 μmol, 3.0 equiv.), DABCO (19.3 μL, 175 μmol, 2.0 equiv.) and K₂OsO₄·2H₂O (32.3 mg, 87.5 μmol, 1.0 equiv.) in t-BuOH (2 mL) and water (2 mL) was added 4-chloro-3-(5,7-difluoro-4-oxo-6-vinyl-1,4-dihydroquinolin-2-yl)benzonitrile (30 mg, 88 μmol, 1.0 equiv.) at 0° C. The mixture was stirred at 20° C. for 3 hours under N₂. The solution was diluted with ethyl acetate (25 mL), quenched with Na₂SO₃ (1 g) and stirred for 10 minutes. The mixture was extracted with ethyl acetate (3×10 mL). The combined organic layers were washed with an aqueous solution of 10% HCl (40 mL), saturated NaHCO₃ (40 mL), dried over Na₂SO₄, filtered, and concentrated under reduced pressure. The residue was purified by preparative HPLC (column: Waters Xbridge BEH C18 100×30 mm×10 um; mobile phase: 1-30% acetonitrile in water (+10 mM NH₄HCO₃)) to afford the title compound (21.0 mg, 63% yield) as a white solid. LCMS [M+1]=377.0. ¹H NMR (400 MHz, DMSO-d₆) δ 8.2 (d, J=2.0 Hz, 1H), 8.1 (dd, J=8.4, 2.0 Hz, 1H), 7.9 (d, J=8.6 Hz, 1H), 7.0-7.1 (m, 1H), 6.0-6.1 (m, 1H), 5.5 (d, J=4.8 Hz, 1H), 4.9-5.0 (m, 1H), 4.9 (t, J=5.8 Hz, 1H), 3.7-3.8 (m, 1H), 3.6 (dt, J=10.8, 6.6 Hz, 1H), 3.3 (s, 1H).

### Example 91

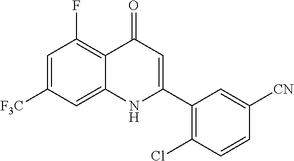 [SMILES: N#Cc1ccc(Cl)c(-c2cc(=O)c3c(F)cc(C(F)(F)F)cc3[nH]2)c1]

### 4-chloro-3-(5-fluoro-4-oxo-7-(trifluoromethyl)-1,4-dihydroquinolin-2-yl)benzonitrile

### Step 1, 4-bromo-3-fluoro-2-iodo-5-(trifluoromethyl)aniline

N-iodosuccinimide (1.1 g, 4.9 mmol, 1.1 equiv.) was added to a solution of 4-bromo-3-fluoro-5-(trifluoromethyl)aniline (1.1 g, 4.4 mmol, 1 equiv.) in acetic acid (10 mL) at 20° C. Then the solution was stirred at 60° C. for 16 hours. The mixture was quenched with a saturated aqueous solution of Na₂SO₃ (20 mL) and extracted with ethyl acetate (3×20 mL). The combined organic extracts were washed with brine (10 mL), dried over Na₂SO₄, filtered and concentrated under reduced pressure. The residue was purified by silica gel column chromatography (80:1 to 20:1 petroleum ether:ethyl acetate)) to afford the title compound (1.06 g, 62% yield) as a yellow solid. ¹H NMR (400 MHz, CHLOROFORM-d) δ 6.89 (s, 1H), 4.66-4.40 (m, 2H).

### Step 2, 1-(6-amino-3-bromo-2-fluoro-4-(trifluoromethyl)phenyl)ethan-1-one

Pd(PPh₃)₄ (301 mg, 260 μmol, 0.1 equiv.) was added to a solution of 4-bromo-3-fluoro-2-iodo-5-(trifluoromethyl)aniline (1 g, 2.6 mmol, 1.0 equiv.) and tributyl(1-ethoxyvinyl)stannane (1.1 g, 3.1 mmol, 1.0 mL, 1.2 equiv.) in toluene (12 mL) at 20° C. The solution was stirred at 120° C. for 16 hours. The solution was quenched with aqueous KF. The mixture was stirred at 20° C. for 1 hour. The mixture was filtered, and the filtrate was extracted with ethyl acetate (3×30 mL). The combined organic layers were washed with water (2×25 mL), brine (15 mL), dried over Na₂SO₄, filtered, and concentrated to give 4-bromo-2-(1-ethoxyvinyl)-3-fluoro-5-(trifluoromethyl)-aniline (900 mg, crude) as a brown oil. Crude 4-bromo-2-(1-ethoxyvinyl)-3-fluoro-5-(trifluoromethyl)aniline (900 mg, 2.1 mmol, 80% purity, 1.0 equiv.) was then treated with HCl (10 mL; 4M in dioxane) and the mixture was stirred at 20° C. for 1 hour. The mixture was concentrated to remove the solvent and the residue was purified by preparative TLC (5:1 petroleum ether:ethyl acetate) to afford the title compound (395 mg, 52% yield) as a yellow solid.

### Step 3, 1-(2-amino-6-fluoro-4-(trifluoromethyl)phenyl)ethan-1-one

1-(6-amino-3-bromo-2-fluoro-4-(trifluoromethyl)phenyl)ethan-1-one (200 mg, 667 μmol, 1 equiv.) was added to a suspension of 10% Pd/C (10 mg, 67 μmol, 0.1 equiv.) in i-PrOH (15 mL) at 20° C. The solution was stirred under an atmosphere of H₂ (50 psi) at 65° C. for 24 hours. The mixture was filtered and the filtrate was concentrated. The residue was purified by preparative TLC (5:1 petroleum ether:ethyl acetate) to afford the title compound (120 mg, 81% yield) as a yellow solid. ¹H NMR (400 MHz, CHLOROFORM-d) δ 6.69 (s, 1H), 6.57 (dd, J=1.2, 11.8 Hz, 1H), 6.52-6.24 (m, 2H), 2.64 (d, J=8.2 Hz, 3H).

### Steps 4 and 5, 4-chloro-3-(5-fluoro-4-oxo-7-(trifluoromethyl)-1,4-dihydroquinolin-2-yl)benzonitrile

Steps 4 and 5 were performed in similar manner to those described for steps 2 and 3 and in the preparation of example 1 using 1-(2-amino-6-fluoro-4-(trifluoromethyl)phenyl)ethan-1-one as the starting material. The title compound was obtained as a white solid. LCMS [M+1]=366.9. ¹H NMR (400 MHz, METHANOL-d₄) δ 8.09 (d, J=1.8 Hz, 1H), 7.94 (br d, J=2.0 Hz, 1H), 7.87-7.83 (m, 1H), 7.71 (br s, 1H), 7.40-7.33 (m, 1H), 6.36 (br s, 1H).

### Example 92

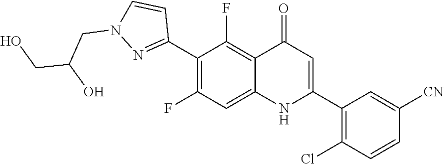 [SMILES: N#Cc1ccc(Cl)c(-c2cc(=O)c3c(F)c(-c4ccn(CC(O)CO)n4)c(F)cc3[nH]2)c1]

### 4-chloro-3-(6-(1-(2,3-dihydroxypropyl)-1H-pyrazol-3-yl)-5,7-difluoro-4-oxo-1,4-dihydroquinolin-2-yl)benzonitrile

The precursor to the title compound, 3-(6-(1-allyl-1H-pyrazol-3-yl)-5,7-difluoro-4-oxo-1,4-dihydroquinolin-2-yl)-4-chlorobenzonitrile, was prepared following Scheme 4.

4-chloro-3-(6-(1-(2,3-dihydroxypropyl)-1H-pyrazol-3-yl)-5,7-difluoro-4-oxo-1,4-dihydroquinolin-2-yl)benzonitrile: To a solution of 3-(6-(1-allyl-1H-pyrazol-3-yl)-5,7-difluoro-4-oxo-1,4-dihydroquinolin-2-yl)-4-chlorobenzonitrile (52.2 mg, 142 μmol, 1.0 equiv.), 1,4-diazabicyclo[2.2.2]octane (31.8 mg, 284 μmol, 31 uL, 2.0 equiv.), K₂CO₃ (59 mg, 426 μmol, 3.0 equiv.), K₃[Fe(CN)₆] (140 mg, 426 μmol, 117 uL, 3.0 equiv.), and K₂OsO₄·2H₂O (51.6 mg, 140 μmol, 1.0 equiv.) in t-BuOH (1.0 mL) and water (1.0 mL) was added 3-[6-(1-allylpyrazol-3-yl)-5,7-difluoro-4-oxo-1H-quinolin-2-yl]-4-chloro-benzonitrile (60 mg, 142 μmol, 1.0 equiv.). The mixture solution was stirred at 20° C. for 2 hours. The mixture was concentrated under reduced pressure. The residue was purified by preparative HPLC (column: Phenomenex Luna 80×30 mm×3 um; mobile phase: 20%-50% acetonitrile in water (+0.04% HCl)) to afford the title compound (11.1 mg, 17% yield) as a yellow solid. LCMS [M+1]=457.0. ¹H NMR (400 MHz, METHANOL-d₄) δ 7.32 (d, J=1.8 Hz, 1H), 7.18 (dd, J=2.0, 8.4 Hz, 1H), 7.07 (d, J=8.4 Hz, 1H), 7.03 (d, J=2.4 Hz, 1H), 6.57 (dd, J=1.4, 10.2 Hz, 1H), 5.90-5.81 (m, 2H), 3.62 (dd, J=4.0, 14.0 Hz, 1H), 3.49-3.39 (m, 1H), 3.29-3.19 (m, 1H), 2.80-2.68 (m, 2H).

### Example 93

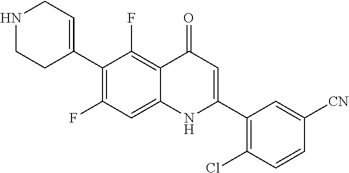 [SMILES: N#Cc1ccc(Cl)c(-c2cc(=O)c3c(F)c(C4=CCNCC4)c(F)cc3[nH]2)c1]

### 4-chloro-3-(5,7-difluoro-4-oxo-6-(1,2,3,6-tetrahydropyridin-4-yl)-14-dihydroquinolin-2-yl)benzonitrile

The precursor to the title compound, tert-butyl 4-(2-(2-chloro-5-cyanophenyl)-5,7-difluoro-4-oxo-1,4-dihydroquinolin-6-yl)-3,6-dihydropyridine-1(2H)-carboxylate, was prepared following Scheme 4 using tert-butyl 4-(4,4,5,5-tetramethyl-1,3,2-dioxaborolan-2-yl)-3,6-dihydropyridine-1(2H)-carboxylate as the starting material.

### 4-chloro-3-(5,7-difluoro-4-oxo-6-(1,2,3,6-tetrahydropyridin-4-yl)-14-dihydroquinolin-2-yl)benzonitrile

To a mixture of tert-butyl 4-(2-(2-chloro-5-cyanophenyl)-5,7-difluoro-4-oxo-1,4-dihydroquinolin-6-yl)-3,6-dihydropyridine-1(2H)-carboxylate (140 mg, 281 μmol, 1.0 equiv.) in DCM (4 mL) was added TFA (0.4 mL) at 20° C. under N₂. The mixture was stirred at 20° C. for 1 hour. The reaction mixture was concentrated in vacuum to give a residue. The pH 7-8 of the residue was adjusted to with saturated NaHCO₃ solution and concentrated in vacuum. The residue was purified by preparative HPLC (column: Phenomenex Luna C18 150×30 mm×5 u m; mobile phase: 5-45% acetonitrile in water (0.2% formic acid)) to afford the title compound (22.3 mg, 19% yield) as white solid. LCMS [M+2]=399.0. ¹H NMR (400 MHz, METHANOL-d₄) 6=8.48 (d, J=2.6 Hz, 1H), 8.05 (s, 1H), 7.98-7.90 (m, 1H), 7.83 (d, J=8.4 Hz, 1H), 7.17 (d, J=10.4 Hz, 1H), 6.29 (s, 1H), 6.02 (br s, 1H), 3.90 (br d, J=1.0 Hz, 2H), 3.49 (t, J=5.8 Hz, 2H), 2.78-2.68 (m, 2H).

### Example 94

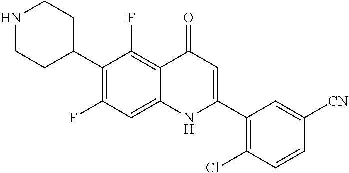 [SMILES: N#Cc1ccc(Cl)c(-c2cc(=O)c3c(F)c(C4CCNCC4)c(F)cc3[nH]2)c1]

### 4-chloro-3-(5,7-difluoro-4-oxo-6-(piperidin-4-yl)-1,4-dihydroquinolin-2-yl)benzonitrile

The precursor to the title compound, tert-butyl 4-(2-(2-chloro-5-cyanophenyl)-5,7-difluoro-4-oxo-1,4-dihydroquinolin-6-yl)piperidine-1-carboxylate, was prepared following Scheme 4 using tert-butyl 4-(4,4,5,5-tetramethyl-1,3,2-dioxaborolan-2-yl)-3,6-dihydropyridine-1(2H)-carboxylate as the starting material.

To a solution of tert-butyl 4-(2-(2-chloro-5-cyanophenyl)-5,7-difluoro-4-oxo-1,4-dihydroquinolin-6-yl)piperidine-1-carboxylate (170 mg, 340 μmol, 1 equiv.) in DCM (3 mL) was added TFA (0.3 mL, 4.1 mmol, 11.9 equiv.) at 20° C. The mixture was stirred at 20° C. for 1 hour. The reaction mixture was concentrated in vacuum to give a residue. The pH of the residue was adjusted to 7-8 with NaHCO₃ (aq.), then the mixture was concentrated in vacuum. The residue was purified by preparative HPLC (column: Phenomenex Luna C18 150×30 mm×5 um; mobile phase: 5-40% acetonitrile in water (0.2% formic acid)) to afford the title compound (63 mg, 45% yield) as a white solid. LCMS [M+1]=400.0. ¹H NMR (400 MHz, METHANOL-d₄) δ 8.04 (d, J=2.0 Hz, 1H), 7.93 (dd, J=2.1, 8.4 Hz, 1H), 7.83 (d, J=8.6 Hz, 1H), 7.15 (dd, J=1.5, 11.4 Hz, 1H), 6.28 (s, 1H), 3.57-3.45 (m, 3H), 3.24-3.12 (m, 2H), 2.45-2.30 (m, 2H), 2.05 (br d, J=14.3 Hz, 2H).

### Example 95

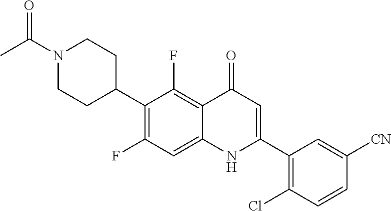 [SMILES: CC(=O)N1CCC(c2c(F)cc3[nH]c(-c4cc(C#N)ccc4Cl)cc(=O)c3c2F)CC1]

### 3-(6-(1-acetylpiperidin-4-yl)-5,7-difluoro-4-oxo-1,4-dihydroquinolin-2-yl)-4-chlorobenzonitrile

To a solution of 4-chloro-3-(5,7-difluoro-4-oxo-6-(piperidin-4-yl)-1,4-dihydroquinolin-2-yl)benzonitrile (40 mg, 100 μmol, 1 equiv.) in DCM (2 mL) was added triethylamine (42 uL, 300 μmol, 3 equiv.) and acetic anhydride (8.4 uL, 90.0 μmol, 0.9 equiv.) at 20° C. The mixture was stirred at 20° C. for 16 hours under N₂. The reaction mixture was concentrated under reduced pressure and the resulting residue was purified by preparative HPLC (column: Phenomenex Luna C18 75×30 mm×3 um; mobile phase: 10-50% acetonitrile in water (0.2% formic acid)) to afford the title compound (17.8 mg, 39% yield) as a white solid. LCMS [M+1]=442.1. ¹H NMR (400 MHz, METHANOL-d₄) δ 8.05 (d, J=1.7 Hz, 1H), 7.93 (dd, J=1.7, 8.4 Hz, 1H), 7.83 (d, J=8.3 Hz, 1H), 7.11 (br d, J=11.4 Hz, 1H), 6.25 (s, 1H), 4.71 (br d, J=13.1 Hz, 1H), 4.08 (br d, J=13.6 Hz, 1H), 3.52-3.38 (m, 1H), 3.29-3.21 (m, 1H), 2.74 (br t, J=12.0 Hz, 1H), 2.16 (s, 3H), 2.14-1.95 (m, 2H), 1.92-1.77 (m, 2H).

### Example 96

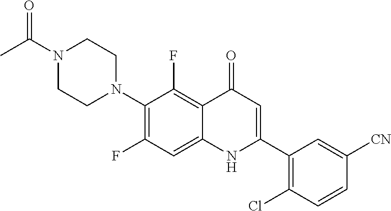 [SMILES: CC(=O)N1CCN(c2c(F)cc3[nH]c(-c4cc(C#N)ccc4Cl)cc(=O)c3c2F)CC1]

### 3-(6-(4-acetylpiperazin-1-yl)-5,7-difluoro-4-oxo-1,4-dihydroquinolin-2-yl)-4-chlorobenzonitrile

The precursor to the title compound, tert-butyl 4-(2-(2-chloro-5-cyanophenyl)-5,7-difluoro-4-oxo-1,4-dihydroquinolin-6-yl)piperazine-1-carboxylate, was prepared following Scheme 1 using tert-butyl 4-(4-amino-2,6-difluorophenyl)piperazine-1-carboxylate as the starting material.

### Step 1, 4-chloro-3-(5,7-difluoro-4-oxo-6-(piperazin-1-yl)-1,4-dihydroquinolin-2-yl)benzonitrile

To a solution of tert-butyl 4-(2-(2-chloro-5-cyanophenyl)-5,7-difluoro-4-oxo-1,4-dihydroquinolin-6-yl)piperazine-1-carboxylate (83 mg, 166 μmol, 1.0 equiv.) in HCl/dioxane (2.5 mL). The mixture was stirred at 20° C. for 1 hour. The mixture was extracted with ethyl acetate (3×10 mL). The combined organic layers were washed with brine (20 mL), dried over Na₂SO₄, filtered, and concentrated. The residue was purified by preparative HPLC (column: Phenomenex Luna 80×30 mm×3 um; mobile phase: 5-40% acetonitrile in water (0.04% HCl)) to afford the title compound (46.6 mg, 66% yield) as a yellow solid. LCMS [M+1]=401.1. ¹H NMR (400 MHz, METHANOL-d₄) δ 8.12 (d, J=2.0 Hz, 1H), 8.00 (dd, J=2.0, 8.4 Hz, 1H), 7.89 (d, J=8.6 Hz, 1H), 7.37 (dd, J=1.8, 11.2 Hz, 1H), 6.68 (s, 1H), 3.59-3.53 (m, 4H), 3.44-3.37 (m, 4H).

### Step 2, 3-(6-(4-acetylpiperazin-1-yl)-5,7-difluoro-4-oxo-1,4-dihydroquinolin-2-yl)-4-chlorobenzonitrile

To a solution of 4-chloro-3-(5,7-difluoro-4-oxo-6-(piperazin-1-yl)-1,4-dihydroquinolin-2-yl)benzonitrile (30 mg, 74.8 μmol, 1.0 equiv.) in DCM (1.5 mL) was added triethylamine (31 uL, 225 μmol, 3.0 equiv.) and acetic anhydride (6.3 uL, 67 μmol, 0.9 equiv.). The mixture was stirred at 20° C. for 2 hours. The reaction solution was concentrated under reduced pressure and the resulting residue was purified by preparative HPLC (column: Phenomenex Luna C18 75×30 mm×3 um; mobile phase: 10-50% acetonitrile in water (+0.2% formic acid)) to afford the title compound (17.2 mg, 50% yield) as a yellow solid. LCMS [M+1]=443.1.

¹H NMR (400 MHz, METHANOL-d₄) δ 8.05 (d, J=2.0 Hz, 1H), 7.95-7.91 (m, 1H), 7.85-7.81 (m, 1H), 7.14 (dd, J=1.8, 11.6 Hz, 1H), 6.27 (s, 1H), 3.72 (td, J=5.2, 19.0 Hz, 4H), 3.29-3.18 (m, 4H), 2.16 (s, 3H).

### Example 97

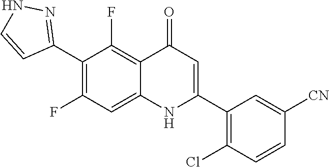 [SMILES: N#Cc1ccc(Cl)c(-c2cc(=O)c3c(F)c(-c4cc[nH]n4)c(F)cc3[nH]2)c1]

### 4-chloro-3-(5,7-difluoro-4-oxo-6-(1H-pyrazol-3-yl)-1,4-dihydroquinolin-2-yl)benzonitrile

### Step 1, 1-(6-amino-2,4-difluoro-3-(1-(tetrahydro-2H-pyran-2-yl)-1H-pyrazol-3-yl)phenyl)ethan-1-one

To a solution of 1-(6-amino-2,4-difluoro-3-iodophenyl)ethan-1-one (350 mg, 1.2 mmol, 1 equiv.) and 1-(tetrahydro-2H-pyran-2-yl)-3-(4,4,5,5-tetramethyl-1,3,2-dioxaborolan-2-yl)-1H-pyrazole (492 mg, 1.8 mmol, 1.5 equiv.) in DME (7 mL)/EtOH (7 mL)/H₂O (1.4 mL) was added Na₂CO₃ (375 mg, 3.5 mmol, 3 equiv.) and Pd(PPh₃)₂Cl₂ (82.7 mg, 118 μmol, 0.1 equiv.). The mixture was stirred at 80° C. for 1 hour with microwave irradiation under N₂. The reaction was diluted with water (20 mL) and the aqueous phase was extracted with ethyl acetate (2×35 mL). The combined organic layers were washed with brine (5 mL), dried with anhydrous Na₂SO₄, filtered, and concentrated in vacuum. The residue was purified by preparative HPLC (column: Phenomenex Gemini-NX 80×40 mm×3 um; mobile phase: 20-50% acetonitrile in water (+10 mM NH₄HCO₃)) to afford the title compound (200 mg, 53% yield) as a white solid. ¹H NMR (400 MHz, DMSO-d₆) δ 7.79 (br s, 2H), 7.63 (d, J=1.8 Hz, 1H), 6.62-6.48 (m, 1H), 6.39 (d, J=1.8 Hz, 1H), 5.11 (br d, J=8.8 Hz, 1H), 3.90-3.72 (m, 1H), 3.55-3.38 (m, 1H), 2.53 (br s, 3H), 2.30-2.19 (m, 1H), 2.01-1.90 (m, 1H), 1.84-1.73 (m, 1H), 1.70-1.55 (m, 1H), 1.54-1.41 (m, 2H).

### Step 2, N-(2-acetyl-4-(1-(2-chloro-5-cyanobenzoyl)-1H-pyrazol-3-yl)-3,5-difluorophenyl)-2-chloro-5-cyanobenzamide

To a solution of 1-(6-amino-2,4-difluoro-3-(1-(tetrahydro-2H-pyran-2-yl)-1H-pyrazol-3-yl)phenyl)ethan-1-one (180 mg, 560 μmol, 1.0 equiv.) in THF (5 mL) was added NaH (67.2 mg, 1.7 mmol, 3.0 equiv.; 60% dispersion in oil) at 0° C. Then 2-chloro-5-cyanobenzoyl chloride (336 mg, 1.7 mmol, 3.0 equiv.) was added to the reaction and the mixture was stirred at 20° C. for 16 hours under N₂. The residue was poured into water (10 mL) slowly. The aqueous solution was extracted with ethyl acetate. The organic layer was dried over Na₂SO₄, filtered, and concentrated under reduced pressure to afford the title compound (160 mg, 51% yield) as a white solid. ¹H NMR (400 MHz, DMSO-d₆) δ 11.21 (s, 1H), 8.80 (d, J=2.8 Hz, 1H), 8.39 (d, J=2.0 Hz, 1H), 8.13-8.08 (m, 1H), 8.04 (dd, J=2.0, 8.4 Hz, 1H), 7.90 (d, J=8.6 Hz, 1H), 7.86-7.80 (m, 1H), 7.54 (br d, J=10.6 Hz, 1H), 7.08 (br s, 1H), 2.54 (d, J=3.8 Hz, 3H).

### Step 3, N-(2-acetyl-3,5-difluoro-4-(1H-pyrazol-3-yl)phenyl)-2-chloro-5-cyanobenzamide

To a solution of N-[2-acetyl-4-[1-(2-chloro-5-cyano-benzoyl)pyrazol-3-yl]-3,5-difluoro-phenyl]-2-chloro-5-cyano-benzamide (160 mg, 284 μmol, 1.0 equiv.) in MeOH (3 mL) was added K₂CO₃ (79 mg, 567 μmol, 2.0 equiv.). The mixture was stirred at 20° C. for 1 hour. The residue was poured into water (10 mL). The aqueous phase was extracted with ethyl acetate (2×25 mL). The combined organic layers were washed with brine (10 mL), dried with Na₂SO₄, filtered, and concentrated under reduced pressure. The residue was purified by silica gel chromatography (33% to 100% ethyl acetate in petroleum ether) to afford the title compound (80 mg, 70% yield) as a white solid. ¹H NMR (400 MHz, DMSO-d₆) δ 13.27 (br s, 1H), 11.21-11.04 (m, 1H), 8.14 (d, J=1.8 Hz, 1H), 8.09-8.04 (m, 1H), 7.92 (s, 1H), 7.89-7.84 (m, 1H), 7.54 (br d, J=11.4 Hz, 1H), 6.59 (br s, 1H), 2.59 (br d, J=3.8 Hz, 3H).

### Step 4, 4-chloro-3-(5,7-difluoro-4-oxo-6-(1H-pyrazol-3-yl)-1,4-dihydroquinolin-2-yl)benzonitrile

To a solution of N-(2-acetyl-3,5-difluoro-4-(1H-pyrazol-3-yl)phenyl)-2-chloro-5-cyanobenzamide (50 mg, 125 μmol, 1.0 equiv.) in dioxane (1.5 mL) was added NaOH (49.9 mg, 1.3 mmol, 10 equiv.). The mixture was stirred at 110° C. for 1 hour. The pH of the reaction mixture was adjusted to 6-7 with 1M aqueous HCl. The mixture was diluted with water (5 mL) and the aqueous phase was extracted with ethyl acetate (2×20 mL). The combined organic layers were washed with brine (3 mL), dried with Na₂SO₄, filtered, and concentrated under reduced pressure. The residue was purified by preparative HPLC (column: Phenomenex Gemini-NX C18 75×30 mm×3 um; mobile phase: 10-40% acetonitrile in water (+10 mM NH₄HCO₃)). Then the product was further purified by preparative TLC (10:1 dichloromethane:methanol). The isolated product was then triturated with MTBE (3 mL) to afford the title compound (2.8 mg, 5.9% yield) as an off-white solid. LCMS [M+1]=383.1. ¹H NMR (400 MHz, DMSO-d₆) δ 13.35-13.14 (m, 1H), 12.27-12.16 (m, 1H), 8.25 (br s, 1H), 8.08 (br dd, J=1.6, 8.4 Hz, 1H), 7.93 (br d, J=8.6 Hz, 1H), 7.89-7.77 (m, 1H), 7.24 (br d, J=10.4 Hz, 1H), 6.58 (br s, 1H), 6.09 (br s, 1H).

### Example 98

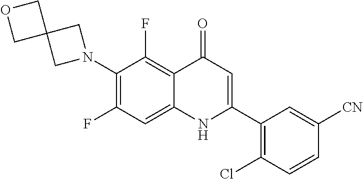 [SMILES: N#Cc1ccc(Cl)c(-c2cc(=O)c3c(F)c(N4CC5(COC5)C4)c(F)cc3[nH]2)c1]

### 4-chloro-3-(5,7-difluoro-4-oxo-6-(2-oxa-6-azaspiro[3.3]heptan-6-yl)-14-dihydroquinolin-2-yl)benzonitrile

To a solution of 4-chloro-3-(5,7-difluoro-6-iodo-4-oxo-1,4-dihydroquinolin-2-yl)benzonitrile (200 mg, 452 μmol, 1.0 equiv.) and 2-oxa-6-azaspiro[3.3]heptane oxalic acid salt (86 mg, 452 μmol, 1.0 equiv.) in DMF (4 mL) was added Cs₂CO₃ (736 mg, 2.3 mmol, 5 equiv.), (5-diphenylphosphanyl-9,9-dimethyl-xanthen-4-yl)-diphenyl-phosphane (52 mg, 90 mol, 0.2 equiv.) and Pd₂dba₃ (41.4 mg, 45 μmol, 0.1 equiv.). The mixture was stirred at 120° C. for 1 hour under N₂. The residue was poured into water (20 mL) and the aqueous phase was extracted with ethyl acetate (20 mL). The organic layer was dried over Na₂SO₄, filtered, and concentrated. The mixture was purified by preparative HPLC (column: Phenomenex Luna C18 100×30 mm×5 um; mobile phase: 10-50% acetonitrile in water (+0.2% formic acid)) to afford the title compound (31.2 mg, 16% yield) as a yellow solid. LCMS [M+1]=414.0.

¹H NMR (400 MHz, DMSO-d₆) δ=12.00-11.75 (m, 1H), 8.21 (s, 1H), 8.07 (br d, J=7.6 Hz, 1H), 7.91 (br d, J=8.4 Hz, 1H), 7.06 (br d, J=12.8 Hz, 1H), 5.93 (s, 1H), 4.73 (s, 4H), 4.33 (br s, 4H).

### Example 99

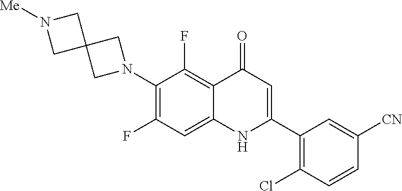 [SMILES: CN1CC2(C1)CN(c1c(F)cc3[nH]c(-c4cc(C#N)ccc4Cl)cc(=O)c3c1F)C2]

### 4-chloro-3-(5,7-difluoro-6-(6-methyl-2,6-diazaspiro[3.3]heptan-2-yl)-4-oxo-1,4-dihydroquinolin-2-yl)benzonitrile

### Step 1, tert-butyl 6-(2-(2-chloro-5-cyanophenyl)-5,7-difluoro-4-oxo-1,4-dihydroquinolin-6-yl)-2,6-diazaspiro[3.3]heptane-2-carboxylate

This compound was prepared in a similar manner to Example 98 using N,N-dimethylazetidin-3-amine-dihydrochloride as a starting material. ¹H NMR (400 MHz, METHANOL-d₄) δ 8.01 (d, J=1.8 Hz, 1H), 7.91 (dd, J=1.8, 8.4 Hz, 1H), 7.83-7.76 (m, 1H), 7.42-7.33 (m, 1H), 7.07 (br d, J=13.0 Hz, 1H), 6.18 (br s, 1H), 4.37 (br s, 4H), 4.11 (s, 4H), 1.45 (s, 9H).

### Step 2, 4-chloro-3-(5,7-difluoro-4-oxo-6-(2,6-diazaspiro[3.3]heptan-2-yl)-14-dihydroquinolin-2-yl)benzonitrile

A solution of tert-butyl 6-(2-(2-chloro-5-cyanophenyl)-5,7-difluoro-4-oxo-1,4-dihydroquinolin-6-yl)-2,6-diazaspiro[3.3]heptane-2-carboxylate (100 mg, 195 μmol, 1.0 equiv.) in TFA (0.6 mL) and DCM (2 mL) was stirred at 20° C. for 1 hour under N₂. The reaction mixture was concentrated under reduced pressure to give a residue. The residue was purified by preparative HPLC (column: Phenomenex Luna C18 75×30 mm×3 um; mobile phase: 1-40% acetonitrile in water (+0.2% formic acid)) to afford the title compound (27 mg, 34% yield) as yellow solid. ¹H NMR (400 MHz, METHANOL-d₄) δ 8.54 (s, 1H), 8.00 (d, J=2.0 Hz, 1H), 7.94-7.86 (m, 1H), 7.84-7.77 (m, 1H), 7.10 (dd, J=1.8, 13.2 Hz, 1H), 6.23 (s, 1H), 4.45 (t, J=2.4 Hz, 4H), 4.27 (s, 4H).

### Step 3, 4-chloro-3-(5,7-difluoro-6-(6-methyl-2,6-diazaspiro[3.3]heptan-2-yl)-4-oxo-1,4-dihydroquinolin-2-yl)benzonitrile

To a solution of 4-chloro-3-(5,7-difluoro-4-oxo-6-(2,6-diazaspiro[3.3]heptan-2-yl)-1,4-dihydroquinolin-2-yl)benzonitrile (19 mg, 46 μmol, 1.0 equiv.) in MeOH (0.5 mL) was added NaBH(OAc)₃ (29.2 mg, 138 μmol, 3.0 equiv.), acetic acid (7.9 uL, 138 μmol, 3 equiv.), and formaldehyde (10.3 uL, 138 μmol, 3.0 equiv.; 37% in water) at 20° C. under N₂. The mixture was stirred at 20° C. for 4 hours. The solution was directly purified by preparative HPLC (column: Phenomenex Luna C18 75×30 mm×3 um; mobile phase: 1-40% acetonitrile in water (+0.2% formic acid)) to afford the title compound (4.1 mg, 20% yield) as yellow solid. LCMS [M+1]=427.0. ¹H NMR (400 MHz, METHANOL-d₄) δ 8.53 (br s, 1H), 8.00 (d, J=1.8 Hz, 1H), 7.90 (dd, J=1.8, 8.4 Hz, 1H), 7.86-7.74 (m, 1H), 7.09 (br d, J=12.8 Hz, 1H), 6.21 (s, 1H), 4.43 (br s, 4H), 4.19 (s, 4H), 2.80 (s, 3H).

The compounds in Table 8 were prepared using similar procedure described for Example 98.

TABLE 8

| | | IUPAC | | LCMS |
|---|---|---|---|---|
| Example number | Structure | Name | ¹H NMR | Exact Mass |
| 100 | 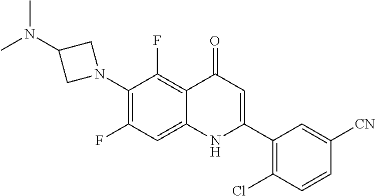 [SMILES: CN(C)C1CN(c2c(F)cc3[nH]c(-c4cc(C#N)ccc4Cl)cc(=O)c3c2F)C1] | 4-chloro-3-(6-(3- (dimethylamino) azetidin-1-yl)- 5,7-difluoro-4-oxo-1,4- dihydroquinolin- 2-yl) benzonitrile | (400 MHz, DMSO-d₆) δ 12.07- 11.68 (br s, 1H), 8.24-8.13 (m, 1H), 8.05 (br d, J = 8.0 Hz, 1H), 7.89 (br d, J = 8.2 Hz, 1H), 7.08 (br s, 1H), 6.10-5.73 (m, 1H), 4.24 (br s, 2H), 3.93 (br s, 2H), 3.18-3.09 (m, 1H), 2.11 (s, 6H). | Calculated m/z 415.1, found m/z 415.0 |
| 101 | 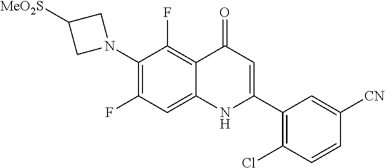 [SMILES: CS(=O)(=O)C1CN(c2c(F)cc3[nH]c(-c4cc(C#N)ccc4Cl)cc(=O)c3c2F)C1] | 4-chloro-3-(5,7- difluoro-6-(3- (methylsulfonyl) azetidin-1-yl)- 4-oxo-1,4- dihydroquinolin- 2-yl)benzonitrile | (400 MHz, DMSO-d₆) δ 11.90 (s, 1H), 8.21 (d, J = 1.9 Hz, 1H), 8.07 (dd, J = 1.9, 8.3 Hz, 1H), 7.91 (d, J = 8.4 Hz, 1H), 7.10 (br d, J = 13.1 Hz, 1H), 5.94 (s, 1H), 4.65- 4.33 (m, 5H), 3.10- 3.05 (m, 3H) | Calculated m/z 450.0, found m/z 449.9 |
| 102 | 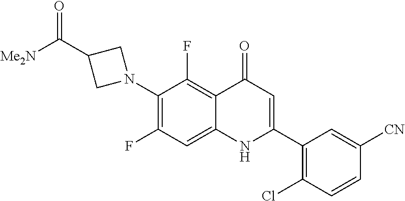 [SMILES: CN(C)C(=O)C1CN(c2c(F)cc3[nH]c(-c4cc(C#N)ccc4Cl)cc(=O)c3c2F)C1] | 1-(2-(2-chloro- 5- cyanophenyl)- 5,7-difluoro-4- oxo-1,4- dihydroquinolin- 6-yl)-N,N- dimethylazetidine- 3-carboxamide | (400 MHz, DMSO-d₆) δ 12.02- 11.70 (m, 1H), 8.28- 8.13 (m, 1H), 8.10- 7.98 (m, 1H), 7.89 (br d, J = 8.4 Hz, 1H), 7.27-6.96 (m, 1H), 6.11-5.78 (m, 1H), 4.48-4.16 (m, 4H), 3.90-3.75 (m, 1H), 2.87 (d, J = 17.8 Hz, 6H) | Calculated m/z 443.1, found m/z 443.1 |
| 103 | 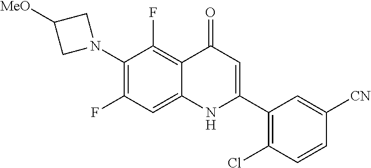 [SMILES: COC1CN(c2c(F)cc3[nH]c(-c4cc(C#N)ccc4Cl)cc(=O)c3c2F)C1] | 4-chloro-3-(5,7- difluoro-6-(3- methoxyazetidin- 1-yl)-4-oxo- 1,4- dihydroquinolin- 2-yl)benzonitrile | (400 MHz, DMSO-d₆) δ 12.01- 11.76 (m, 1H), 8.25- 8.10 (m, 1H), 8.03 (br d, J = 6.4 Hz, 1H), 7.88 (br d, J = 8.2 Hz, 1H), 7.08 (br d, J = 4.6 Hz, 1H), 5.92 (br s, 1H), 4.37 (br s, 2H), 4.25 (quin, J = 5.2 Hz, 1H), 3.96 (br s, 2H), 3.24 (s, 3H) | Calculated m/z 402.1, found m/z 402.1 |

### Example 104

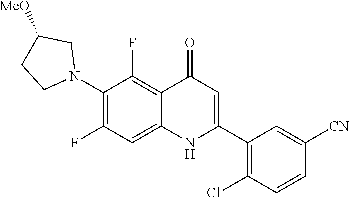 [SMILES: CO[C@H]1CCN(c2c(F)cc3[nH]c(-c4cc(C#N)ccc4Cl)cc(=O)c3c2F)C1]

### (S)-4-chloro-3-(5,7-difluoro-6-(3-methoxypyrrolidin-1-yl)-4-oxo-1,4-dihydroquinolin-2-yl)benzonitrile

### Scheme 6, step 1, (S)-1-(3-bromo-2,6-difluoro-4-nitrophenyl)-3-methoxypyrrolidine

To a solution of 2-bromo-3,4,5-trifluoro-1-nitrobenzene (2 g, 7.81 mmol, 1.0 equiv.) and (S)-3-methoxypyrrolidine hydrochloride (1.18 g, 8.59 mmol, 1.1 equiv.) in DMF (12 mL) was added diisopropylethylamine (5.44 mL, 31.3 mmol, 4 equiv.). The mixture was stirred at 55° C. for 5 hours. The residue was poured into water (30 mL). The aqueous phase was extracted with MTBE (2×100 mL). The combined organic layers were washed with brine (3×100 mL), dried over Na₂SO₄, filtered, and concentrated under reduced pressure to afford the title compound (2.6 g, 98% yield) as a yellow solid. ¹H NMR (400 MHz, CHLOROFORM-d) δ 7.73 (dd, J=1.8, 14.2 Hz, 1H), 4.04 (tt, J=2.0, 4.2 Hz, 1H), 4.01-3.88 (m, 2H), 3.76-3.66 (m, 2H), 3.37 (s, 3H), 2.16 (ddt, J=2.0, 4.4, 8.6 Hz, 1H), 2.00-1.89 (m, 1H).

### Scheme 6, step 2, (S)-2-bromo-3,5-difluoro-4-(3-methoxypyrrolidin-1-yl)aniline

To a solution of (S)-1-(3-bromo-2,6-difluoro-4-nitrophenyl)-3-methoxypyrrolidine (2.5 g, 7.42 mmol, 1 equiv.) in EtOH (16 mL) and water (4 mL) was added iron(0) (2.07 g, 37.1 mmol, 5.0 equiv.) and NH₄Cl (1.98 g, 37.1 mmol, 5.0 equiv.). The mixture was stirred at 80° C. for 20 minutes. The suspension was filtered through a pad of celite and the filter cake was washed with ethyl acetate (2×50 mL). The filtrate was concentrated under reduced pressure and the residue was purified by silica gel chromatography (15:1 to 5:1 petroleum ether:ethyl acetate) to afford the title compound (1.8 g, 79% yield) as a brown solid. ¹H NMR (400 MHz, CHLOROFORM-d) δ 6.32 (dd, J=2.0, 13.0 Hz, 1H), 4.17-3.89 (m, 3H), 3.53-3.41 (m, 2H), 3.35 (s, 3H), 3.31-3.20 (m, 2H), 2.18-2.05 (m, 1H), 1.99 (ddd, J=3.8, 8.2, 12.4 Hz, 1H).

### Scheme 6, step 3, (S)-1-(6-amino-2,4-difluoro-3-(3-methoxypyrrolidin-1-yl)phenyl)ethan-1-one

To a solution of (S)-2-bromo-3,5-difluoro-4-(3-methoxypyrrolidin-1-yl)aniline (1.8 g, 5.86 mmol, 1.0 equiv.) and tributyl(1-ethoxyvinyl)stannane (2.97 mL, 8.79 mmol, 1.5 equiv.) in toluene (27 mL) was added Pd(PPh₃)₄ (677 mg, 586 μmol, 0.1 equiv.). The mixture was stirred at 120° C. for 16 hours. The mixture was added into an aqueous solution of KF (30 mL) and the mixture was stirred for 1 hour. The mixture was then diluted with water (50 mL) and extracted with ethyl acetate (3×50 mL). The combined organic layers were washed with brine (50 mL), dried over sodium sulfate, and concentrated under reduced pressure to afford (S)-2-(1-ethoxyvinyl)-3,5-difluoro-4-(3-methoxypyrrolidin-1-yl)aniline (1.5 g, crude) as a brown solid.

(S)-2-(1-ethoxyvinyl)-3,5-difluoro-4-(3-methoxypyrrolidin-1-yl)aniline (1.2 g, 4.02 mmol, 1 equiv.) was treated with a HCl (10 mL; 4M in dioxane) was stirred at 20° C. for 1 hour. The mixture was concentrated in vacuum. The residue was purified by silica gel chromatography (20:1 to 5:1 petroleum ether:ethyl acetate) to afford the title compound (400 mg, 37% yield) as a white solid. ¹H NMR (400 MHz, CHLOROFORM-d) δ 6.30-6.00 (m, 3H), 4.06 (td, J=3.0, 6.2 Hz, 1H), 3.49-3.39 (m, 2H), 3.39-3.34 (m, 3H), 3.30-3.16 (m, 2H), 2.58 (d, J=8.8 Hz, 3H), 2.14 (td, J=6.8, 13.6 Hz, 1H), 2.00 (ddd, J=3.8, 8.2, 12.4 Hz, 1H).

### Scheme 6, step 3, (S)—N-(2-acetyl-3,5-difluoro-4-(3-methoxypyrrolidin-1-yl)phenyl)-2-chloro-5-cyanobenzamide

To a solution of (S)-1-(6-amino-2,4-difluoro-3-(3-methoxypyrrolidin-1-yl)phenyl)ethan-1-one (120 mg, 444 μmol, 1.0 equiv.) in isopropyl acetate (1.8 mL) was added 2-chloro-5-cyano-benzoyl chloride (98 mg, 488 μmol, 1.1 equiv.). The mixture was stirred at 80° C. for 2 hours under N₂. The mixture was cooled to 20° C. The residue was poured into water (20 mL) and the aqueous phase was extracted with ethyl acetate (2×30 mL). The combined organic layers were washed with brine (10 mL), dried with Na₂SO₄, filtered, and concentrated in vacuum. The crude product was triturated with MTBE (3 mL) to afford the title compound (80 mg, 42% yield) as a yellow solid. ¹H NMR (400 MHz, CHLOROFORM-d) δ=11.62 (s, 1H), 8.45-8.26 (m, 1H), 7.91 (d, J=2.0 Hz, 1H), 7.73-7.67 (m, 1H), 7.65-7.57 (m, 1H), 4.13-4.01 (m, 1H), 3.78-3.67 (m, 2H), 3.45 (br d, J=9.2 Hz, 2H), 3.39 (s, 3H), 2.65 (d, J=8.8 Hz, 3H), 2.12-2.02 (m, 2H).

### Scheme 6, step 4, (S)-4-chloro-3-(5,7-difluoro-6-(3-methoxypyrrolidin-1-yl)-4-oxo-1,4-dihydroquinolin-2-yl)benzonitrile

To a solution of (S)—N-(2-acetyl-3,5-difluoro-4-(3-methoxypyrrolidin-1-yl)phenyl)-2-chloro-5-cyanobenzamide (80 mg, 184 μmol, 1.0 equiv.) in dioxane (1.2 mL) was added LiOH (6.6 mg, 277 μmol, 1.5 equiv.). The mixture was stirred at 110° C. for 16 hours under N₂. The pH of the mixture was adjusted to 4-5 with an aqueous solution of HCl (1 M). The mixture was diluted with water (5 mL) and the aqueous phase was extracted with ethyl acetate (2×30 mL). The combined organic layers were washed with brine (10 mL), dried with Na₂SO₄, filtered, and concentrated under reduced pressure. The crude product was triturated with acetonitrile (1 mL) to afford the title compound (36.6 mg, 47% yield) as a white solid. LCMS [M+1]=416.0. H NMR (400 MHz, METHANOL-d₄) δ 8.03 (d, J 2.0 Hz, 1H), 7.95-7.88 (m, 1H), 7.85-7.77 (m, 1H), 7.09 (br d, J=12.2 Hz, 1H), 6.21 (br s, 1H), 4.10 (td, J=2.4, 5.2 Hz, 1H), 3.84-3.68 (m, 2H), 3.53-3.43 (m, 2H), 3.40-3.35 (m, 3H), 2.15-2.02 (in, 2H).

The compounds in Table 9 were prepared following Scheme 6 using similar procedures as those described for Example 104.

TABLE 9

| | | IUPAC | | LCMS |
|---|---|---|---|---|
| Example number | Structure | Name | ¹H NMR | Exact Mass |
| 105 | 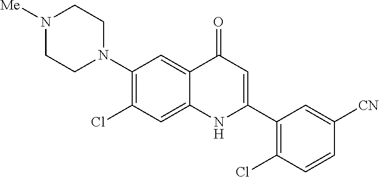 [SMILES: CN1CCN(c2cc3c(=O)cc(-c4cc(C#N)ccc4Cl)[nH]c3cc2Cl)CC1] | 4-chloro-3-(7- chloro-6-(4- methylpiperazin- 1-yl)-4-oxo- 1,4- dihydroquinolin- 2-yl)benzonitrile | (400 MHz, DMSO-d₆) δ 8.22 (d, J = 2.0 Hz, 1H), 8.15 (s, 1H), 8.07 (dd, J = 2.0, 8.4 Hz, 1H), 7.92 (d, J = 8.4 Hz, 1H), 7.74 (s, 1H), 7.67 (s, 1H), 6.17 (br s, 1H), 3.03 (br s, 4H), 2.55 (br d, J = 6.0 Hz, 4H), 2.28 (s, 3H) | Calculated m/z 413.1, found m/z 413.0 |
| 106 | 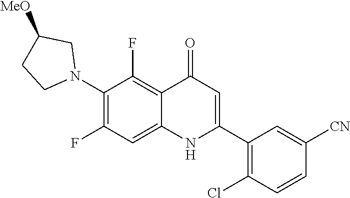 [SMILES: CO[C@@H]1CCN(c2c(F)cc3[nH]c(-c4cc(C#N)ccc4Cl)cc(=O)c3c2F)C1] | (R)-4-chloro-3- (5,7-difluoro-6- (3-methoxypyrrolidin- 1-yl)-4-oxo-1,4- dihydroquinolin- 2-yl)benzonitrile | (400 MHz, METHANOL-d₄): δ 8.16 (d, J = 2.0 Hz, 1H), 8.01-8.05 (m, 1H), 7.91 (d, J = 8.6 Hz, 1H), 7.42- 7.47 (m, 1H), 6.97 (s, 1H), 4.13 (dt, J = 4.4, 2.4 Hz, 1H), 3.88-4.00 (m, 2H), 3.66 (s, 2H), 3.38- 3.40 (m, 3H), 2.02-2.19 (m, 2H). | Calculated m/z 416.1, found m/z 416.0 |
| 107 | 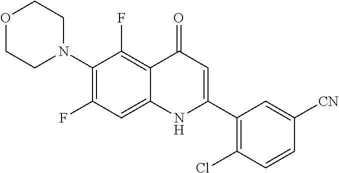 [SMILES: N#Cc1ccc(Cl)c(-c2cc(=O)c3c(F)c(N4CCOCC4)c(F)cc3[nH]2)c1] | 4-chloro-3-(5,7- difluoro-6- morpholino-4- oxo-1,4- dihydroquinolin- 2-yl)benzonitrile | (400 MHz, METHANOL-d₄) δ 8.04 (d, J = 1.8 Hz, 1H), 7.92 (dd, J = 2.0, 8.4 Hz, 1H), 7.85-7.78 (m, 1H), 7.12 (br d, J = 11.8 Hz, 1H), 6.23 (br s, 1H), 3.88-3.77 (m, 4H), 3.26- 3.19 (m, 4H) | Calculated m/z 402.1, found m/z 402.0 |
| 108 | 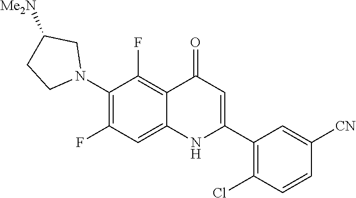 [SMILES: CN(C)[C@H]1CCN(c2c(F)cc3[nH]c(-c4cc(C#N)ccc4Cl)cc(=O)c3c2F)C1] | (S)-4-chloro-3- (6-(3- (dimethylamino) pyrrolidin-1-yl)- 5,7- difluoro-4-oxo- 1,4- dihydroquinolin- 2-yl)benzonitrile | (400 MHz, METHANOL-d₄) δ 8.03 (d, J = 1.8 Hz, 1H), 7.94-7.89 (m, 1H), 7.84- 7.80 (m, 1H), 7.11 (dd, J = 1.2, 12.8 Hz, 1H), 6.23 (s, 1H), 3.74-3.61 (m, 2H), 3.61-3.52 (m, 2H), 3.22-3.11 (m, 1H), 2.47 (s, 6H), 2.37-2.20 (m, 1H), 2.02-1.88 (m, 1H) | Calculated m/z 429.1, found m/z 429.1 |
| 109 | 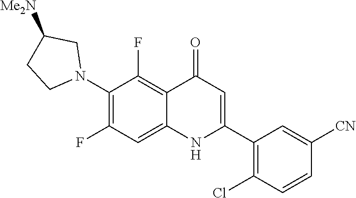 [SMILES: CN(C)[C@@H]1CCN(c2c(F)cc3[nH]c(-c4cc(C#N)ccc4Cl)cc(=O)c3c2F)C1] | (R)-4-chloro-3- (6-(3- (dimethylamino) pyrrolidin-1- yl)-5,7- difluoro-4-oxo-1,4- dihydroquinolin- 2-yl)benzonitrile | (400 MHz, METHANOL-d₄) δ 8.49 (s, 1H), 8.02 (d, J = 1.8 Hz, 1H), 7.92 (dd, J = 1.8, 8.4 Hz, 1H), 7.82 (d, J = 8.4 Hz, 1H), 7.14 (br d, J = 11.8 Hz, 1H), 6.24 (s, 1H), 3.77-3.51 (m, 5H), 2.73 (s, 6H), 2.55-2.30 (m, 1H), 2.12 (br d, J = 7.6 Hz, 1H) | Calculated m/z 429.1, found m/z 429.0 |
| 110 | 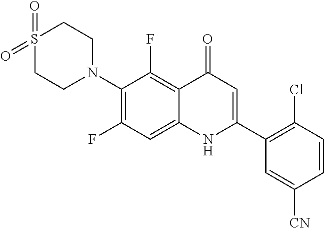 [SMILES: N#Cc1ccc(Cl)c(-c2cc(=O)c3c(F)c(N4CCS(=O)(=O)CC4)c(F)cc3[nH]2)c1] | 4-chloro-3-(6- (1,1-dioxido- thiomorpholino)- 5,7- difluoro-4-oxo-1,4- dihydroquinolin- 2-yl)benzonitrile | (400 MHz, DMSO-d₆) δ 8.22 (d, J = 1.8 Hz, 1H), 8.09 (dd, J = 1.8, 8.4 Hz, 1H), 7.92 (d, J = 8.6 Hz, 1H), 7.34 (br d, J = 11.8 Hz, 1H), 6.27 (br s, 1H), 3.57 (br s, 4H), 3.27 (br s, 4H) | Calculated m/z 450.0, found m/z 450.0 |
| 111 | 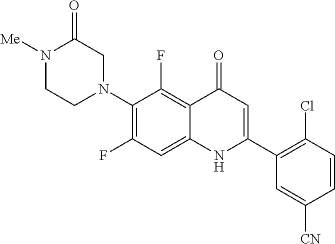 [SMILES: CN1CCN(c2c(F)cc3[nH]c(-c4cc(C#N)ccc4Cl)cc(=O)c3c2F)CC1=O] | 4-chloro-3-(5,7- difluoro-6-(4- methyl-3- oxopiperazin-1- yl)-4-oxo-1,4- dihydroquinolin- 2-yl)benzonitrile | (400 MHz, DMSO-d₆) δ ppm 12.03 (br s, 1 H) 8.21 (br s, 1 H) 8.08 (br d, J = 8.2 Hz, 1 H) 7.92 (d, J = 8.4 Hz, 1 H) 7.08-7.23 (m, 1 H) 6.03 (br s, 1 H) 3.69-3.78 (m, 2 H) 3.57 (m, 2 H) 3.37-3.49 (m, 2 H, 2.91 (s, 3 H) | Calculated m/z 429.1, found m/z 429.0 |
| 112 | 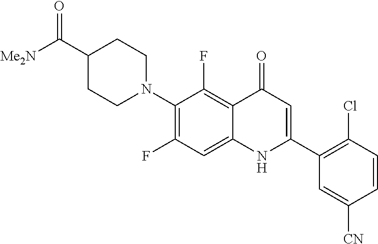 [SMILES: CN(C)C(=O)C1CCN(c2c(F)cc3[nH]c(-c4cc(C#N)ccc4Cl)cc(=O)c3c2F)CC1] | 1-(2-(2-chloro- 5- cyanophenyl)- 5,7-difluoro-4- oxo-1,4- dihydroquinolin- 6-yl)-N,N- dimethylpiperidine- 4- carboxamide | (400 MHz, DMSO-d₆) δ 12.40- 11.42 (m, 1H), 8.23- 8.16 (m, 1H), 8.06 (s, 1H), 7.91 (d, J = 8.4 Hz, 1H), 7.14 (br d, J = 11.0 Hz, 1H), 6.06 (br d, J = 9.6 Hz, 1H), 3.16 (br s, 4H), 3.06 (s, 3H), 2.83 (s, 4H), 1.69 (br d, J = 3.2 Hz, 4H) | Calculated m/z 471.1, found m/z 471.0 |
| 113 | 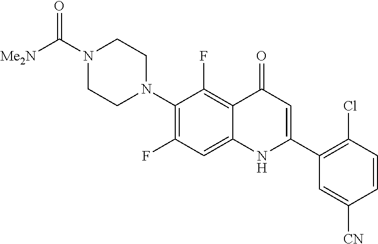 [SMILES: CN(C)C(=O)N1CCN(c2c(F)cc3[nH]c(-c4cc(C#N)ccc4Cl)cc(=O)c3c2F)CC1] | 4-(2-(2-chloro- 5- cyanophenyl)- 5,7-difluoro-4- oxo-1,4- dihydroquinolin- 6-yl)-N,N- dimethylpiperazine- 1-carboxamide | (400 MHz, DMSO-d₆) δ 12.52- 11.36 (m, 1H), 8.20 (d, J = 1.8 Hz, 1H), 8.06 (br d, J = 2.0 Hz, 1H), 7.91 (d, J = 8.4 Hz, 1H), 7.15 (br d, J = 9.0 Hz, 1H), 6.19-5.94 (m, 1H), 3.26-3.20 (m, 4H), 3.17-3.07 (m, 4H), 2.78 (s, 6H) | Calculated m/z 472.1, found m/z 472.0 |
| 114 | 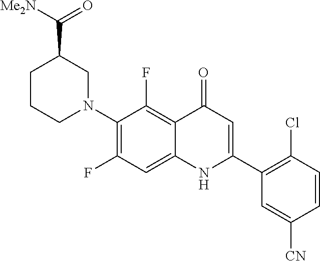 [SMILES: CN(C)C(=O)[C@@H]1CCCN(c2c(F)cc3[nH]c(-c4cc(C#N)ccc4Cl)cc(=O)c3c2F)C1] | (R)-1-(2-(2- chloro-5- cyanophenyl)- 5,7-difluoro-4- oxo-1,4- dihydroquinolin- 6-yl)-N,N- dimethylpiperidine- 3- carboxamide | (400 MHz, METHANOL-d4) δ 8.03 (d, J = 2.0 Hz, 1H), 7.93-7.89 (m, 1H), 7.81 (d, J = 8.6 Hz, 1H), 7.12-7.07 (m, 1H), 6.22 (s, 1H), 3.24 (s, 3H), 3.16 (s, 3H), 3.14-3.03 (m, 2H), 2.92 (s, 3H), 1.95 (br dd, J = 3.2, 13.0 Hz, 1H), 1.86-1.75 (m, 2H), 1.70-1.55 (m, 1H) | Calculated m/z 471.1, found m/z 471.0 |
| 115 | 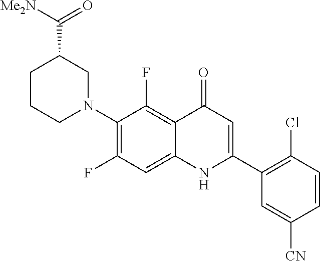 [SMILES: CN(C)C(=O)[C@H]1CCCN(c2c(F)cc3[nH]c(-c4cc(C#N)ccc4Cl)cc(=O)c3c2F)C1] | (S)-1-(2-(2- chloro-5- cyanophenyl)- 5,7-difluoro-4- oxo-1,4- dihydroquinolin- 6-yl)-N,N- dimethylpiperidine- 3-carboxamide | (400 MHz, METHANOL-d₄) δ 8.04 (d, J = 2.0 Hz, 1H), 7.96- 7.89 (m, 1H), 7.82 (d, J = 8.4 Hz, 1H), 7.11 (br d, J = 12.4 Hz, 1H), 6.24 (br s, 1H), 3.23 (br s, 3H), 3.17 (s, 3H), 3.16-3.04 (m, 2H), 2.94 (s, 3H), 2.01-1.92 (m, 1H), 1.87-1.78 (m, 2H), 1.70-1.58 (m, 1H) | Calculated m/z 471.1, found m/z 471.0 |
| 116 | 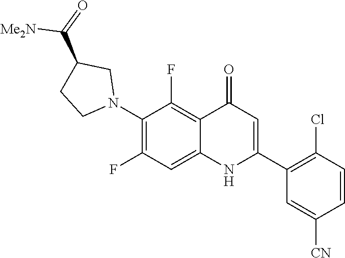 [SMILES: CN(C)C(=O)[C@@H]1CCN(c2c(F)cc3[nH]c(-c4cc(C#N)ccc4Cl)cc(=O)c3c2F)C1] | (R)-1-(2-(2- chloro-5- cyanophenyl)- 5,7-difluoro-4- oxo-1,4- dihydroquinolin- 6-yl)-N,N- dimethylpyrrolidine- 3-carboxamide | (400 MHz, DMSO-d₆) δ 11.93 (br s, 1H), 8.21 (br s, 1H), 8.06 (br d, J = 7.6 Hz, 1H), 7.90 (br d, J = 8.2 Hz, 1H), 7.09 (br d, J = 11.8 Hz, 1H), 5.95 (br s, 1H), 3.54 (br s, 3H), 3.47-3.39 (m, 2H), 3.05 (s, 3H), 2.85 (s, 3H), 2.19-1.92 (m, 2H) | Calculated m/z 457.1, found m/z 457.0 |
| 117 | 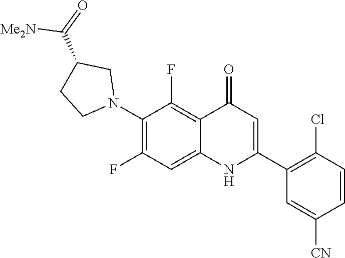 [SMILES: CN(C)C(=O)[C@H]1CCN(c2c(F)cc3[nH]c(-c4cc(C#N)ccc4Cl)cc(=O)c3c2F)C1] | (S)-1-(2-(2- chloro-5- cyanophenyl)- 5,7-difluoro-4- oxo-1,4- dihydroquinolin- 6-yl)-N,N- dimethylpyrrolidine- 3-carboxamide | (400 MHz, DMSO-d₆) δ 11.93 (br s, 1H), 8.20 (br s, 1H), 8.06 (br d, J = 7.9 Hz, 1H), 7.90 (br d, J = 8.3 Hz, 1H), 7.10 (br d, J = 4.5 Hz, 1H), 5.96 (br s, 1H), 3.55 (br s, 3H), 3.48-3.40 (m, 2H), 3.05 (s, 3H), 2.85 (s, 2H), 2.19- 1.94 (m, 2H) | Calculated m/z 457.1, found m/z 457.0 |

### Examples 118 and 119

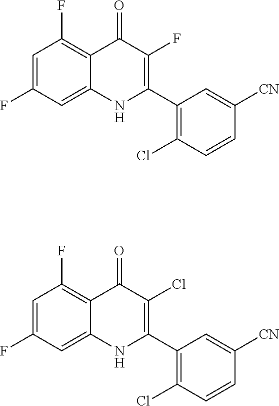 [SMILES: N#Cc1ccc(Cl)c(-c2[nH]c3cc(F)cc(F)c3c(=O)c2F)c1 ; N#Cc1ccc(Cl)c(-c2[nH]c3cc(F)cc(F)c3c(=O)c2Cl)c1]

### 4-chloro-3-(3,5,7-trifluoro-4-oxo-1,4-dihydroquinolin-2-yl)benzonitrile and 4-chloro-3-(3-chloro-5,7-difluoro-4-oxo-1,4-dihdroquinolin-2-yl)benzonitrile

4-chloro-3-(5,7-difluoro-4-oxo-1,4-dihydroquinolin-2-yl)benzonitrile (60 mg, 189 μmol, 1.0 equiv.) and Selectfluor (67.1 mg, 189 μmol, 1 equiv.) were dissolved in DMA (1.5 mL) and the solution was placed in a microwave tube. The tube was sealed and heated at 150° C. for 0.5 hour with microwave irradiation. The mixture was concentrated and the resulting residue was purified by preparative HPLC (column: Phenomenex Luna C18 75×30 mm×3 um; mobile phase: 35-50% acetonitrile in water (+0.2% formic acid)) to afford 4-chloro-3-(3,5,7-trifluoro-4-oxo-1,4-dihydroquinolin-2-yl)benzonitrile (10.4 mg, 17% yield) and 4-chloro-3-(3-chloro-5,7-difluoro-4-oxo-1,4-dihydroquinolin-2-yl)benzonitrile (2.7 mg, 3.7% yield) as a brown solids. Example 118, LCMS: calculated for [M+H] (C₁₆H₆ClF₃N₂O) requires m/z 335.0, LCMS found m/z 335.0. ¹H NMR (400 MHz, METHANOL-d₄) δ 8.14 (d, J=2.0 Hz, 1H), 7.99 (dd, J=2.0, 8.4 Hz, 1H), 7.89 (d, J=8.4 Hz, 1H), 7.10 (br d, J=9.4 Hz, 1H), 7.01 (ddd, J=2.2, 9.4, 11.8 Hz, 1H). Example 119, LCMS: calculated for [M+H](C₁₆H₆Cl₂F₂N₂O) requires m/z 351.0, LCMS found m/z 350.9. ¹H NMR (400 MHz, METHANOL-d₄) δ 8.08 (d, J=1.8 Hz, 1H), 7.98 (dd, J=2.0, 8.6 Hz, 1H), 7.88 (d, J=8.3 Hz, 1H), 7.10-6.98 (m, 2H).

### Biochemical and Cellular Assays

PPARγ-NCOR1 Recruitment Assay:

Compound potency (EC₅₀) and maximal extent of NCOR1 recruitment to PPARG were assessed a TR-FRET binding assay measuring association of a biotinylated NCOR1 ID2 peptide (Biotin-GHSFADPASNLGLEDIIRKALMG-amide) to PPARG/RXRA LBD heterodimer. Specifically, a 20 microliters of TR-FRET master mix consisting of 2 nM WT PPARG LBD (E. coli expressed, His-TEV-Q203-Y477; Uniprot ID P37231-2), 2 nM WT RXRA LBD or mutant S427F RXRA LBD (E. coli expressed, Flag-TEV-E228-T462; P19793-1), 50 nM NCOR1, 80 nM Rosiglitazone, 25 nM streptavidin-d2 (Cisbio) and 0.3 nM Anti-His Tb (Cisbio) in 25 mM MOPS pH 7.4, 25 mM KCl, 1 mM EDTA, 0.01% BSA, 0.01% Tween-20 and 1 mM TCEP was added to 384-well plates containing duplicate 10-point dose response titrations of compounds in 60 nL DMSO (0.3% f.c. DMSO (v/v)). Mixtures were incubated for 3 hours and read in an EnVision plate reader (Perkin Elmer) with Ex/Em 615/665. To determine the potency (EC₅₀) and extent of NCOR1 recruitment, TR-FRET ratios were normalized to the average ratio of DMSO control wells (0%) and to the average maximum ratio for positive control compound (T0070907 (2-chloro-5-nitro-N-4-pyridinyl-benzamide); defined as 100%) in CDD Vault and analyzed using the Levenberg-Marquardt algorithm.

PPARγ-MED1 Blockade Assay:

Compound potency (IC₅₀) and maximal extent of MED1 repulsion to PPARG were assessed a TR-FRET binding assay measuring association of a biotinylated MED1 LxxLL peptide (Biotin-VSSMAGNTKNHPMLMNLLKDNPAQ-amide) to PPARG/RXRA LBD heterodimer. Specifically, a 20 microliters of TR-FRET master mix consisting of 2 nM WT PPARG LBD (E. coli expressed, His-TEV-Q203-Y477; Uniprot ID P37231-2), 2 nM WT RXRA LBD (E. coli expressed, Flag-TEV-E228-T462; P19793-1), 350 nM NCOR1, 80 nM Rosiglitazone, 175 nM streptavidin-d₂ (Cisbio) and 0.3 nM Anti-His Tb (Cisbio) in 25 mM MOPS pH 7.4, 25 mM KCl, 1 mM EDTA, 0.01% BSA, 0.01% Tween-20 and 1 mM TCEP was added to 384-well plates containing duplicate 10-point dose response titrations of compounds in 60 nL DMSO (0.3% DMSO f.c. (v/v)). Mixtures were incubated for 3 hours and read in an EnVision plate reader (Perkin Elmer) with Ex/Em 615/665. To determine the potency (IC₅₀) and extent of MED1 repulsion, TR-FRET ratios were normalized to the average ratio of DMSO control wells (0%) and to the average minimum ratio for positive control compound (GW9662 (2-chloro-5-nitrobenzanilide); defined as 100%) in CDD Vault and analyzed using the Levenberg-Marquardt algorithm.

Bladder Cancer Pharmacodynamic Assay

5637 (PPARG amplified) and HT1197 (RXRA S427F mutation) cells were used for assessment of modulation of PPARG target genes using quantitative PCR. Cells were treated for 24 hours with PPARG inverse agonists prior to analysis of FABP4 (IDT, Cat: Hs.PT 58.20106818) and ANGPTL4 (IDT, Cat: Hs.PT 58.25480012) expression, with expression of the housekeeping gene TBP (IDT, Cat: Hs.PT 58v.39858774) used to normalize expression across samples. Quantitative PCR was performed using an ABI QuantStudio 7 Flex Reaction system. Data were analyzed and reported relative to DMSO control using the comparative Ct method (ΔΔCt).

### Table 10

For the PPARG-NCOR recruitment assay the EC₅₀ is expressed as follows, A: <10 nM, B: 10-100 nM, C: 100-1,000 nM, D: 1,000-10,000 nM, E: >10,000 nM. The % NCOR recruitment is expressed as follows, A: >100% (>the control compound, T907), B: <100% (<the control compound, T907).

For the PPARG-MED1 recruitment assay the EC₅₀ is expressed as follows, A: <10 nM, B: 10-100 nM, C: 100-1,000 nM, D: 1,000-10,000 nM, E: >10,000 nM. The % MED1 blockade is expressed as follows, A: >100% (>the control compound, GW9662), B: <100% (<the control compound, GW9662).

For the 5637 cell assay the EC₅₀ is expressed as follows, A: a10 nM, B: 10-100 nM, C: 100-1,000 nM, D: 1,000-10,000 nM, E: >10,000 nM, ND: not determined. The % inhibition of FABP4, a PPARG target gene, at 100 nM compound concentration is expressed as percentage of a DMSO control experiment.

For the HT1197 cell assay the EC₅₀ is expressed as follows, A: 10 nM, B: 10-100 nM, C: 100-1,000 nM, D: 1,000-10,000 nM, E: >10,000 nM, ND: not determined. The % inhibition of ANGPTL4, a PPARG target gene, at 100 nM compound concentration is expressed as percentage of a DMSO control experiment.

| | | PPARG-MED1 | | |
|---|---|---|---|---|
| | PPARG-NCOR | blockade assay | 5637 cell assay | HT1197 cell assay |
| | recruitment assay | EC₅₀ - | EC₅₀ - | EC₅₀ - |
| | EC₅₀ - % NCOR | % MED1 blockade | % inhibition of | % inhibition of |
| Example | recruitment | relative to | FABP4 @ | ANGPTL4 @ |
| number | relative to T907 | GW9662 | 100 nM | 100 nM |
| 1 | C-A | C-A | ND-95% | 5.0 nM-88% |
| 2 | C-A | C-A | ND-93% | ND-83% |
| 3 | D-A | D-A | ND-ND | ND-ND |
| 4 | B-A | B-A | ND-94% | ND-90% |
| 5 | D-A | D-A | ND-90% | ND-86% |
| 6 | C-A | C-A | ND-93% | ND-ND |
| 7 | C-A | C-A | ND-ND | ND-90% |
| 8 | E-B | E-B | ND-ND | ND-ND |
| 9 | D-A | D-A | ND-ND | ND-ND |
| 10 | E-A | D-A | ND-ND | ND-ND |
| 11 | B-A | C-A | ND-94% | ND-88% |
| 12 | E-B | D-B | ND-ND | ND-ND |
| 13 | D-B | D-A | ND-ND | ND-17% |
| 14 | A-B | A-A | ND-ND | ND-38% |
| 15 | C-A | B-A | ND-ND | ND-89% |
| 16 | C-A | C-A | ND-ND | ND-88% |
| 17 | D-A | D-A | ND-ND | ND-66% |
| 18 | C-A | C-A | ND-ND | ND-83% |
| 19 | D-A | D-A | ND-ND | ND-75% |
| 20 | C-A | C-A | ND-ND | ND-91% |
| 21 | D-A | D-A | ND-ND | ND-87% |
| 22 | C-A | C-A | ND-ND | ND-29% |
| 23 | C-A | C-A | ND-ND | ND-55% |
| 24 | C-B | C-B | ND-ND | ND-69% |
| 25 | D-A | D-A | ND-ND | ND-22% |
| 26 | E-B | D-B | ND-ND | ND-21% |
| 27 | D-A | D-A | ND-ND | ND-47% |
| 28 | E-B | C-B | ND-ND | ND-0% |
| 29 | D-A | D-A | ND-ND | ND-38% |
| 30 | C-A | C-A | ND-ND | ND-39% |
| 31 | C-A | C-A | ND-ND | ND-90% |
| 32 | C-A | C-A | ND-ND | ND-31% |
| 33 | C-A | C-A | ND-ND | ND-ND |
| 34 | C-A | C-A | ND-ND | ND-79% |
| 35 | C-A | C-A | ND-ND | ND-54% |
| 36 | B-A | B-A | ND-ND | ND-65% |
| 37 | C-A | C-A | ND-ND | ND-87% |
| 38 | C-A | C-A | ND-ND | ND-80% |
| 39 | C-A | C-A | ND-ND | ND-33% |
| 40 | C-A | C-A | ND-ND | ND-72% |
| 41 | B-A | C-A | ND-ND | ND-72% |
| 42 | C-A | C-A | ND-ND | ND-74% |
| 43 | C-A | C-A | ND-ND | ND-75% |
| 44 | C-A | C-A | ND-ND | ND-75% |
| 45 | C-A | C-A | ND-ND | ND-66% |
| 46 | C-A | C-A | ND-ND | ND-80% |
| 47 | C-A | C-A | ND-ND | ND-66% |
| 48 | D-A | D-A | ND-ND | ND-47% |
| 49 | C-A | C-A | ND-ND | ND-52% |
| 50 | C-A | C-A | ND-ND | ND-80% |
| 51 | C-A | C-A | ND-ND | ND-56% |
| 52 | D-A | D-A | ND-ND | ND-29% |
| 53 | C-A | C-A | ND-ND | ND-36% |
| 54 | C-A | C-A | ND-ND | ND-20% |
| 55 | C-A | C-A | ND-ND | ND-65% |
| 56 | C-A | C-A | ND-ND | ND-26% |
| 57 | C-A | C-A | ND-ND | ND-87% |
| 58 | C-A | C-A | ND-ND | ND-88% |
| 59 | B-A | B-A | ND-ND | ND-42% |
| 60 | C-A | C-A | ND-ND | ND-80% |
| 61 | C-A | C-A | ND-ND | ND-70% |
| 62 | C-A | C-A | ND-ND | ND-40% |
| 63 | C-A | C-A | ND-ND | ND-24% |
| 64 | C-A | C-A | ND-ND | ND-59% |
| 65 | C-A | C-A | ND-ND | ND-44% |
| 66 | D-A | D-A | ND-ND | ND-45% |
| 67 | B-A | B-A | ND-ND | ND-22% |
| 68 | D-B | D-B | ND-ND | ND-28% |
| 69 | C-A | C-A | ND-ND | ND-68% |
| 70 | D-A | D-A | ND-ND | ND-66% |
| 71 | D-A | D-A | ND-ND | ND-51% |
| 72 | C-A | C-A | ND-ND | ND-79% |
| 73 | B-A | B-A | ND-ND | ND-85% |
| 74 | C-A | C-A | ND-ND | ND-12% |
| 75 | D-A | D-A | ND-ND | ND-20% |
| 76 | C-A | C-A | ND-ND | ND-39% |
| 77 | D-A | D-B | ND-ND | ND-44% |
| 78 | C-A | C-A | ND-ND | ND-36% |
| 79 | C-B | C-B | ND-ND | ND-58% |
| 80 | C-A | C-A | ND-ND | ND-47% |
| 81 | D-A | E-A | ND-ND | ND-46% |
| 82 | C-A | C-A | ND-ND | ND-70% |
| 83 | C-A | C-A | ND-ND | ND-76% |
| 84 | D-A | D-B | ND-ND | ND-67% |
| 85 | D-A | D-A | ND-ND | ND-59% |
| 86 | C-A | C-A | ND-ND | ND-75% |
| 87 | C-A | C-A | ND-ND | ND-75% |
| 88 | D-A | E-A | ND-ND | ND-41% |
| 89 | C-A | C-A | ND-ND | ND-56% |
| 90 | D-A | E-A | ND-ND | ND-0% |
| 91 | C-B | C-A | ND-ND | ND-ND |
| 92 | C-A | C-A | ND-ND | ND-24% |
| 93 | D-A | D-A | ND-ND | ND-48% |
| 94 | D-A | D-A | ND-ND | ND-27% |
| 95 | C-A | C-A | ND-ND | ND-87% |
| 96 | D-A | D-A | ND-ND | ND-82% |
| 97 | C-A | C-A | ND-ND | ND-52% |
| 98 | C-A | D-A | ND-ND | ND-87% |
| 99 | E-A | E-A | ND-ND | ND-59% |
| 100 | D-A | D-A | ND-ND | ND-87% |
| 101 | D-A | D-A | ND-ND | ND-52% |
| 102 | D-A | D-B | ND-ND | ND-81% |
| 103 | D-A | D-A | ND-ND | ND-82% |
| 104 | C-A | C-A | ND-ND | ND-78% |
| 105 | D-A | D-A | ND-ND | ND-60% |
| 106 | C-A | C-A | ND-ND | ND-75% |
| 107 | C-A | C-A | ND-ND | ND-80% |
| 108 | D-A | D-A | ND-ND | ND-65% |
| 109 | D-A | D-A | ND-ND | ND-65% |
| 110 | D-A | D-A | ND-ND | ND-37% |
| 111 | D-A | D-A | ND-ND | ND-43% |
| 112 | D-B | D-A | ND-ND | ND-66% |
| 113 | D-A | D-A | ND-ND | ND-80% |
| 114 | C-A | C-A | ND-ND | ND-60% |
| 115 | C-A | D-A | ND-ND | ND-80% |
| 116 | C-A | D-A | ND-ND | ND-84% |
| 117 | D-A | D-A | ND-ND | ND-79% |
| 118 | E-B | E-B | ND-ND | ND-ND |
| 119 | E-B | E-B | ND-ND | ND-ND |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 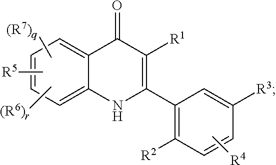
or a pharmaceutically acceptable salt thereof, wherein
R¹ is hydrogen, halo, (C₁-C₄)alkyl, or hydroxyl;
R² is halo;
R³ is cyano or nitro;
R⁴ is hydrogen, halo, (C₁-C₄)alkyl, (C₁-C₄)alkoxy, or hydroxyl;
R⁵ is halo, halo(C₁-C₄)alkyl, or cyano;
R⁶ is halo, halo(C₁-C₄)alkyl, (C₁-C₄)alkyl, or cyano;
R⁷ is halo, (C₁-C₄)alkyl, (C₁-C₄)alkoxy, halo(C₁-C₄)alkyl, halo(C₁-C₄)alkoxy, —(C₁-C₄)alkylOR^{a}, —(C₁-C₄)alkylC(O)R^{a}, —(C₁-C₄)alkylC(O)OR^{a}, —C(O)NR^{a}R^{b}, —(C₁-C₄)alkylC(O)NR^{a}R^{b}, —C(O)R^{a}, —C(O)OR^{a}, —NR^{a}R^{b}, —(C₁-C₄)alkylNR^{a}R^{b}, —C(O)NR^{a}SO₃H, —NR^{a}C(O)R^{b}, —NR^{a}C(O)OR^{b}, —NR^{a}C(S)OR^{b}, —NR^{c}C(O)N^{a}R^{b}, —NR^{a}C(S)NR^{a}R^{b}, —NR^{c}S(O)₂NR^{a}R^{b}, —C(S)R^{a}, —S(O)₂R^{a}, —S(O)R^{a}, —C(S)OR^{a}, —C(S)NR^{a}R^{b}, —NR^{a}C(S)R^{b}, —SR^{a}, phenyl, 4- to 6-membered heterocyclyl, and 5- to 7-membered heteroaryl, wherein each of said phenyl, 4- to 6-membered heterocyclyl, and 5- to 7-membered heteroaryl are optionally and independently substituted with 1 to 3 groups selected from R⁸;
R⁸ is selected from halo, (C₁-C₄)alkyl, halo(C₁-C₄)alkyl, (C₁-C₄)alkoxy, halo(C₁-C₄)alkoxy, nitro, oxo, cyano, —(C₁-C₄)alkylOR^{d}, —(C₁-C₄)alkylC(O)R^{d}, —(C₁-C₄)alkylC(O)OR^{d}, —C(O)NR^{d}R^{e}, —(C₁-C₄)alkylC(O)NR^{d}R^{e}, —C(O)R^{d}, —C(O)OR^{d}, —NR^{d}R^{e}, —(C₁-C₄)alkylNR^{d}R^{e}, —C(O)NR^{d}SO₃H, —NR^{d}C(O)R^{e}, —NR^{d}C(O)OR^{e}, —NR^{d}C(S)OR^{e}, —NR^{f}C(O)N^{d}R^{e}, —NR^{f}C(S)NR^{d}R^{e}, —NR^{f}S(O)₂NR^{d}R^{e}, —C(S)R^{d}, —S(O)₂R^{d}, —S(O)R^{d}, —C(S)OR^{d}, —C(S)NR^{d}R^{e}, —NR^{d}C(S)R^{e}, and —SR^{d};
R^{a}, R^{b}, R^{c}, R^{d}, R^{e}, and R^{f} are each independently hydrogen or (C₁-C₄)alkyl; and
q and r are each independently 0 or 1.

2. The compound of claim 1, wherein the compound is of the Formula II: 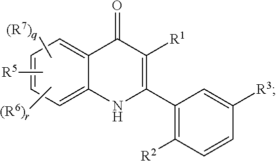
or a pharmaceutically acceptable salt thereof.

3. The compound of claim 1, or a pharmaceutically acceptable salt thereof, wherein R² is chloro.

4. The compound of claim 1, or a pharmaceutically acceptable salt thereof, wherein R³ is cyano.

5. The compound of claim 1, or a pharmaceutically acceptable salt thereof, wherein R¹ is hydrogen, fluoro, hydroxyl, or methyl.

6. The compound of claim 1, or a pharmaceutically acceptable salt thereof, wherein R¹ is hydrogen, fluoro or chloro.

7. (canceled)

8. The compound of claim 1, or a pharmaceutically acceptable salt thereof, wherein R⁵ is halo or cyano.

9. (canceled)

10. The compound of claim 1, or a pharmaceutically acceptable salt thereof, wherein R⁵ is chloro or fluoro.

11. (canceled)

12. The compound of claim 1, or a pharmaceutically acceptable salt thereof, wherein q is 1.

13. The compound of claim 1, or a pharmaceutically acceptable salt thereof, wherein r is 1.

14. The compound of claim 1, or a pharmaceutically acceptable salt thereof, wherein r is 0.

15. The compound of claim 1, or a pharmaceutically acceptable salt thereof, wherein R⁶ is halo.

16. (canceled)

17. The compound of claim 1, or a pharmaceutically acceptable salt thereof, wherein R⁷ is halo, halo(C₁-C₄)alkyl, (C₁-C₄)alkyl, (C₁-C₄)alkoxy, —(C₁-C₄)alkylOR^{a}, —C(O)NR^{a}R^{b}, phenyl, 4- to 6-membered heterocyclyl, and 5- to 7-membered heteroaryl, wherein each of said phenyl, 4- to 6-membered heterocyclyl, and 5- to 7-membered heteroaryl are optionally and independently substituted with 1 to 3 groups selected from R⁸.

18. The compound of claim 1, or a pharmaceutically acceptable salt thereof, wherein R⁷ is halo, halo(C₁-C₄)alkyl, (C₁-C₄)alkyl, (C₁-C₄)alkoxy, —(C₁-C₄)alkylOR^{a}, —C(O)NR^{a}R^{b}, phenyl, pyridinyl, pyrazolyl, and oxetanyl, wherein each of said phenyl, pyridinyl, pyrazolyl, and oxetanyl are optionally and independently substituted with 1 to 3 groups selected from R⁸.

19. The compound of claim 1, or a pharmaceutically acceptable salt thereof, wherein R⁷ is halo, halo(C₁-C₄)alkyl, (C₁-C₄)alkyl, (C₁-C₄)alkoxy, —(C₁-C₄)alkylOR^{a}, —(C₁-C₄)alkylC(O)NR^{a}R^{b}, —(C₁-C₄)alkylC(O)OR^{a}, —C(O)NR^{a}R^{b}, phenyl, 4- to 6-membered heterocyclyl, and 5- to 7-membered heteroaryl, wherein each of said phenyl, 4- to 6-membered heterocyclyl, and 5- to 7-membered heteroaryl are optionally and independently substituted with 1 to 3 groups selected from R⁸.

20. (canceled)

21. The compound of claim 1, or a pharmaceutically acceptable salt thereof, wherein R⁸ is selected from halo, (C₁-C₄)alkyl, halo(C₁-C₄)alkyl, (C₁-C₄)alkoxy, —(C₁-C₄)alkylOR^{d}, —(C₁-C₄)alkylNR^{d}R^{e}, —(C₁-C₄)alkylC(O)OR^{d}, halo(C₁-C₄)alkoxy, —C(O)OR^{d}, —(C₁-C₄)alkylC(O)NR^{d}R^{e}, —C(O)NR^{d}R^{e}, oxo, cyano, —C(O)R^{d}, —NR^{d}R^{e}, and —S(O)₂R^{d}.

22. The compound of claim 1, or a pharmaceutically acceptable salt thereof, wherein R⁸ is selected from halo, (C₁-C₄)alkyl, halo(C₁-C₄)alkyl, (C₁-C₄)alkoxy, halo(C₁-C₄)alkoxy, oxo, and cyano.

23. (canceled)

24. The compound of claim 1, wherein the compound is of the structural formula: 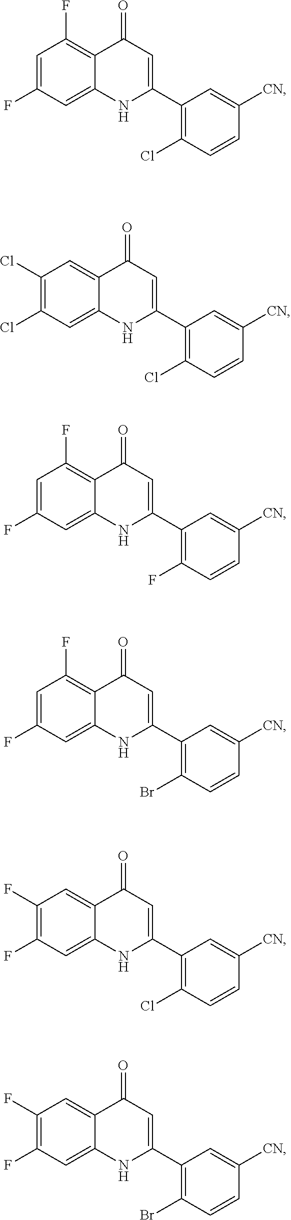 [SMILES: N#Cc1ccc(Cl)c(-c2cc(=O)c3c(F)cc(F)cc3[nH]2)c1 ; N#Cc1ccc(Cl)c(-c2cc(=O)c3cc(Cl)c(Cl)cc3[nH]2)c1 ; N#Cc1ccc(F)c(-c2cc(=O)c3c(F)cc(F)cc3[nH]2)c1 ; N#Cc1ccc(Br)c(-c2cc(=O)c3c(F)cc(F)cc3[nH]2)c1 ; N#Cc1ccc(Cl)c(-c2cc(=O)c3cc(F)c(F)cc3[nH]2)c1 ; N#Cc1ccc(Br)c(-c2cc(=O)c3cc(F)c(F)cc3[nH]2)c1] 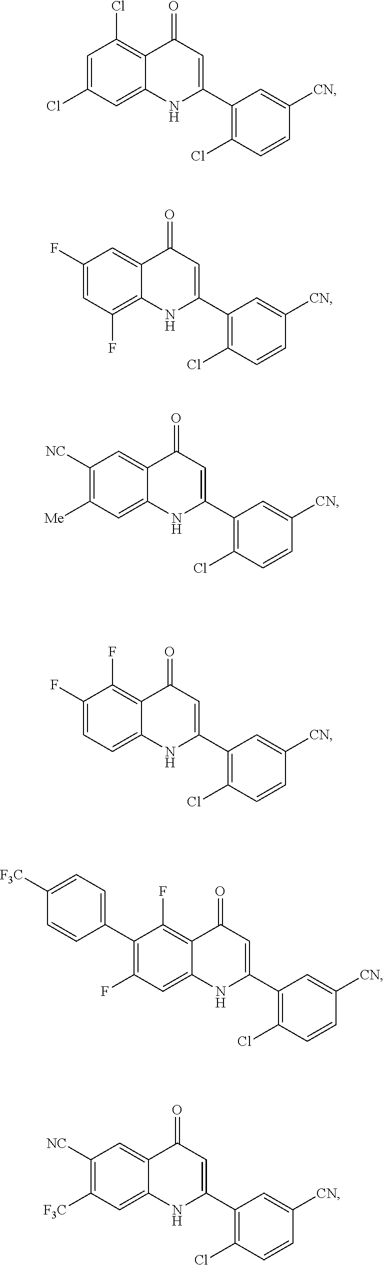 [SMILES: N#Cc1ccc(Cl)c(-c2cc(=O)c3c(Cl)cc(Cl)cc3[nH]2)c1 ; N#Cc1ccc(Cl)c(-c2cc(=O)c3cc(F)cc(F)c3[nH]2)c1 ; Cc1cc2[nH]c(-c3cc(C#N)ccc3Cl)cc(=O)c2cc1C#N ; N#Cc1ccc(Cl)c(-c2cc(=O)c3c(F)c(F)ccc3[nH]2)c1 ; N#Cc1ccc(Cl)c(-c2cc(=O)c3c(F)c(-c4ccc(C(F)(F)F)cc4)c(F)cc3[nH]2)c1 ; N#Cc1ccc(Cl)c(-c2cc(=O)c3cc(C#N)c(C(F)(F)F)cc3[nH]2)c1] 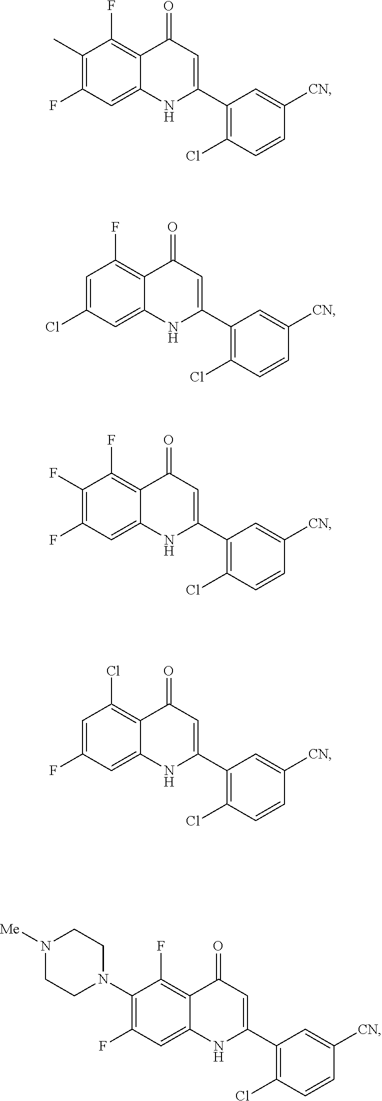 [SMILES: Cc1c(F)cc2[nH]c(-c3cc(C#N)ccc3Cl)cc(=O)c2c1F ; N#Cc1ccc(Cl)c(-c2cc(=O)c3c(F)cc(Cl)cc3[nH]2)c1 ; N#Cc1ccc(Cl)c(-c2cc(=O)c3c(F)c(F)c(F)cc3[nH]2)c1 ; N#Cc1ccc(Cl)c(-c2cc(=O)c3c(Cl)cc(F)cc3[nH]2)c1 ; CN1CCN(c2c(F)cc3[nH]c(-c4cc(C#N)ccc4Cl)cc(=O)c3c2F)CC1] 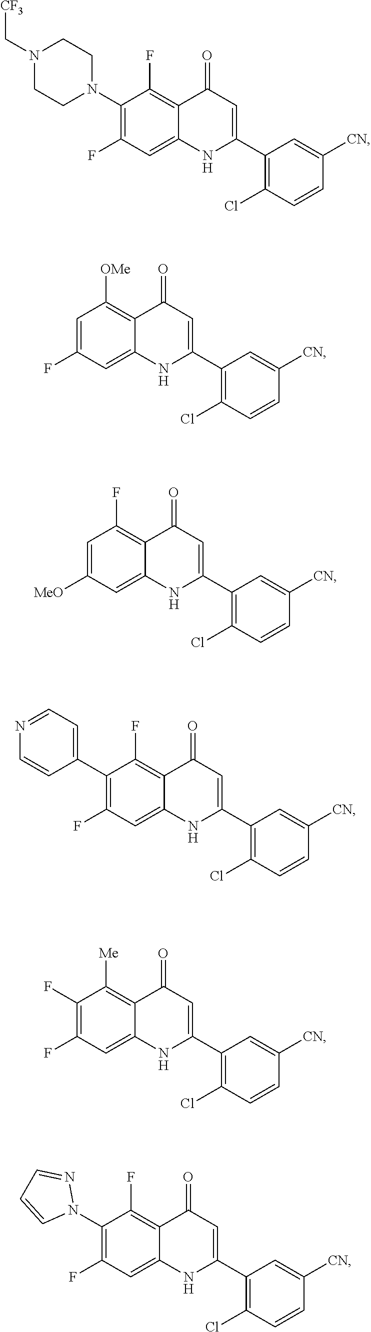 [SMILES: N#Cc1ccc(Cl)c(-c2cc(=O)c3c(F)c(N4CCN(CC(F)(F)F)CC4)c(F)cc3[nH]2)c1 ; COc1cc(F)cc2[nH]c(-c3cc(C#N)ccc3Cl)cc(=O)c12 ; COc1cc(F)c2c(=O)cc(-c3cc(C#N)ccc3Cl)[nH]c2c1 ; N#Cc1ccc(Cl)c(-c2cc(=O)c3c(F)c(-c4ccncc4)c(F)cc3[nH]2)c1 ; Cc1c(F)c(F)cc2[nH]c(-c3cc(C#N)ccc3Cl)cc(=O)c12 ; N#Cc1ccc(Cl)c(-c2cc(=O)c3c(F)c(-n4cccn4)c(F)cc3[nH]2)c1] 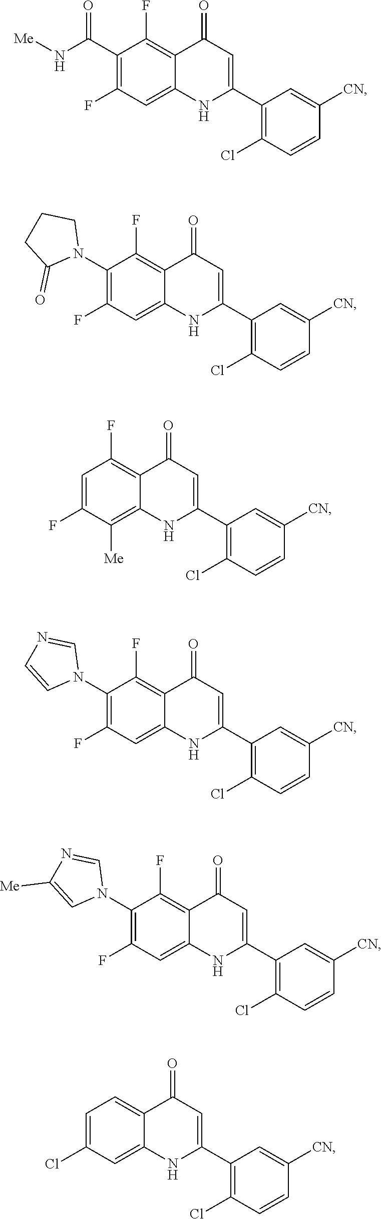 [SMILES: CNC(=O)c1c(F)cc2[nH]c(-c3cc(C#N)ccc3Cl)cc(=O)c2c1F ; N#Cc1ccc(Cl)c(-c2cc(=O)c3c(F)c(N4CCCC4=O)c(F)cc3[nH]2)c1 ; Cc1c(F)cc(F)c2c(=O)cc(-c3cc(C#N)ccc3Cl)[nH]c12 ; N#Cc1ccc(Cl)c(-c2cc(=O)c3c(F)c(-n4ccnc4)c(F)cc3[nH]2)c1 ; Cc1cn(-c2c(F)cc3[nH]c(-c4cc(C#N)ccc4Cl)cc(=O)c3c2F)cn1 ; N#Cc1ccc(Cl)c(-c2cc(=O)c3ccc(Cl)cc3[nH]2)c1] 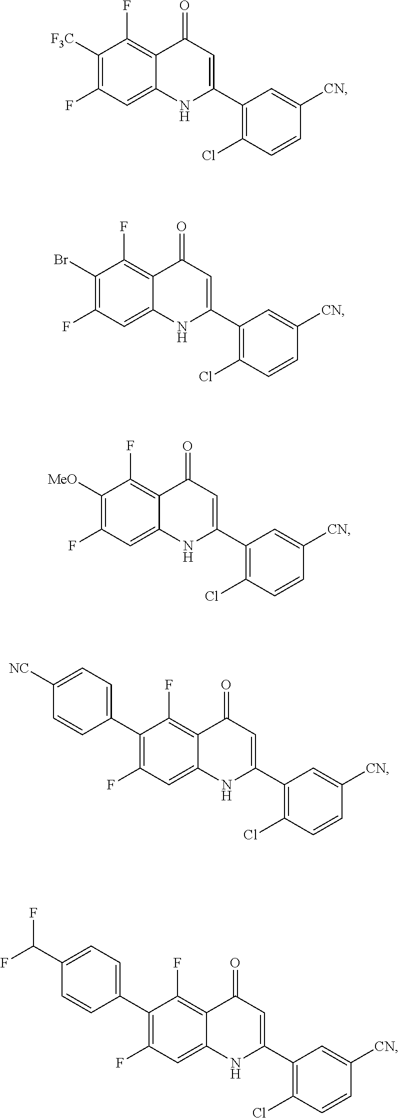 [SMILES: N#Cc1ccc(Cl)c(-c2cc(=O)c3c(F)c(C(F)(F)F)c(F)cc3[nH]2)c1 ; N#Cc1ccc(Cl)c(-c2cc(=O)c3c(F)c(Br)c(F)cc3[nH]2)c1 ; COc1c(F)cc2[nH]c(-c3cc(C#N)ccc3Cl)cc(=O)c2c1F ; N#Cc1ccc(-c2c(F)cc3[nH]c(-c4cc(C#N)ccc4Cl)cc(=O)c3c2F)cc1 ; N#Cc1ccc(Cl)c(-c2cc(=O)c3c(F)c(-c4ccc(C(F)F)cc4)c(F)cc3[nH]2)c1] 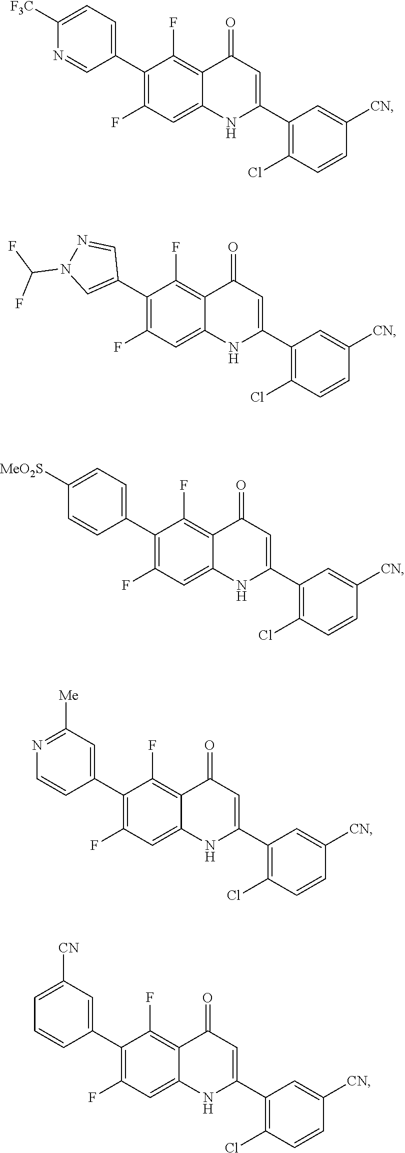 [SMILES: N#Cc1ccc(Cl)c(-c2cc(=O)c3c(F)c(-c4ccc(C(F)(F)F)nc4)c(F)cc3[nH]2)c1 ; N#Cc1ccc(Cl)c(-c2cc(=O)c3c(F)c(-c4cnn(C(F)F)c4)c(F)cc3[nH]2)c1 ; CS(=O)(=O)c1ccc(-c2c(F)cc3[nH]c(-c4cc(C#N)ccc4Cl)cc(=O)c3c2F)cc1 ; Cc1cc(-c2c(F)cc3[nH]c(-c4cc(C#N)ccc4Cl)cc(=O)c3c2F)ccn1 ; N#Cc1cccc(-c2c(F)cc3[nH]c(-c4cc(C#N)ccc4Cl)cc(=O)c3c2F)c1] 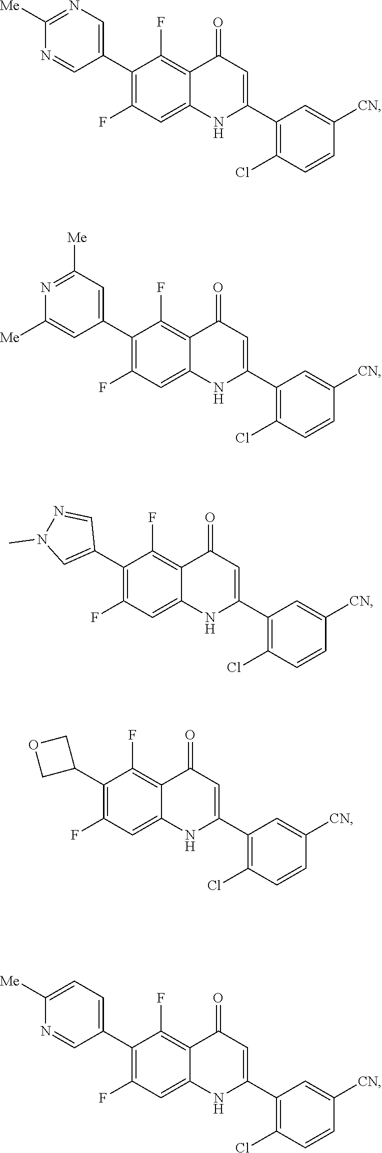 [SMILES: Cc1ncc(-c2c(F)cc3[nH]c(-c4cc(C#N)ccc4Cl)cc(=O)c3c2F)cn1 ; Cc1cc(-c2c(F)cc3[nH]c(-c4cc(C#N)ccc4Cl)cc(=O)c3c2F)cc(C)n1 ; Cn1cc(-c2c(F)cc3[nH]c(-c4cc(C#N)ccc4Cl)cc(=O)c3c2F)cn1 ; N#Cc1ccc(Cl)c(-c2cc(=O)c3c(F)c(C4COC4)c(F)cc3[nH]2)c1 ; Cc1ccc(-c2c(F)cc3[nH]c(-c4cc(C#N)ccc4Cl)cc(=O)c3c2F)cn1] 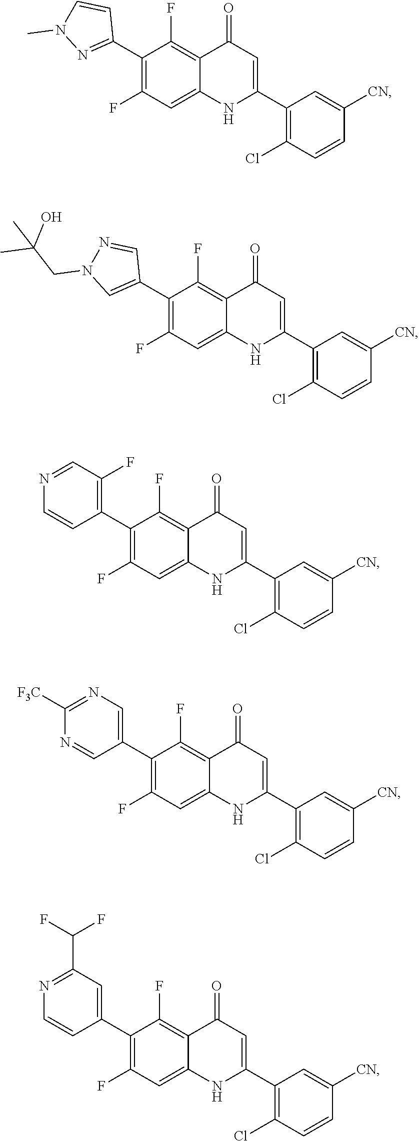 [SMILES: Cn1ccc(-c2c(F)cc3[nH]c(-c4cc(C#N)ccc4Cl)cc(=O)c3c2F)n1 ; CC(C)(O)Cn1cc(-c2c(F)cc3[nH]c(-c4cc(C#N)ccc4Cl)cc(=O)c3c2F)cn1 ; N#Cc1ccc(Cl)c(-c2cc(=O)c3c(F)c(-c4ccncc4F)c(F)cc3[nH]2)c1 ; N#Cc1ccc(Cl)c(-c2cc(=O)c3c(F)c(-c4cnc(C(F)(F)F)nc4)c(F)cc3[nH]2)c1 ; N#Cc1ccc(Cl)c(-c2cc(=O)c3c(F)c(-c4ccnc(C(F)F)c4)c(F)cc3[nH]2)c1] 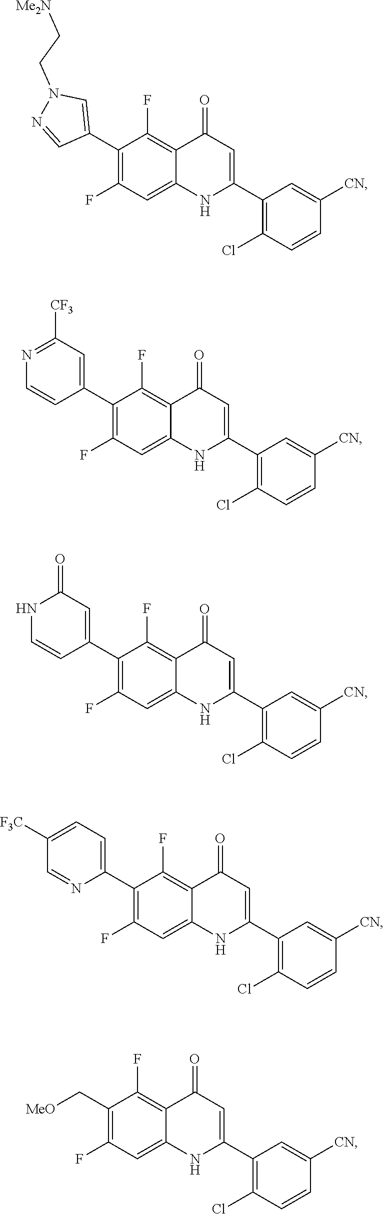 [SMILES: CN(C)CCn1cc(-c2c(F)cc3[nH]c(-c4cc(C#N)ccc4Cl)cc(=O)c3c2F)cn1 ; N#Cc1ccc(Cl)c(-c2cc(=O)c3c(F)c(-c4ccnc(C(F)(F)F)c4)c(F)cc3[nH]2)c1 ; N#Cc1ccc(Cl)c(-c2cc(=O)c3c(F)c(-c4cc[nH]c(=O)c4)c(F)cc3[nH]2)c1 ; N#Cc1ccc(Cl)c(-c2cc(=O)c3c(F)c(-c4ccc(C(F)(F)F)cn4)c(F)cc3[nH]2)c1 ; COCc1c(F)cc2[nH]c(-c3cc(C#N)ccc3Cl)cc(=O)c2c1F] 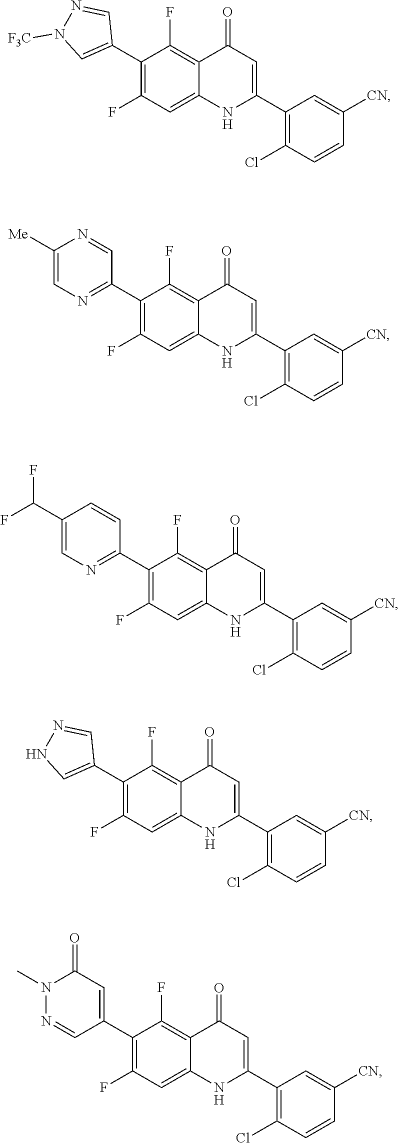 [SMILES: N#Cc1ccc(Cl)c(-c2cc(=O)c3c(F)c(-c4cnn(C(F)(F)F)c4)c(F)cc3[nH]2)c1 ; Cc1cnc(-c2c(F)cc3[nH]c(-c4cc(C#N)ccc4Cl)cc(=O)c3c2F)cn1 ; N#Cc1ccc(Cl)c(-c2cc(=O)c3c(F)c(-c4ccc(C(F)F)cn4)c(F)cc3[nH]2)c1 ; N#Cc1ccc(Cl)c(-c2cc(=O)c3c(F)c(-c4cn[nH]c4)c(F)cc3[nH]2)c1 ; Cn1ncc(-c2c(F)cc3[nH]c(-c4cc(C#N)ccc4Cl)cc(=O)c3c2F)cc1=O] 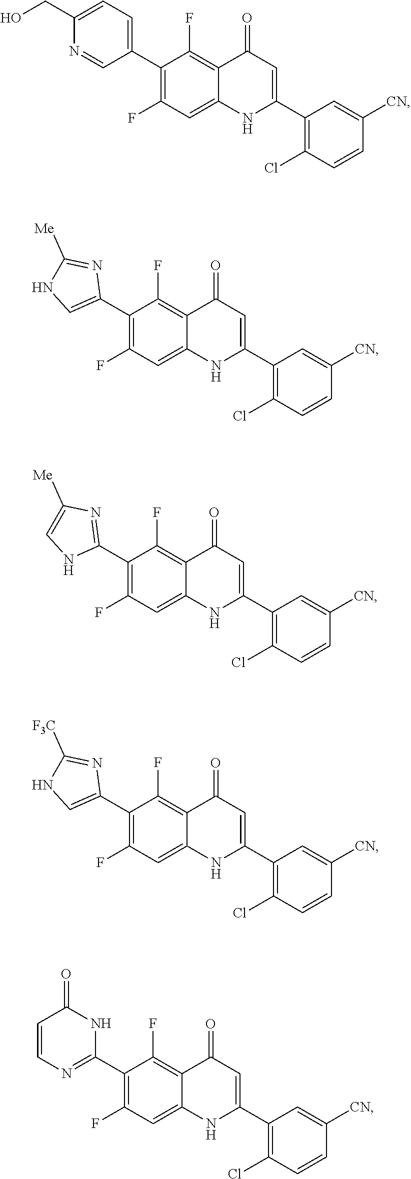 [SMILES: N#Cc1ccc(Cl)c(-c2cc(=O)c3c(F)c(-c4ccc(CO)nc4)c(F)cc3[nH]2)c1 ; Cc1nc(-c2c(F)cc3[nH]c(-c4cc(C#N)ccc4Cl)cc(=O)c3c2F)c[nH]1 ; Cc1c[nH]c(-c2c(F)cc3[nH]c(-c4cc(C#N)ccc4Cl)cc(=O)c3c2F)n1 ; N#Cc1ccc(Cl)c(-c2cc(=O)c3c(F)c(-c4c[nH]c(C(F)(F)F)n4)c(F)cc3[nH]2)c1 ; N#Cc1ccc(Cl)c(-c2cc(=O)c3c(F)c(-c4nccc(=O)[nH]4)c(F)cc3[nH]2)c1] 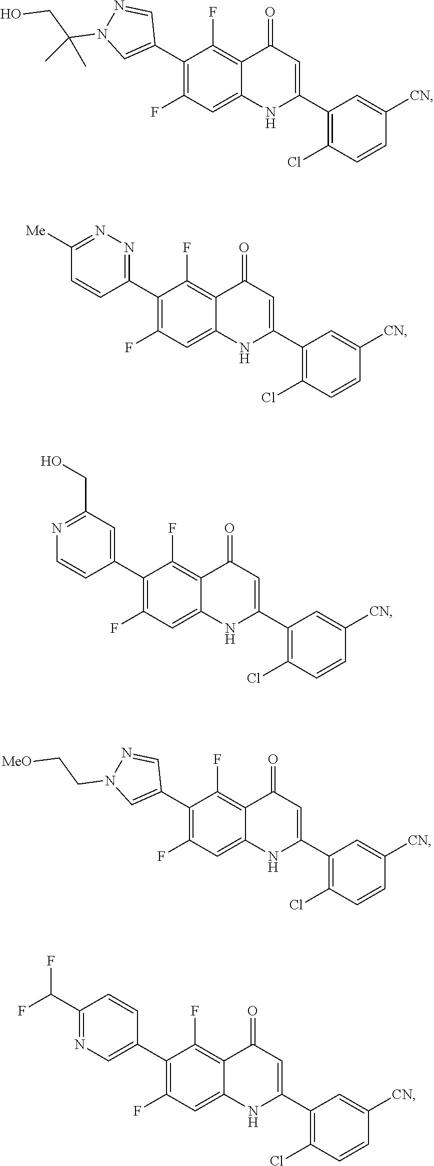 [SMILES: CC(C)(CO)n1cc(-c2c(F)cc3[nH]c(-c4cc(C#N)ccc4Cl)cc(=O)c3c2F)cn1 ; Cc1ccc(-c2c(F)cc3[nH]c(-c4cc(C#N)ccc4Cl)cc(=O)c3c2F)nn1 ; N#Cc1ccc(Cl)c(-c2cc(=O)c3c(F)c(-c4ccnc(CO)c4)c(F)cc3[nH]2)c1 ; COCCn1cc(-c2c(F)cc3[nH]c(-c4cc(C#N)ccc4Cl)cc(=O)c3c2F)cn1 ; N#Cc1ccc(Cl)c(-c2cc(=O)c3c(F)c(-c4ccc(C(F)F)nc4)c(F)cc3[nH]2)c1] 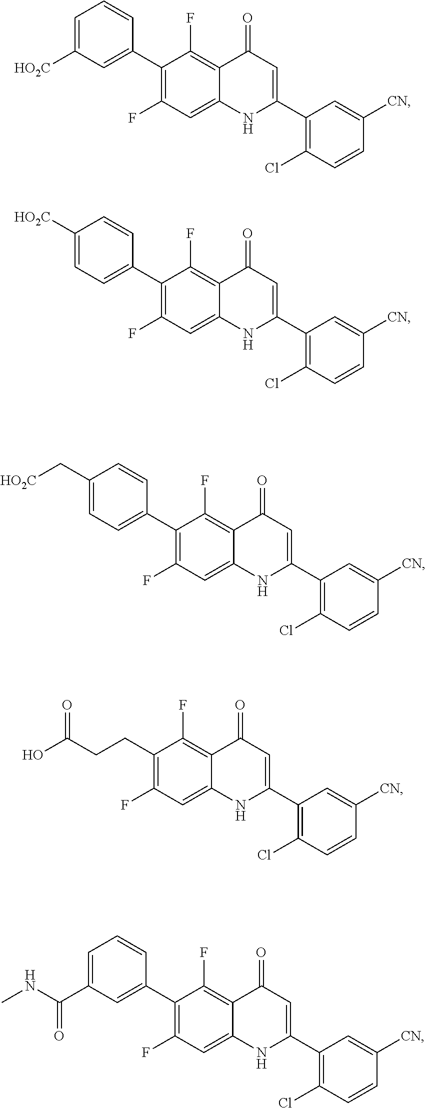 [SMILES: N#Cc1ccc(Cl)c(-c2cc(=O)c3c(F)c(-c4cccc(C(=O)O)c4)c(F)cc3[nH]2)c1 ; N#Cc1ccc(Cl)c(-c2cc(=O)c3c(F)c(-c4ccc(C(=O)O)cc4)c(F)cc3[nH]2)c1 ; N#Cc1ccc(Cl)c(-c2cc(=O)c3c(F)c(-c4ccc(CC(=O)O)cc4)c(F)cc3[nH]2)c1 ; N#Cc1ccc(Cl)c(-c2cc(=O)c3c(F)c(CCC(=O)O)c(F)cc3[nH]2)c1 ; CNC(=O)c1cccc(-c2c(F)cc3[nH]c(-c4cc(C#N)ccc4Cl)cc(=O)c3c2F)c1] 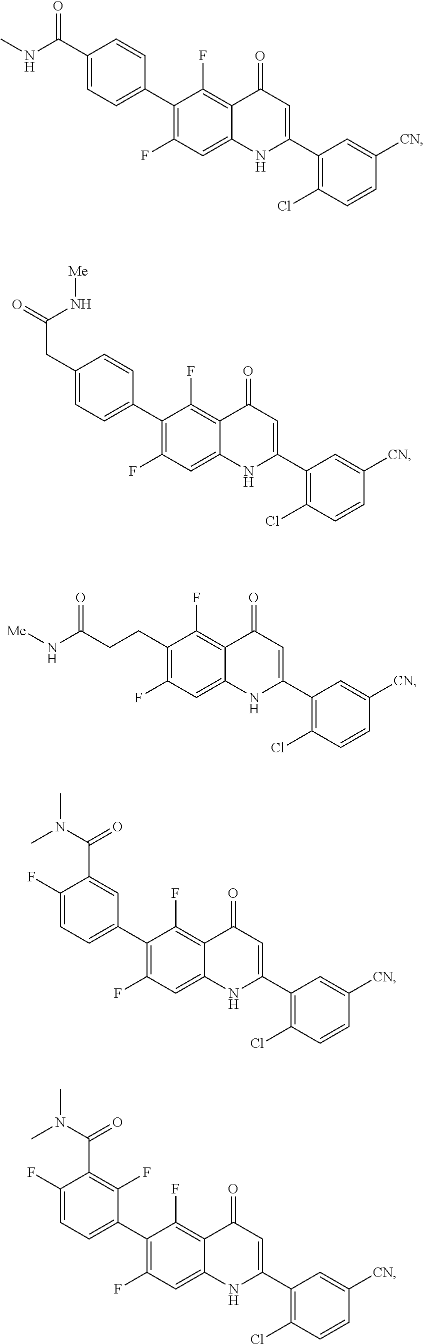 [SMILES: CNC(=O)c1ccc(-c2c(F)cc3[nH]c(-c4cc(C#N)ccc4Cl)cc(=O)c3c2F)cc1 ; CNC(=O)Cc1ccc(-c2c(F)cc3[nH]c(-c4cc(C#N)ccc4Cl)cc(=O)c3c2F)cc1 ; CNC(=O)CCc1c(F)cc2[nH]c(-c3cc(C#N)ccc3Cl)cc(=O)c2c1F ; CN(C)C(=O)c1cc(-c2c(F)cc3[nH]c(-c4cc(C#N)ccc4Cl)cc(=O)c3c2F)ccc1F ; CN(C)C(=O)c1c(F)ccc(-c2c(F)cc3[nH]c(-c4cc(C#N)ccc4Cl)cc(=O)c3c2F)c1F] 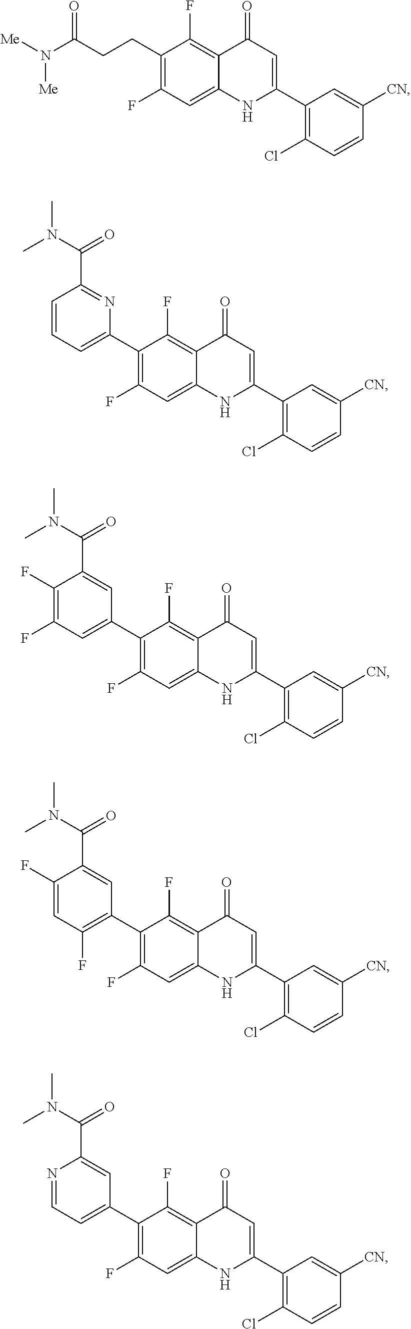 [SMILES: CN(C)C(=O)CCc1c(F)cc2[nH]c(-c3cc(C#N)ccc3Cl)cc(=O)c2c1F ; CN(C)C(=O)c1cccc(-c2c(F)cc3[nH]c(-c4cc(C#N)ccc4Cl)cc(=O)c3c2F)n1 ; CN(C)C(=O)c1cc(-c2c(F)cc3[nH]c(-c4cc(C#N)ccc4Cl)cc(=O)c3c2F)cc(F)c1F ; CN(C)C(=O)c1cc(-c2c(F)cc3[nH]c(-c4cc(C#N)ccc4Cl)cc(=O)c3c2F)c(F)cc1F ; CN(C)C(=O)c1cc(-c2c(F)cc3[nH]c(-c4cc(C#N)ccc4Cl)cc(=O)c3c2F)ccn1] 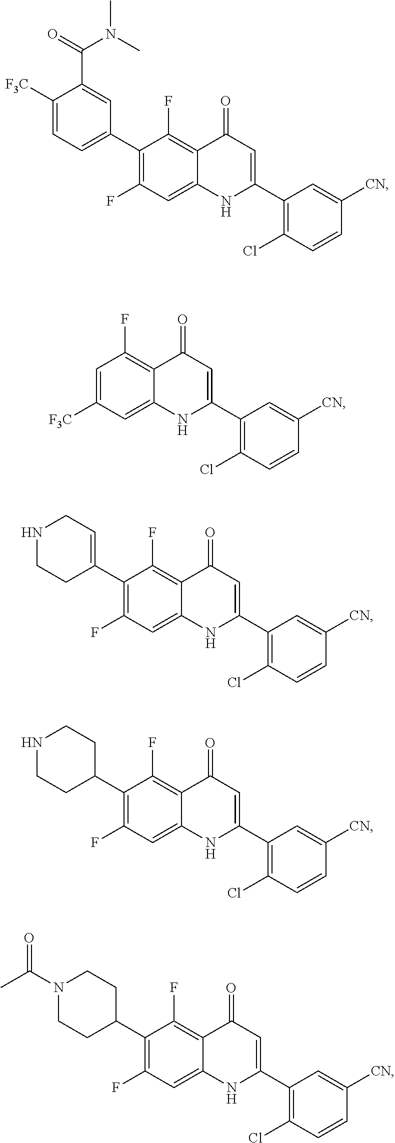 [SMILES: CN(C)C(=O)c1cc(-c2c(F)cc3[nH]c(-c4cc(C#N)ccc4Cl)cc(=O)c3c2F)ccc1C(F)(F)F ; N#Cc1ccc(Cl)c(-c2cc(=O)c3c(F)cc(C(F)(F)F)cc3[nH]2)c1 ; N#Cc1ccc(Cl)c(-c2cc(=O)c3c(F)c(C4=CCNCC4)c(F)cc3[nH]2)c1 ; N#Cc1ccc(Cl)c(-c2cc(=O)c3c(F)c(C4CCNCC4)c(F)cc3[nH]2)c1 ; CC(=O)N1CCC(c2c(F)cc3[nH]c(-c4cc(C#N)ccc4Cl)cc(=O)c3c2F)CC1] 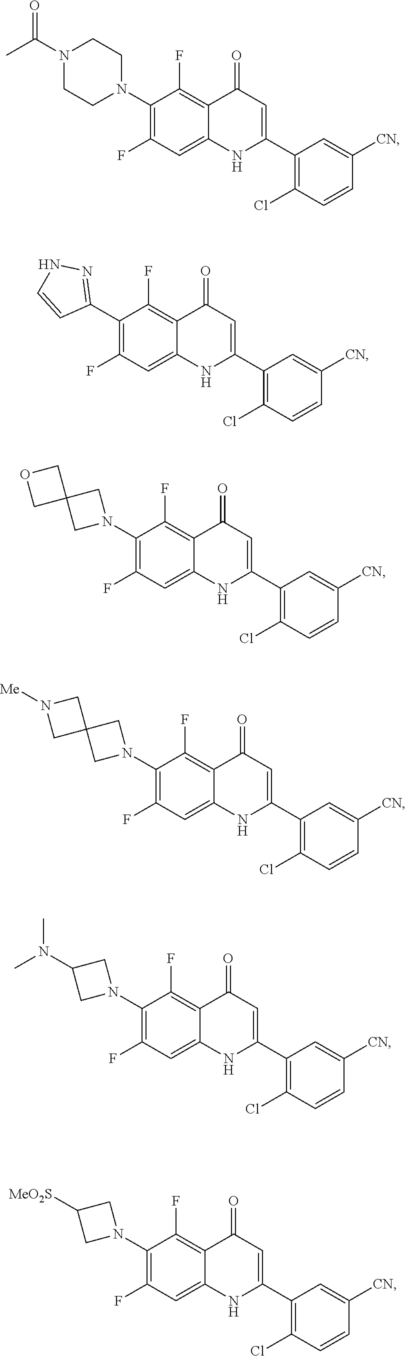 [SMILES: CC(=O)N1CCN(c2c(F)cc3[nH]c(-c4cc(C#N)ccc4Cl)cc(=O)c3c2F)CC1 ; N#Cc1ccc(Cl)c(-c2cc(=O)c3c(F)c(-c4cc[nH]n4)c(F)cc3[nH]2)c1 ; N#Cc1ccc(Cl)c(-c2cc(=O)c3c(F)c(N4CC5(COC5)C4)c(F)cc3[nH]2)c1 ; CN1CC2(C1)CN(c1c(F)cc3[nH]c(-c4cc(C#N)ccc4Cl)cc(=O)c3c1F)C2 ; CN(C)C1CN(c2c(F)cc3[nH]c(-c4cc(C#N)ccc4Cl)cc(=O)c3c2F)C1 ; CS(=O)(=O)C1CN(c2c(F)cc3[nH]c(-c4cc(C#N)ccc4Cl)cc(=O)c3c2F)C1] 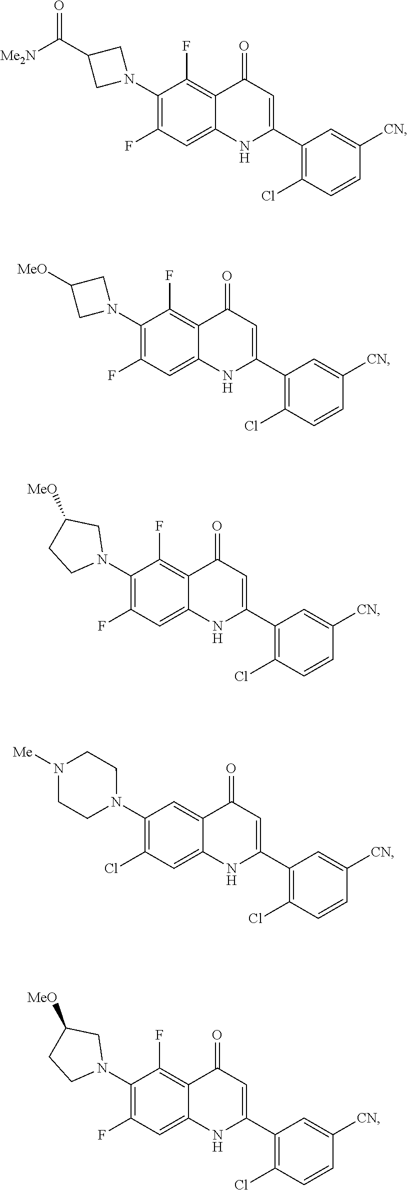 [SMILES: CN(C)C(=O)C1CN(c2c(F)cc3[nH]c(-c4cc(C#N)ccc4Cl)cc(=O)c3c2F)C1 ; COC1CN(c2c(F)cc3[nH]c(-c4cc(C#N)ccc4Cl)cc(=O)c3c2F)C1 ; CO[C@H]1CCN(c2c(F)cc3[nH]c(-c4cc(C#N)ccc4Cl)cc(=O)c3c2F)C1 ; CN1CCN(c2cc3c(=O)cc(-c4cc(C#N)ccc4Cl)[nH]c3cc2Cl)CC1 ; CO[C@@H]1CCN(c2c(F)cc3[nH]c(-c4cc(C#N)ccc4Cl)cc(=O)c3c2F)C1] 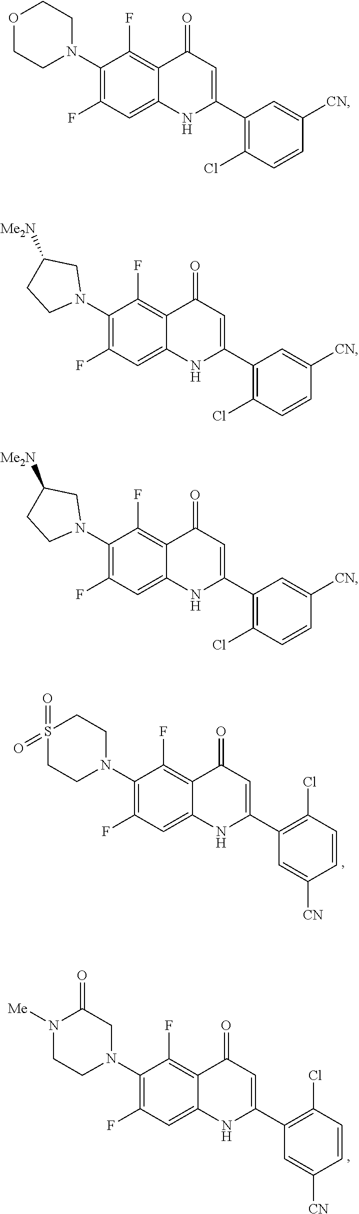 [SMILES: N#Cc1ccc(Cl)c(-c2cc(=O)c3c(F)c(N4CCOCC4)c(F)cc3[nH]2)c1 ; CN(C)[C@H]1CCN(c2c(F)cc3[nH]c(-c4cc(C#N)ccc4Cl)cc(=O)c3c2F)C1 ; CN(C)[C@@H]1CCN(c2c(F)cc3[nH]c(-c4cc(C#N)ccc4Cl)cc(=O)c3c2F)C1 ; N#Cc1ccc(Cl)c(-c2cc(=O)c3c(F)c(N4CCS(=O)(=O)CC4)c(F)cc3[nH]2)c1 ; CN1CCN(c2c(F)cc3[nH]c(-c4cc(C#N)ccc4Cl)cc(=O)c3c2F)CC1=O] 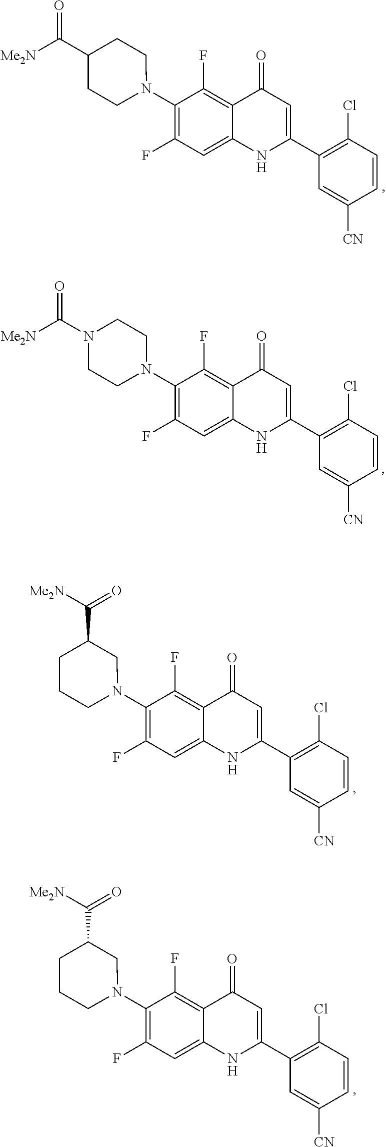 [SMILES: CN(C)C(=O)C1CCN(c2c(F)cc3[nH]c(-c4cc(C#N)ccc4Cl)cc(=O)c3c2F)CC1 ; CN(C)C(=O)N1CCN(c2c(F)cc3[nH]c(-c4cc(C#N)ccc4Cl)cc(=O)c3c2F)CC1 ; CN(C)C(=O)[C@@H]1CCCN(c2c(F)cc3[nH]c(-c4cc(C#N)ccc4Cl)cc(=O)c3c2F)C1 ; CN(C)C(=O)[C@H]1CCCN(c2c(F)cc3[nH]c(-c4cc(C#N)ccc4Cl)cc(=O)c3c2F)C1] 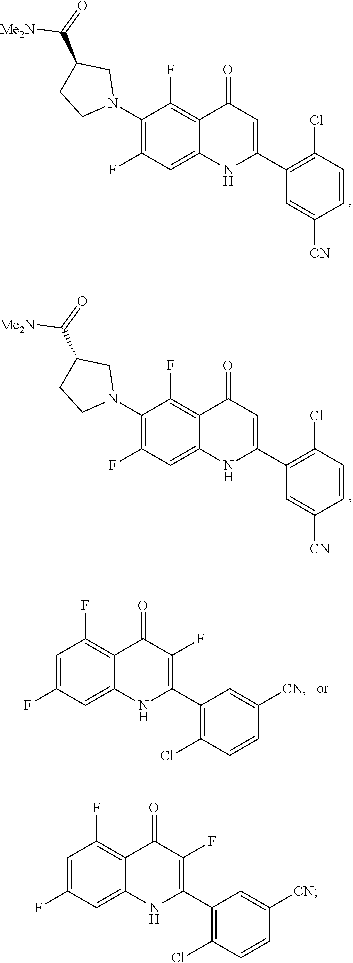 [SMILES: CN(C)C(=O)[C@@H]1CCN(c2c(F)cc3[nH]c(-c4cc(C#N)ccc4Cl)cc(=O)c3c2F)C1 ; CN(C)C(=O)[C@H]1CCN(c2c(F)cc3[nH]c(-c4cc(C#N)ccc4Cl)cc(=O)c3c2F)C1 ; N#Cc1ccc(Cl)c(-c2[nH]c3cc(F)cc(F)c3c(=O)c2F)c1 ; N#Cc1ccc(Cl)c(-c2[nH]c3cc(F)cc(F)c3c(=O)c2F)c1]
or a pharmaceutically acceptable salt of any of the foregoing.

25. A pharmaceutical composition comprising a compound of claim 1, or a pharmaceutically acceptable salt thereof; and a pharmaceutically acceptable carrier.

26. A method of treating a cancer responsive to the suppression of PPARG in a subject, comprising administering to the subject a therapeutically effective amount of a compound of claim 1, or a pharmaceutically acceptable salt thereof.

27. (canceled)

28. (canceled)
